(19) United States
(12) Patent Application Publication
(10) Pub. No.: US 2026/0085058 A1
(43) Pub. Date: Mar. 26, 2026

(54) DEGRADERS OF SON OF SEVENLESS HOMOLOG 1

(71) Applicants: Yu YUAN, Oviedo, FL (US); H. LEE MOFFITT CANCER CENTER AND RESEARCH INSTITUTE, INC., Tampa, FL (US)

(72) Inventors: Yu YUAN, Oviedo, FL (US); Hao XIE, Rochester, MN (US)

(21) Appl. No.: 19/103,367

(22) PCT Filed: Aug. 11, 2023
(86) PCT No.: PCT/US2023/030058

§ 371 Date: Feb. 12, 2025

Related U.S. Application Data
(60) Provisional application No. 63/397,596, filed on Aug. 12, 2022.

(51) Int. Cl. C07D 401/14; A61K 31/517; A61P 35/00; C07D 417/14

(52) U.S. Cl. CPC C07D 401/14; A61K 31/517; A61P 35/00; C07D 417/14

(57) ABSTRACT

This disclosure relates to compounds which are degraders of son of sevenless homolog 1 (SOS1) and their use in treating cancers, in particular KRAS-mutant cancers.

### CROSS-REFERENCE TO RELATED APPLICATIONS

This application claims the benefit of priority to U.S. Provisional Application No. 63/397,596 filed Aug. 12, 2022, the disclosure of which is incorporated herein by reference in its entirety.

### TECHNICAL FIELD

This disclosure relates to compounds useful in treating medical disorders, and more particularly to degraders of son of sevenless homolog 1 (SOS1) which are useful in treating cancers.

### BACKGROUND

Colorectal cancer (CRC) remains the third most fatal cancer in the United States, despite extensive screening efforts and advances in therapies, especially for KRAS-mutant CRC.¹ Besides chemotherapy, efficacy of targeted and immunotherapy are often limited to rare molecular subtypes of CRC, especially KRAS-mutant CRC. Currently available therapeutic strategies targeting mutant KRAS in CRC have limited clinical activity.

Approximately 41% of the patients with advanced CRC carry KRAS driver mutations, which have been associated with poor prognosis and serve as a negative predictive marker for epidermal growth factor receptor (EGFR) blockade.² Intensive efforts to directly target KRAS have focused on developing allele-specific or pan-KRAS inhibitors.³ However, mutant KRAS inhibitor alone had only modest clinical activity in CRC.⁴ Thus, it is critical to develop novel agents targeting these drivers in KRAS-mutant CRC.

SOS1, as a guanine nucleotide exchange factor (GEF), not only promotes conversion of inactive GDP-bound KRAS to active GTP-bound KRAS at the catalytic site, but also enhances its own GEF function by binding GTP-bound KRAS at an allosteric site.⁵ Targeting SOS1 with inhibitors has potential advantages over the past and current strategies for targeting mutant KRAS. SOS1 inhibitors in preclinical studies showed at least an additive effect to enhance the activity of KRAS^{G12C} inhibitors in KRAS^{G12C} cancer in vitro and in vivo models.⁶ Given its direct interaction with mutant KRAS, targeting SOS1 also has advantages over approaches to indirectly inhibit KRAS by targeting downstream RAS effector pathways, namely MAPK and PI3K. The latter strategies were largely unsuccessful due to feedback activation or incomplete pathway suppression, which was consistent with clinical observation on insufficient activity in most cancer types. Yet, dual inhibitions of MAPK and PI3K pathways suffered from on-target and off-target toxicities in the clinic.⁷ In contrast, targeting SOS1 in KRAS-mutant cancers may be more effective as it is located upstream of mutant KRAS and less toxic due to its exclusive function as a GEF. Depletion of SOS1 decreases the survival of RAS-mutant CRC and other cancer cells.⁸⁻¹⁰ Both small molecule inhibitors and agonists have been developed to modulate SOS1 function.¹¹⁻¹⁸ Recently reported SOS1 inhibitors such as BAY293 and BI3406 were able to disrupt SOS1:KRAS interaction and demonstrated antiproliferative activity towards various cancer cell lines.^{15,17} However, SOS1 inhibitors as a single agent were less effective in inhibiting RAS-driven CRC growth in cell lines, which required combination with a MEK inhibitor for enhanced in vivo activity in CRC.¹⁷ Interestingly, small structural modifications of the substituents on the quinazoline core of BI3406 switched its function as an inhibitor to an agonist.¹⁹ In addition, a series of SOS1 agonists targeting the same binding site with SOS1 inhibitors were designed and synthesized using a fragment-based drug discovery strategy.^{11-14,16,18} However, their binding affinity to SOS1 was much lower than existing SOS1 inhibitors. Some of these SOS1 agonists carried biphasic and concentration-dependent effects on KRAS signaling without further characterizations in disease-relevant cancer models.¹²⁻¹⁴

Targeted protein degradation of SOS1 as a therapeutic strategy offers several advantages over existing small molecule inhibitors of SOS1 in KRAS-mutant CRC. Targeted protein degradation with small molecules (i.e. degraders) adopts an event-driven pharmacology reducing oncoprotein function by dropping its cellular abundance.²⁰ The ability of degraders to rapidly and reversibly “knockdown” an oncoprotein of interest is complementary to genetic strategies. Compared to small molecule inhibitors, degraders often possess improved selectivity profile and efficacy.²¹ Preclinical work suggested that because degraders remove the entire targeted protein, their effects at lower concentrations are expected to be more profound and durable with less off-target toxicity. In addition to abrogating enzymatic activity of targeted protein, degraders may also remove its scaffolding function which contributes to the added efficacy.²² Hence, in the setting of insufficient single-agent activity of SOS1 inhibitor,^{17,23} the efforts to synthesize targeted SOS1 degraders and thorough evaluation of their cellular effects in patient-derived tumor models are crucial to significantly improve current therapeutic strategy in KRAS-mutant CRC.

### SUMMARY

The present disclosure provides compounds capable of facilitating degradation of son of sevenless homolog 1 (SOS1). These compounds are useful in the treatment of medical disorders such as cancers, such as KRAS-mutant cancers including KRAS-mutant colorectal cancers.

Thus, in one aspect, a compound is provided of Formula I or Formula II

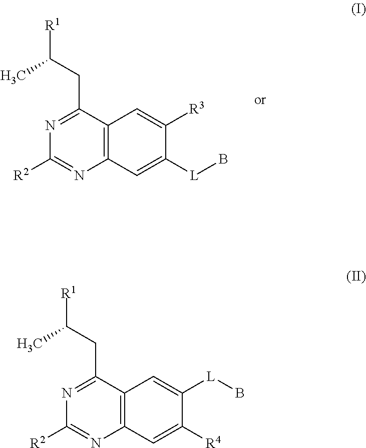

or a pharmaceutically acceptable salt or derivative thereof,
wherein all variables are as defined further herein.

In another aspect, a pharmaceutical composition is provided comprising a compound of Formula I or Formula II, or a pharmaceutically acceptable salt or derivative thereof, and a pharmaceutically acceptable carrier or excipient.

In another aspect, a method is provided for treating cancer in a subject in need thereof, the method comprising administering a therapeutically effective amount of a compound of Formula I or Formula II, or a pharmaceutically acceptable salt, derivative, or composition thereof. In some aspects, the cancer is a KRAS-mutant cancer. In some aspect s, the cancer is KRAS-mutant colorectal cancer.

The details of one or more aspects of the disclosure are set forth in the accompanying drawings and the description below. Other features, objects, and advantages of the disclosure will be apparent from the description, the drawings, and the claims.

### DESCRIPTION OF DRAWINGS

FIGS. 1A-1F demonstrate crystal structure informed design of SOS1 PROTAC degraders. (FIG. 1A) A general scheme for the design of SOS1 degraders based on the PROTAC concept utilizing SOS1 binders and common E3 ligase binders. Atoms and groups in red are essential to retain the activity of SOS1 inhibition in BAY293. Groups circled in green are putative solvent exposure sites when the corresponding molecules are in complex with SOS1. (FIG. 1B) Crystal structure of SOS1 (PDB: 6SFR) in a complex with SOS1 binder BI68BS. (FIG. 1C) Structural interaction fingerprint analysis of BI68BS in a complex with SOS1 by Schrödinger—2020 March. (FIG. 1D) The distribution of the distance between /A LBK 1103 013 (BI68BS 6-methoxy group) and /C LVY 502 N17 (lenalidomide amino group) and the distance between /A LBK 1103 014 (BI68BS 7-methoxy group) and /C LVY 502 N17 in models from protein-protein docking of cereblon E3 ligase (PDB: 4TZ4) to SOS1 (PDB: 6SFR). (FIG. 1E) The relationship between interface score (Rosetta energy units) and the distance between /A LBK 1103 013 and /C LVY 502 N17 and the distance between /A LBK 1103 014 and /C LVY 502 N17 in models from the protein-protein docking. (FIG. 1F) An example of the most favorable conformations from the protein-protein docking. In this case, distance between /A LBK 1103 013 and /C LVY 502 N17: 4.0 Å. Distance between /A LBK 1103 014 and /C LVY 502 N17: 4.7 Å.

FIG. 2 provides chemical structures of representative synthesized SOS1 PROTAC degraders.

FIGS. 3A-3B show screening of SOS1 PROTAC candidates for SOS1 degradation in SW620 after treatment for 6 hours. (FIG. 3A) SW620 cells were treated with the corresponding compounds for 6 hours followed by immunoblotting. The quantities of SOS1 treated by the compounds were normalized to that treated by DMSO as control with 1.0 as the absence of SOS1 degradation. (FIG. 3B) A summary of SOS1 degradation after treatment with the corresponding compounds for 6 hours in SW620 cells. The quantities of SOS1 were summarized as mean and standard deviation.

FIGS. 4A-4E show SOS1 degradation in colorectal cancer cell lines. (FIG. 4A) Immunoblots in SW620 cells treated with P7 for 6 hours in comparison to SOS1 inhibitor BI3406 and siSOS1. (FIG. 4B) Immunoblots in SW620 cells treated with SOS1 degrader P7 and inactive compound P2, with GAPDH used as the loading control at different time points; Cells were treated with 1 μM of P7 and P2 for indicated times, respectively. (FIG. 4C) SW620 cells were pre-treated with cycloheximide (CYC) for 1 hour at 100 μg/mL, followed by addition of DMSO or 1 μM P7. At the indicated times, cells were lysed and SOS1 levels were analyzed by western blot. (FIG. 4D) Mechanistic investigation of SOS1 degradation induced by P7 in SW620 cells. Cells were pretreated with BI3406 10 μM, lenalidomide 10 μM, MLN4924 0.5 μM, and MG132 3 M for 1 hour, followed by a 24 h treatment with P7 at 1 μM. (FIG. 4E) SOS1 degradation in colorectal cancer cells treated with SOS1 degrader P7 at different concentrations for 24 hours.

FIG. 5 shows specificity of P7 for SOS1 degradation. Volcano plot depicts the change in relative protein abundance in P7 (1 μM, 24 h)-treated SW620 cells compared with DMSO-treated cells. Protein abundance measurements were made using tandem mass spectrometry and the changes were assessed by Welch t test. The log₂ (fold change) was represented on the x-axis. The negative log₁₀ p value was represented on the y-axis. Three independent biological replicates were performed for each treatment.

FIGS. 6A-6F show Effect of SOS1 degraders in colorectal cell lines and patient-derived organoid models. (FIG. 6A) BI3406-resistant HCT116 cells were treated with either BI3406 or siSOS1 after lipofectamine for 48 hours. SOS1, pERK, ERK, c-PARP were assessed with immunoblot and quantified from three biological replicates. ns: not significant; ****: p<0.0001; * p<0.05 (FIG. 6B) SOS1 expression by IHC in colorectal cancer patient-derived organoid models. a. Positive control for SOS1 expression at 40×; b. Negative control for SOS1 expression at 40×; c. SOS1 expression of MCC19990-002 PDO after treatment with 0.2% DMSO for 24 hours at 40×; d. SOS1 expression of MCC19990-002 PDO after treatment with 1 μM P7 for 24 hours at 40×; e. SOS1 expression of MCC19990-010 PDO after treatment with 0.2% DMSO for 24 hours at 40×; f. SOS1 expression of MCC19990-010 PDO after treatment with 1 μM P7 for 24 hours at 40×; g. SOS1 expression of MCC19990-013 PDO after treatment with 0.2% DMSO for 24 hours at 40×; h. SOS1 expression of MCC19990-013 PDO after treatment with 1p M P7 for 24 hours at 40×. (FIG. 6C) H&E stain to assess morphology of colorectal cancer patient-derived organoid models. a. MCC19990-010 PDO after treatment with 0.2% DMSO for 24 hours at 40×; b. MCC19990-010 PDO after treatment with 1 μM P7 for 24 hours at 40×; c. MCC19990-013 PDO after treatment with 0.2% DMSO for 24 hours at 40×; d. MCC19990-013 PDO after treatment with 1 μM P7 for 24 hours at 40×. (FIG. 6D) Annexin V expression by IHC in colorectal cancer patient-derived organoid models. a. Negative control for Annexin V expression at 40×; b. Positive control (treated with FOLFIRI) for Annexin V expression at 40×; c. Annexin V expression of MCC19990-010 PDO after treatment with 0.2% DMSO for 24 hours at 40×; d. Annexin V expression of MCC19990-010 PDO after treatment with 1 μM P7 for 24 hours at 40×; e. Annexin V expression of MCC19990-013 PDO after treatment with 0.2% DMSO for 24 hours at 40×; f. Annexin V expression of MCC19990-013 PDO after treatment with 1 μM P7 for 24 hours at 40×. (FIG. 6E) Viability of patient-derived CRC organoid models after treatment with P7 compared to BI3406 at various concentrations for 72 hours. MCC19990-002 was resistant to both compounds. For MCC19990-006, P7 has IC₅₀ 1.4 μM; BI3406 has IC₅₀ 8.5 μM. For MCC19990-010, P7 has IC₅₀ 0.48 μM; BI3406 has IC₅₀ 1.9 μM. For MCC19990-013, P7 has IC₅₀ 1.16 μM; BI3406 has IC₅₀ 6.7 μM. (FIG. 6F) SOS1 and SOS2 mRNA expression levels after treating SW620 cells with 1 μM BI3406 and 1 μM P7, respectively, at 0, 6, 24, 48 hours.

Like reference symbols in the various drawings indicate like elements.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than ‘y’”, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than ‘y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose.

The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound or composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a cancer. The effect can be therapeutic in terms of a partial or complete cure of a disease, condition, symptom or adverse effect attributed to the disease, disorder, or condition. The term “treatment” as used herein can include any treatment of a disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

### Chemical Definitions

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

The terms for various functional groups as used herein are not intended to be limited to monovalent radicals and may include polyvalent radical groups as appropriate, such as divalent, trivalent, tetravalent, pentavalent, and hexavalent groups, and the like, based on the position and location of such groups in the compounds described herein as would be readily understood by the skilled person.

“Alkyl” is a straight chain or branched saturated aliphatic hydrocarbon group. In certain aspects, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-C₆alkyl is used herein in conjunction with another group, for example (C₃-C₇cycloalkyl)C₀-C₄alkyl, or —C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one aspect, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one aspect, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one aspect, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one aspect, the alkynyl group is optionally substituted as described herein.

“Alkoxy” is an alkyl group as defined above covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one aspect, the alkoxy group is optionally substituted as described herein.

“Alkanoyl” is an alkyl group as defined above covalently bound through a carbonyl (C═O) bridge. The carbonyl carbon is included in the number of carbons, for example C₂alkanoyl is a CH₃(C═O)— group. In one aspect, the alkanoyl group is optionally substituted as described herein.

“Halo” or “halogen” indicates, independently, any of fluoro, chloro, bromo or iodo.

“Aryl” indicates an aromatic group containing only carbon in the aromatic ring or rings. In one aspect, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one aspect, aryl groups are pendant. An example of a pendant ring is a phenyl group substituted with a phenyl group. In one aspect, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl.

Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring which contains from 1 to 4, or in some aspects 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some aspects from 1 to 3 or from 1 to 2, heteroatoms selected from N, O, S, B, or P, with remaining ring atoms being carbon. In one aspects, the only heteroatom is nitrogen. In one aspect, the only heteroatom is oxygen. In one aspect, the only heteroatom is sulfur. Monocyclic heteroaryl groups typically have from 5 to 6 ring atoms. In some aspects, bicyclic heteroaryl groups are 8- to 10-membered heteroaryl groups, that is groups containing 8 or 10 ring atoms in which one 5-, 6-, or 7-membered aromatic ring which contains from 1 to 4 heteroatoms selected from N, O, S, B, or P is fused to a second aromatic or non-aromatic ring, wherein the point of attachment is an aromatic ring. When the total number of S and O atoms in the heteroaryl ring exceeds 1, these heteroatoms are not adjacent to one another within the ring. In one aspect, the total number of S and O atoms in the heteroaryl ring is not more than 2. In another aspect, the total number of S and O atoms in the heteroaryl ring is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produced the same counterion. Lists of additional suitable salts may be found, e.g., in Remington's Pharmaceutical Sciences, 17^{th} ed., Mack Publishing Company, Easton, PA., p. 1418 (1985).

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

### Compounds of Formula I and Formula II

The present disclosure provides compounds capable of degrading son of sevenless homolog 1 (SOS1) via the ubiquitin-proteosome pathway. These compounds are useful in the treatment of medical disorders, for example cancers.

In one aspect, a compound is provided of Formula I or Formula II

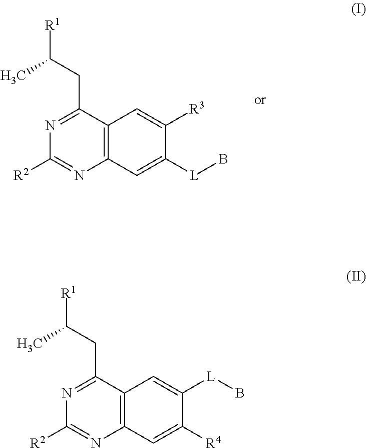

or a pharmaceutically acceptable salt or derivative thereof,
wherein:
R¹ is aryl optionally substituted with one or more groups independently selected from halo, amino, C₁-C₆ alkyl, and C₁-C₆ haloalkyl;
R² is selected from H or C₁-C₆ alkyl;
R³ and R⁴ are each C₁-C₆ alkoxy;
L is a linker, and
B is an E3 ubiquitin ligase-recruiting moiety.

In some aspects of Formula I or Formula II, R¹ is unsubstituted phenyl. In some aspects of Formula I or Formula II, R¹ is phenyl substituted with C₁-C₆ haloalkyl. In some aspects of Formula I or Formula II, R¹ is phenyl substituted with trifluoromethyl. In some aspects of Formula I or Formula II, R¹ is phenyl substituted with amino and C₁-C₆ haloalkyl. In some aspects of Formula I or Formula II R¹ is phenyl substituted with amino and trifluoromethyl.

In some aspects of Formula I or Formula II, R¹ is selected from:

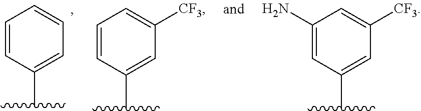

In some aspects of Formula I or Formula II, R² is H. In some aspects of Formula I or Formula II, R² is C₁-C₆ alkyl. In some aspects of Formula I or Formula I, R² is methyl.

In some aspects of Formula I, R³ is methoxy. In some aspects of Formula H, R⁴ is methoxy.

A Linker is included in the compounds of Formula I and Formula II. Linker is a chemically stable bivalent group that attaches B to the remaining structure of Formula I and Formula I. Linker as described herein can be used in either direction, i.e., either the left end is linked to B and the right end to the remaining structure of Formula I or Formula II, or the left end is linked to the remaining structure of Formula I and Formula II and the right end to B.

In some aspects, the Linker is a chain of 2 to 14, 15, 16, 17, 18, 19, or 20 or more carbon atoms, of which one or more carbons can be optionally replaced by a heteroatom such as O, N, S, or P.

In some aspects, the chain has 2, 3, 4, 5, 6, 7, 8, 9, 10, 11, 12, 13, 14, 15, 16, 18, 19, 19, or 20 contiguous atoms. For example, the chain may include 1 or more ethylene glycol units that can be contiguous, partially contiguous or non-contiguous (for example, 2, 3, 4, 5, 6, 7, 8, 9, 10, 11, or 12 ethylene glycol units).

In some aspects, the chain has at least 1, 2, 3, 4, 5, 6, 7, or 8 contiguous units which can be branched and which can be independently alkyl, aryl, heteroaryl, alkenyl, or alkynyl, cycloalkyl, or heterocycloalkyl substituents.

In some aspects, the linker can include or be comprised of one or more ethylene glycol, propylene glycol, lactic and/or glycolic acid units. Block and random lactic acid-co-glycolic acid moieties, as well as ethylene glycol and propylene glycol, are known in the art and can be modified to obtain the desired half-life and hydrophilicity. In certain aspects, these units can be flanked or interspersed with other moieties, such as for example alkyl, heteroalkyl, aryl, heteroaryl, cycloalkyl, heterocycloalkyl, etc., as desired to achieve the appropriate properties.

In some aspects, the Linker is an optionally substituted (poly)ethylene glycol having at least 1, at least 2, at least 3, at least 4, at least 5, at least 6, at least 7, at least 8, at least 9, at least 10, or more, ethylene glycol units, or optionally substituted alkyl groups interspersed with optionally substituted O, N, S, P or Si atoms.

In some aspects, the Linker is flanked, substituted, or interspersed with an alkyl, cycloalkyl, heterocycloalkyl, aryl, or heteroaryl group.

In some aspects, the Linker may be asymmetric or symmetric.

In some aspects, the Linker can be a non-linear chain, and can be, or include, cycloalkyl, heterocycloalkyl, aryl, or heteroaryl cyclic moieties.

In some aspects, the Linker is selected from L1:

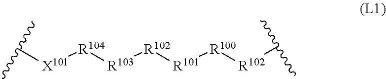

In some aspects, the Linker is selected from the group consisting of a moiety of Formula L1, Formula L2, Formula L3, Formula L4, Formula L5, Formula L6, Formula L7, Formula L8, Formula L9, or Formula L10:

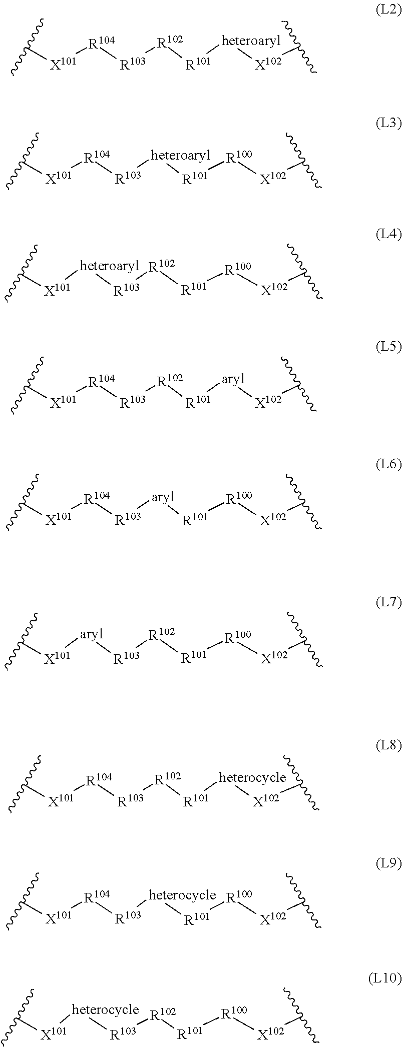

wherein:
X¹⁰¹ and X¹⁰² are independently at each occurrence selected from a bond, aryl, heteroaryl, cycloalkyl, heterocycle, NR¹³⁰, C(R¹³⁰)₂, O, C(O), and S;
R¹⁰⁰, R¹⁰¹, R¹⁰², R¹⁰³, and R¹⁰ are independently at each occurrence selected from the group consisting of a bond, alkyl, —C(O)—, —C(O)O—, —OC(O)—, —SO₂—, —S(O)—, C(S)—, —C(O)NR¹³⁰—, —NR¹³⁰C(O)—, —O—, —S—, —NR¹³⁰—, —C(R¹³⁰R¹³⁰)—, —P(O)(OR¹⁰⁶))—, —R(O)(OR¹⁰⁶)—, alkenyl, alkynyl, haloalkyl, alkoxy, aryl, heterocycloalkyl, cycloalkyl, heteroaryl, lactic acid, or glycolic acid, each of which may be optionally substituted with one or more (for example, 1, 2, 3, or 4) substituents independently selected from R¹⁴;
R¹⁰⁶ is independently at each occurrence selected from the group consisting of hydrogen, alkyl, arylalkyl, heteroarylalkyl, alkenyl, alkynyl, aryl, heteroaryl, cycloalkyl, or heterocycloalkyl;
R¹³⁰ is independently as each occurrence selected from the group consisting of hydrogen, alkyl, cycloalkyl, heterocycloalkyl, aryl, heteroaryl, —C(O)H, —C(O)OH, —C(O)alkyl, —C(O)Oalkyl, —C(O)(cycloalkyl, heterocycloalkyl, aryl, or heteroaryl), —C(O)O(cycloalkyl, heterocycloalkyl, aryl, or heteroaryl), alkenyl, or alkynyl; and
R¹⁴⁰ is independently at each occurrence selected from the group consisting of hydrogen, alkyl, alkenyl, alkynyl, fluoro, bromo, chloro, hydroxyl, alkoxy, azide, amino cyano, —NH(alkyl, cycloalkyl, heterocyloalkyl, aryl, or heteroaryl), —N(independently alkyl, cycloalkyl, heterocycloalkyl, aryl, or heteroaryl), —NHSO₂(alkyl, cycloalkyl, heterocycloalkyl, aryl, or heteroaryl), —N(alkyl, cycloalkyl, heterocycloalkyl, aryl, or heteroaryl)SO₂alkyl, —NHSO₂alkenyl, —N(alkyl)SO₂alkenyl, —NHSO₂alkynyl, —N(alkyl)SO₂alkynyl, haloalkyl, cycloalkyl, heterocycloalkyl, aryl, or heteroaryl.

In some aspects, R¹⁰⁰, R¹⁰¹, R¹⁰², R¹⁰³, and R¹⁰⁴ within the Linker are selected in such manner that: no two —C(═O)— moieties are adjected to each other; no two —O— or —NH-moieties are adjacent to each other, and/or no moieties are otherwise selected in an order such that an unstable molecule results (as defined as producing a molecule that has a shelf life at ambient temperature of less than about six months, five months, or four months) due to decomposition caused by the selection and order of R¹⁰⁰, R¹⁰¹, R¹⁰², R¹⁰³, and R¹⁰⁴.

The following are non-limiting examples of Linkers and/or moieties which comprise Linkers in whole or in part that can be used in this invention. Based on this elaboration, those of skill in the art will understand how to use the full breadth of Linkers that will accomplish the goal of the invention.

Non-limiting examples of moieties which may comprise the Linker, either in whole or in part, include, but are not limited to: a bond; —C(═O)—; —C≡C—; —NH—; —N(CH₃)—; —O—; —CH₂—; —(CH₂)₂—; —(CH₂)₃—; —(CH₂)₄—; —(CH₂)₅—; —(CH₂)₆—; —(CH₂)₇—; —(CH₂)₈—; —(CH₂)₉—; —(CH₂)₁₀—; —NH(C═O)—; —C(═O)NH—; —C(═O)CH₂—; —C(═O)(CH₂)₂—; —C(═O)(CH₂)₃—; —C(═O)(CH₂)₄—; —C(═O)(CH₂)₅—; —C(═O)(CH₂)₆—; —CH₂C(═O)—; —(CH₂)₂C(═O)—; —(CH₂)₃C(═O)—; —(CH₂)₄C(═O)—; —(CH₂)₅C(═O)—; —(CH₂)₆C(═O)—; —CH₂NH—; —(CH₂)₂NH—; —(CH₂)₃NH—; —(CH₂)₄NH—; —(CH₂)₅NH—; —(CH₂)₆NH—; —NHCH₂—; —NH(CH₂)₂—; —NH(CH₂)₃—; —NH(CH₂)₄—; —NH(CH₂)₅—; —NH(CH₂)₆—; —CH₂O—; —(CH₂)₂O—; —(CH₂)₃O—; —(CH₂)₄O—; —(CH₂)₅O—; —(CH₂)₆O—; —OCH₂—; —O(CH₂)₂—; —O(CH₂)₃—; —O(CH₂)₄—; —O(CH₂)₅—; —O(CH₂)₆—;

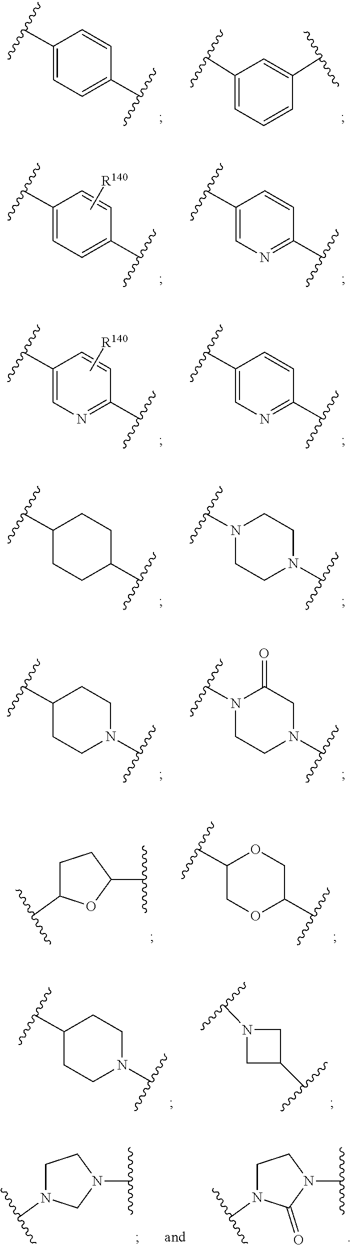

Further non-limiting examples of moieties which may comprise the Linker, either in whole or in part, include but are not limited to:

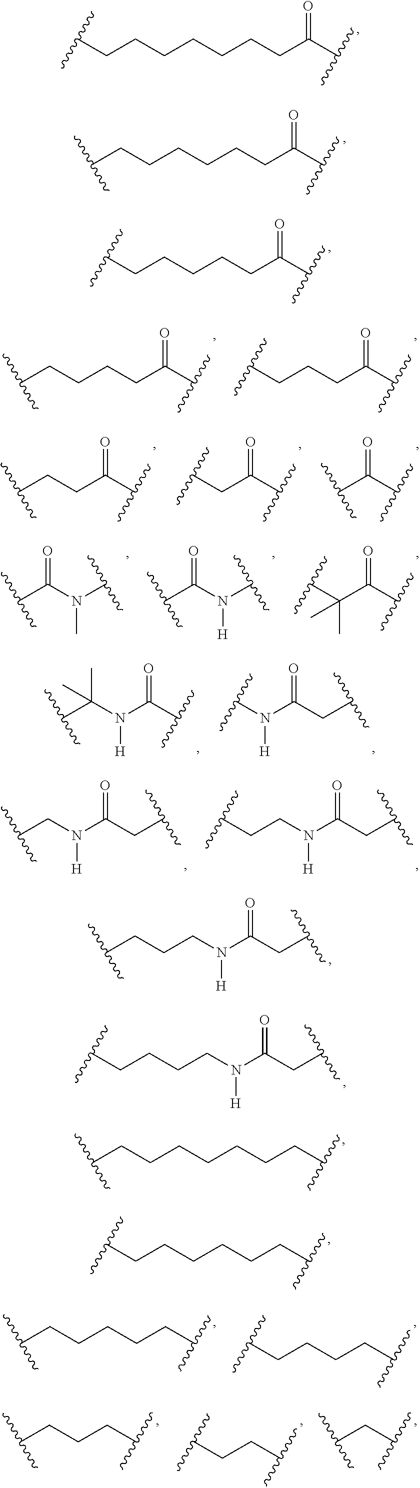

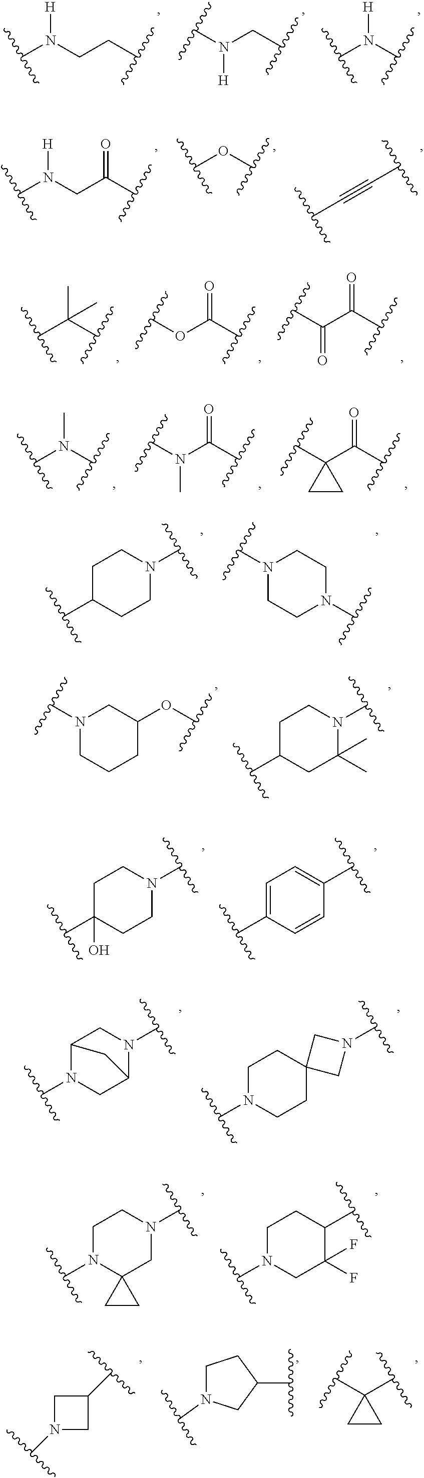

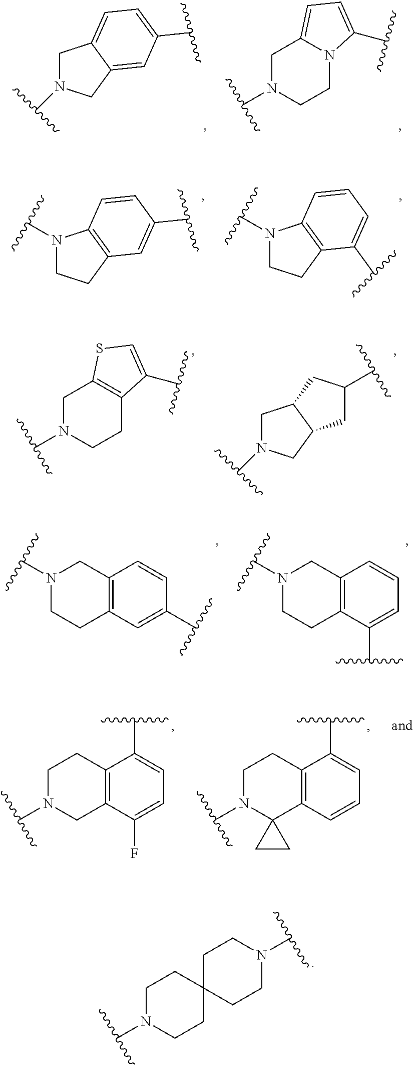

In some aspects, the Linker may comprise, either in whole or in part, a moiety selected from:

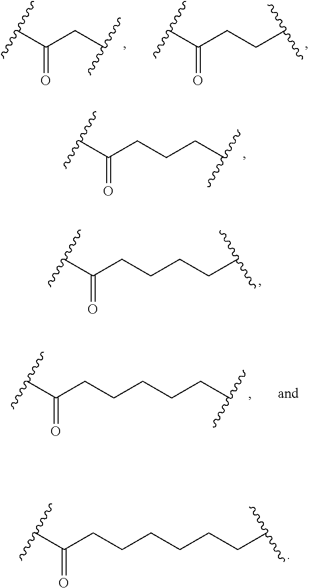 [SMILES: *CC(*)=O ; *CCC(*)=O ; *CCCC(*)=O ; *CCCCC(*)=O ; *CCCCCC(*)=O ; *CCCCCCC(*)=O]

In some aspects, the Linker may comprise, either in whole or in part, a moiety selected from:

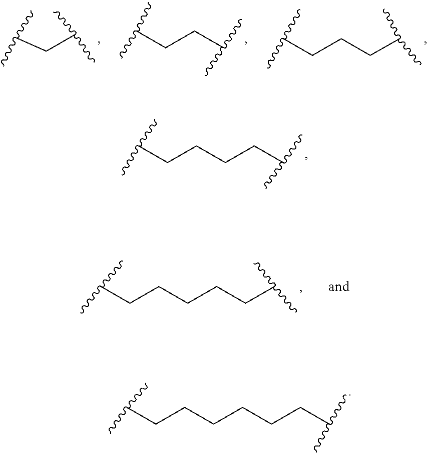

In some aspects, the Linker may comprise, either in whole or in part, a moiety selected from:

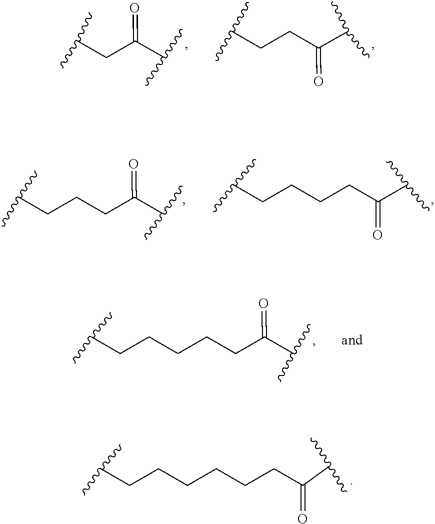 [SMILES: *CC(*)=O ; *CCC(*)=O ; *CCCC(*)=O ; *CCCCC(*)=O ; *CCCCCC(*)=O ; *CCCCCCC(*)=O]

In some aspects, the Linker may comprise, either in whole or in part,

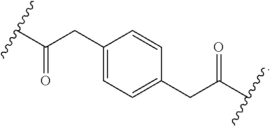 [SMILES: *C(=O)Cc1ccc(CC(*)=O)cc1]

In some aspects, the Linker may comprise, either in whole or in part,

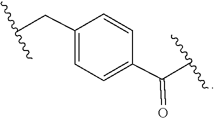 [SMILES: *Cc1ccc(C(*)=O)cc1]

In some aspects, the Linker may comprise, either in whole or in part, a moiety selected from:

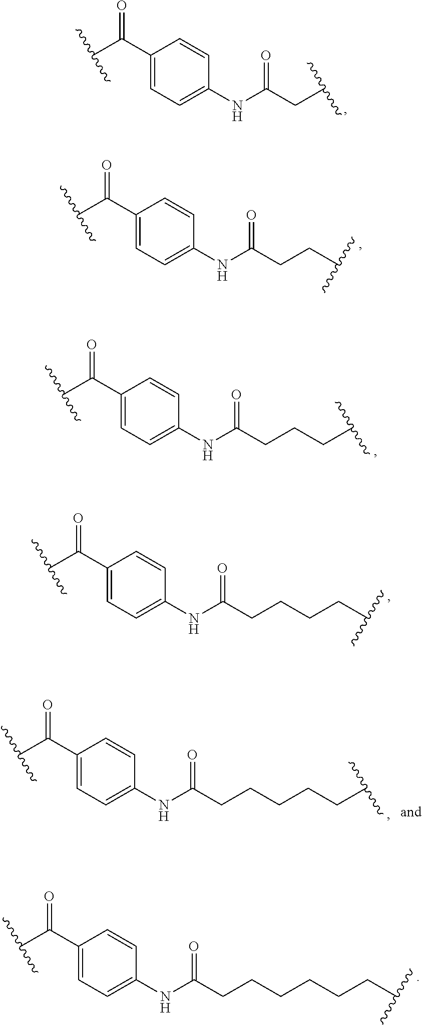 [SMILES: *CC(=O)Nc1ccc(C(*)=O)cc1 ; *CCC(=O)Nc1ccc(C(*)=O)cc1 ; *CCCC(=O)Nc1ccc(C(*)=O)cc1 ; *CCCCC(=O)Nc1ccc(C(*)=O)cc1 ; *CCCCCC(=O)Nc1ccc(C(*)=O)cc1 ; *CCCCCCC(=O)Nc1ccc(C(*)=O)cc1]

In some aspects, the Linker may comprise, either in whole or in part, a moiety selected from:

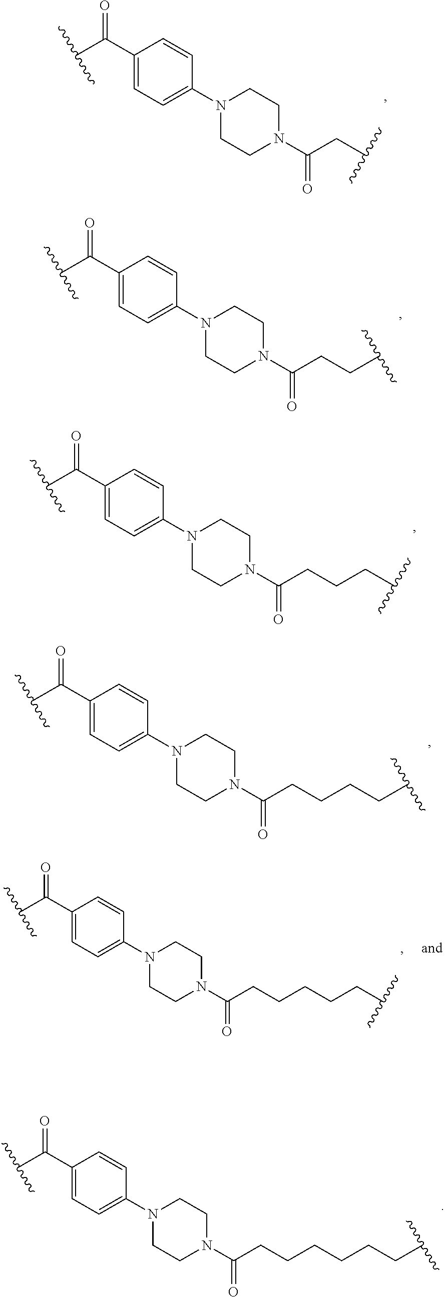 [SMILES: *CC(=O)N1CCN(c2ccc(C(*)=O)cc2)CC1 ; *CCC(=O)N1CCN(c2ccc(C(*)=O)cc2)CC1 ; *CCCC(=O)N1CCN(c2ccc(C(*)=O)cc2)CC1 ; *CCCCC(=O)N1CCN(c2ccc(C(*)=O)cc2)CC1 ; *CCCCCC(=O)N1CCN(c2ccc(C(*)=O)cc2)CC1 ; *CCCCCCC(=O)N1CCN(c2ccc(C(*)=O)cc2)CC1]

In some aspects, the Linker may comprise, either in whole or in part, a moiety selected from:

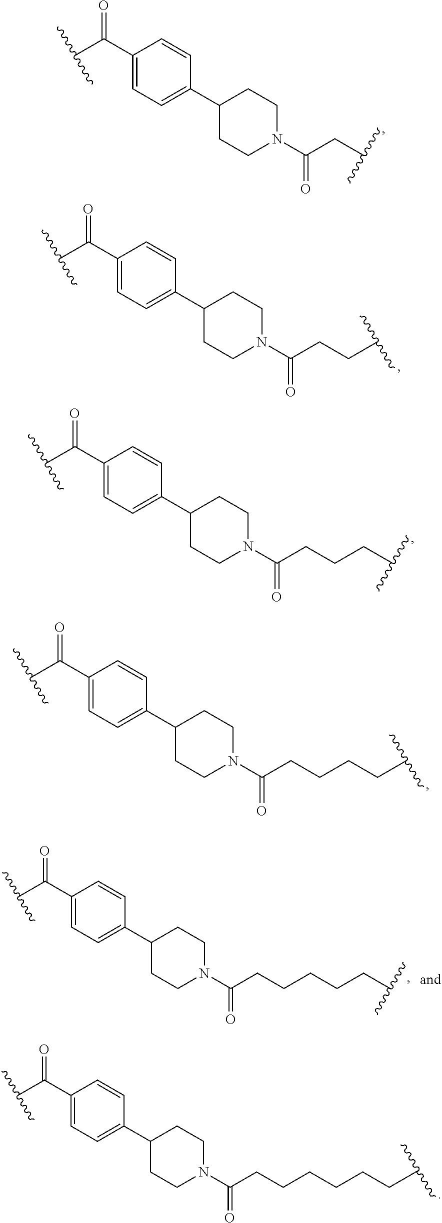 [SMILES: *CC(=O)N1CCC(c2ccc(C(*)=O)cc2)CC1 ; *CCC(=O)N1CCC(c2ccc(C(*)=O)cc2)CC1 ; *CCCC(=O)N1CCC(c2ccc(C(*)=O)cc2)CC1 ; *CCCCC(=O)N1CCC(c2ccc(C(*)=O)cc2)CC1 ; *CCCCCC(=O)N1CCC(c2ccc(C(*)=O)cc2)CC1 ; *CCCCCCC(=O)N1CCC(c2ccc(C(*)=O)cc2)CC1]

In some aspects, the Linker may comprise, either in whole or in part, a moiety selected from:

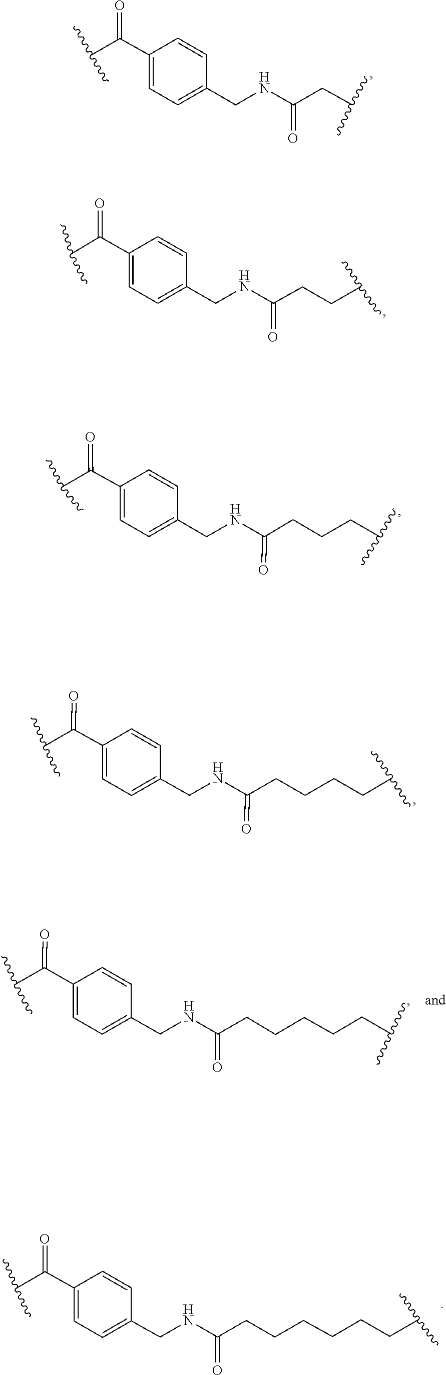 [SMILES: *CC(=O)NCc1ccc(C(*)=O)cc1 ; *CCC(=O)NCc1ccc(C(*)=O)cc1 ; *CCCC(=O)NCc1ccc(C(*)=O)cc1 ; *CCCCC(=O)NCc1ccc(C(*)=O)cc1 ; *CCCCCC(=O)NCc1ccc(C(*)=O)cc1 ; *CCCCCCC(=O)NCc1ccc(C(*)=O)cc1]

In Some aspects, the Linker may comprise, either in whole or in part, a moiety selected from:

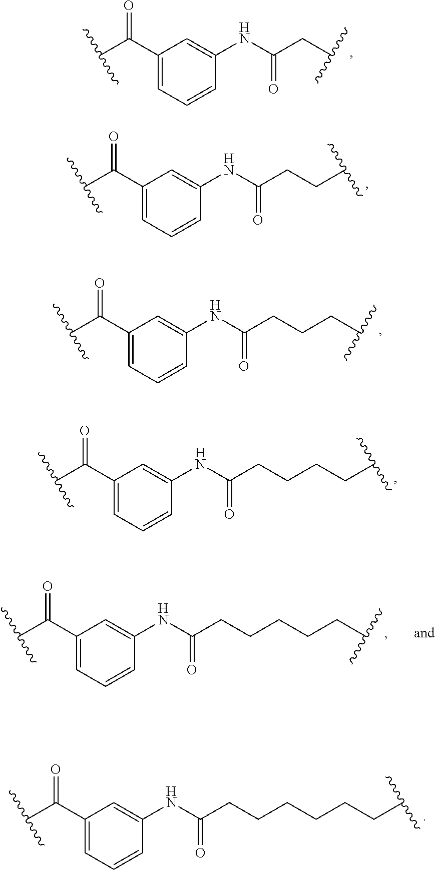 [SMILES: *CC(=O)Nc1cccc(C(*)=O)c1 ; *CCC(=O)Nc1cccc(C(*)=O)c1 ; *CCCC(=O)Nc1cccc(C(*)=O)c1 ; *CCCCC(=O)Nc1cccc(C(*)=O)c1 ; *CCCCCC(=O)Nc1cccc(C(*)=O)c1 ; *CCCCCCC(=O)Nc1cccc(C(*)=O)c1]

In some aspects, the Linker may comprise, either in whole or in part, a moiety selected from:

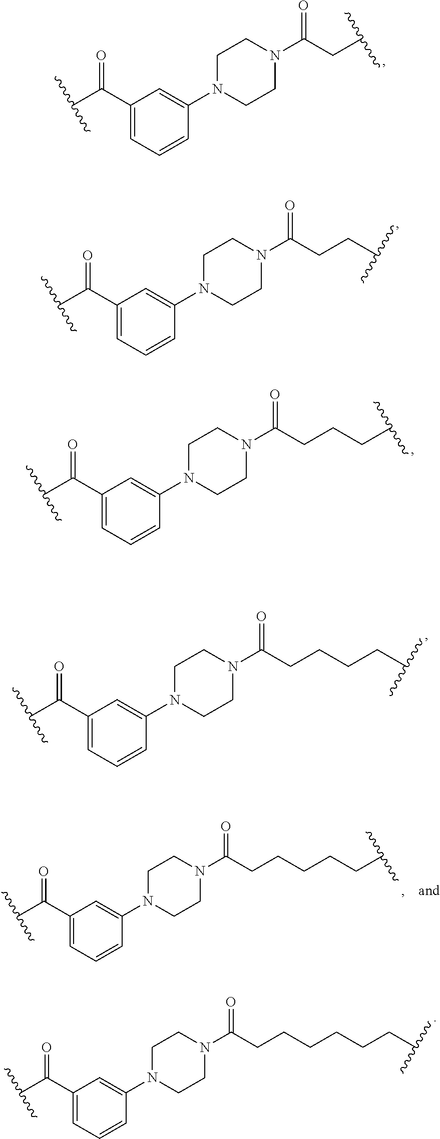 [SMILES: *CC(=O)N1CCN(c2cccc(C(*)=O)c2)CC1 ; *CCC(=O)N1CCN(c2cccc(C(*)=O)c2)CC1 ; *CCCC(=O)N1CCN(c2cccc(C(*)=O)c2)CC1 ; *CCCCC(=O)N1CCN(c2cccc(C(*)=O)c2)CC1 ; *CCCCCC(=O)N1CCN(c2cccc(C(*)=O)c2)CC1 ; *CCCCCCC(=O)N1CCN(c2cccc(C(*)=O)c2)CC1]

In some aspects, the Linker may comprise, either in whole or in part, a moiety selected from:

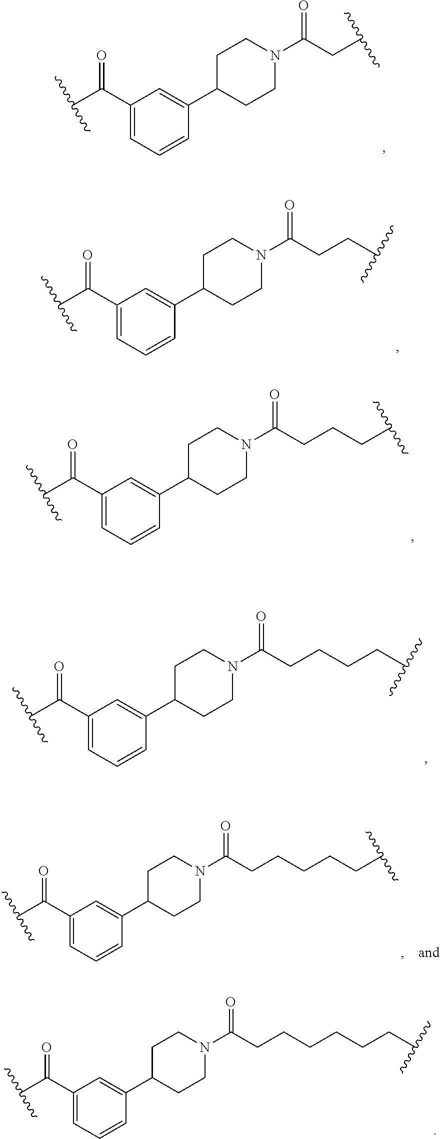 [SMILES: *CC(=O)N1CCC(c2cccc(C(*)=O)c2)CC1 ; *CCC(=O)N1CCC(c2cccc(C(*)=O)c2)CC1 ; *CCCC(=O)N1CCC(c2cccc(C(*)=O)c2)CC1 ; *CCCCC(=O)N1CCC(c2cccc(C(*)=O)c2)CC1 ; *CCCCCC(=O)N1CCC(c2cccc(C(*)=O)c2)CC1 ; *CCCCCCC(=O)N1CCC(c2cccc(C(*)=O)c2)CC1]

In some aspects, the Linker may comprise, either in whole or in part, a moiety selected from:

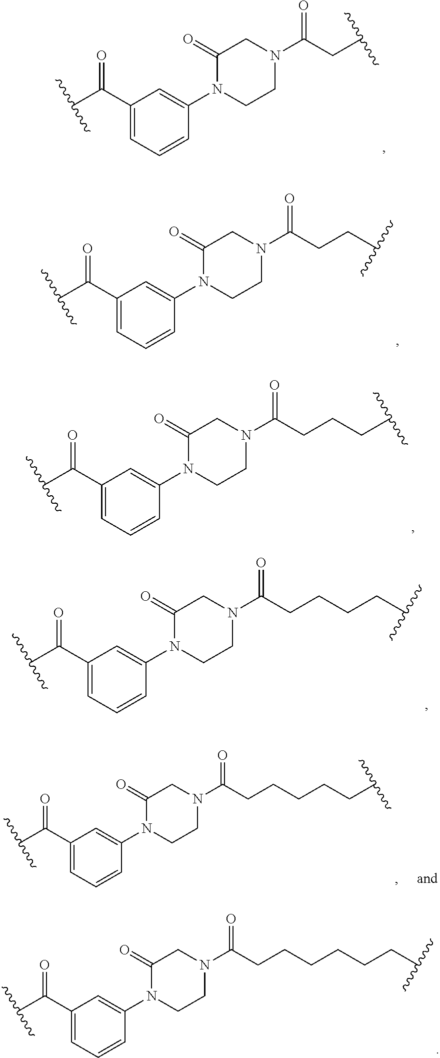 [SMILES: *CC(=O)N1CCN(c2cccc(C(*)=O)c2)C(=O)C1 ; *CCC(=O)N1CCN(c2cccc(C(*)=O)c2)C(=O)C1 ; *CCCC(=O)N1CCN(c2cccc(C(*)=O)c2)C(=O)C1 ; *CCCCC(=O)N1CCN(c2cccc(C(*)=O)c2)C(=O)C1 ; *CCCCCC(=O)N1CCN(c2cccc(C(*)=O)c2)C(=O)C1 ; *CCCCCCC(=O)N1CCN(c2cccc(C(*)=O)c2)C(=O)C1]

In some aspects, the Linker may comprise, either in whole or in part, a moiety selected from:

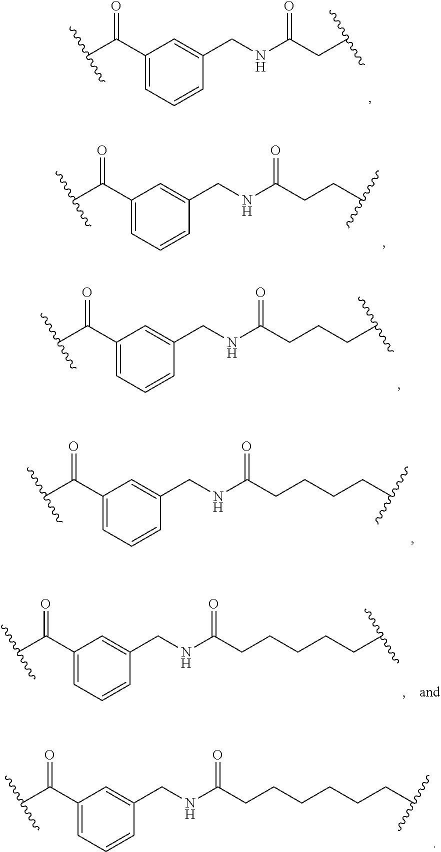 [SMILES: *CC(=O)NCc1cccc(C(*)=O)c1 ; *CCC(=O)NCc1cccc(C(*)=O)c1 ; *CCCC(=O)NCc1cccc(C(*)=O)c1 ; *CCCCC(=O)NCc1cccc(C(*)=O)c1 ; *CCCCCC(=O)NCc1cccc(C(*)=O)c1 ; *CCCCCCC(=O)NCc1cccc(C(*)=O)c1]

In some aspects, the Linker may comprise, either in whole or in part, a moiety selected from:

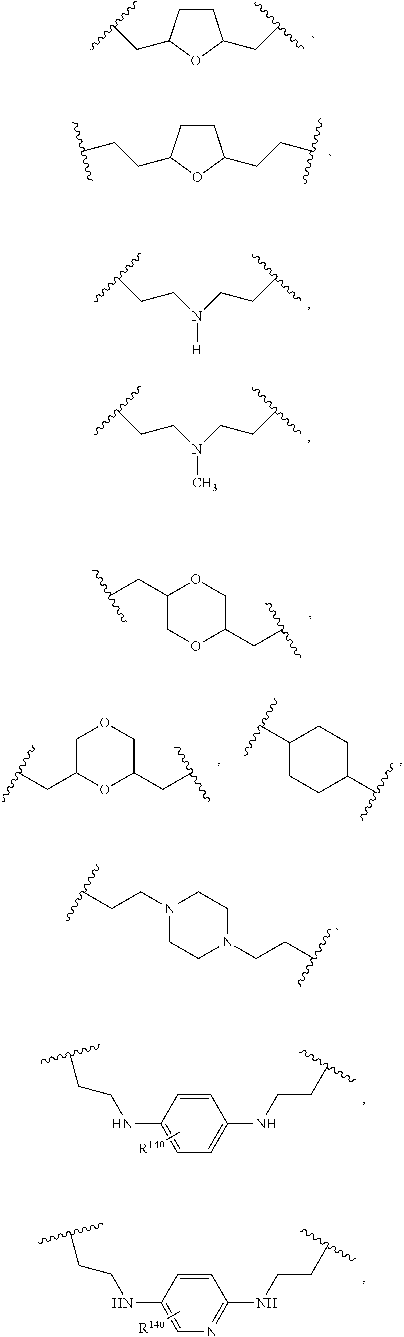

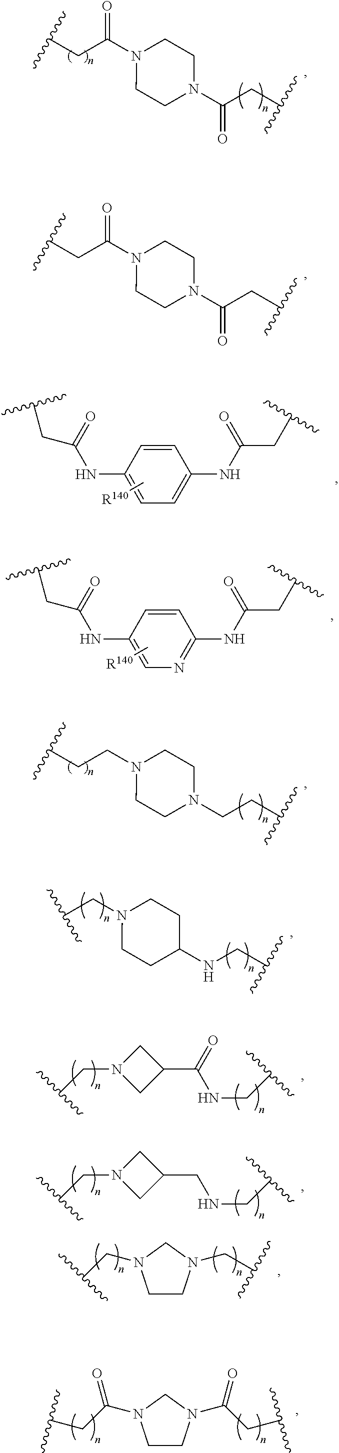

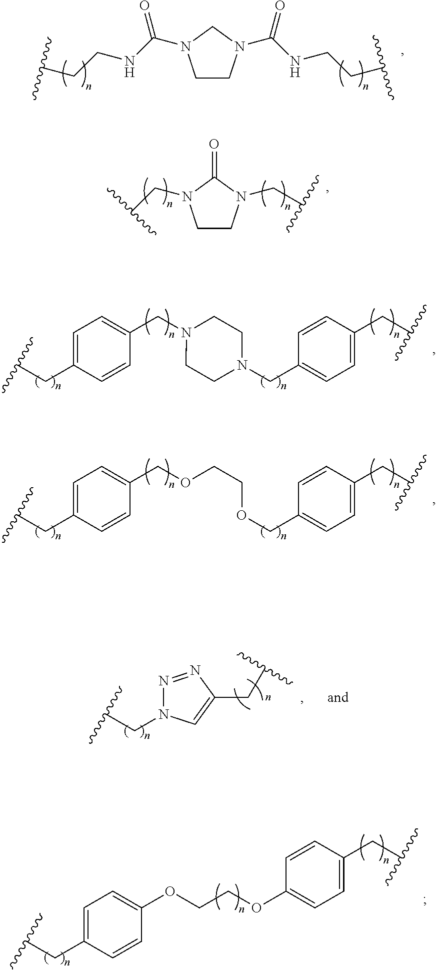

wherein n is independently selected at each occurrence from 1, 2, 3, 4, 5, and 6; and all other variables are as defined herein.

In some aspects, the Linker may comprise, either in whole or in part, a moiety selected from:

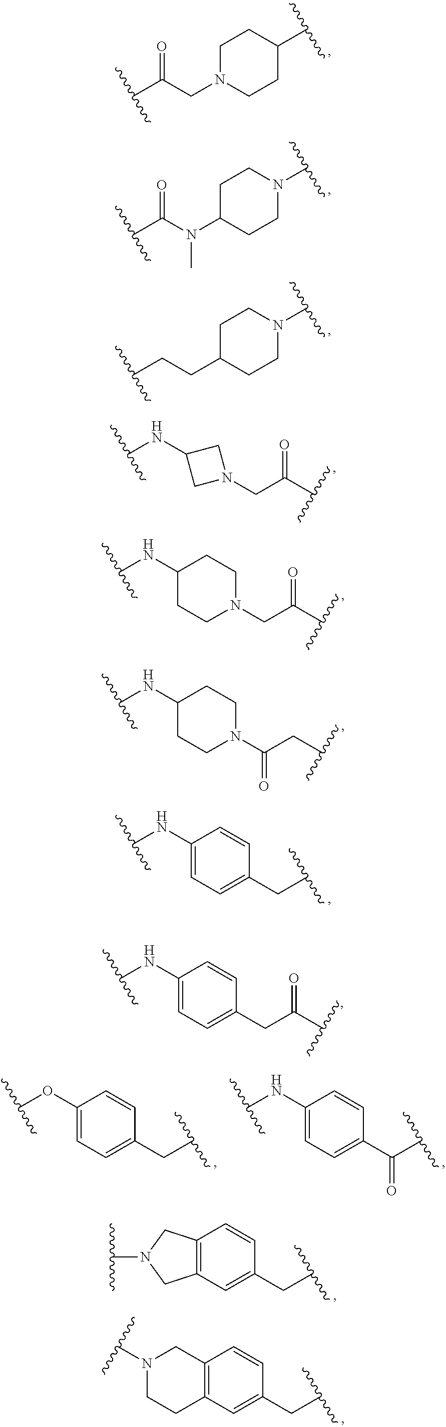

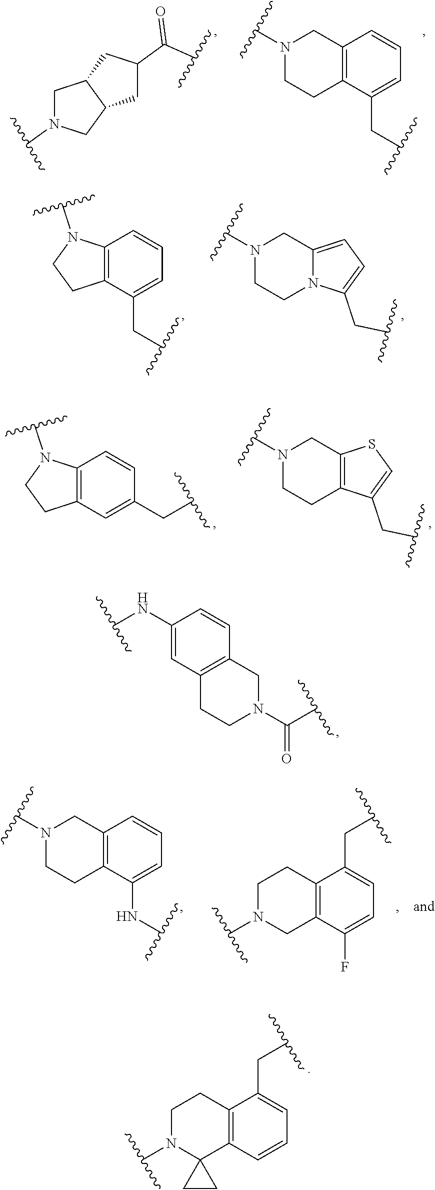

In some aspects, the Linker may comprise, either in whole or in part, a moiety selected from:

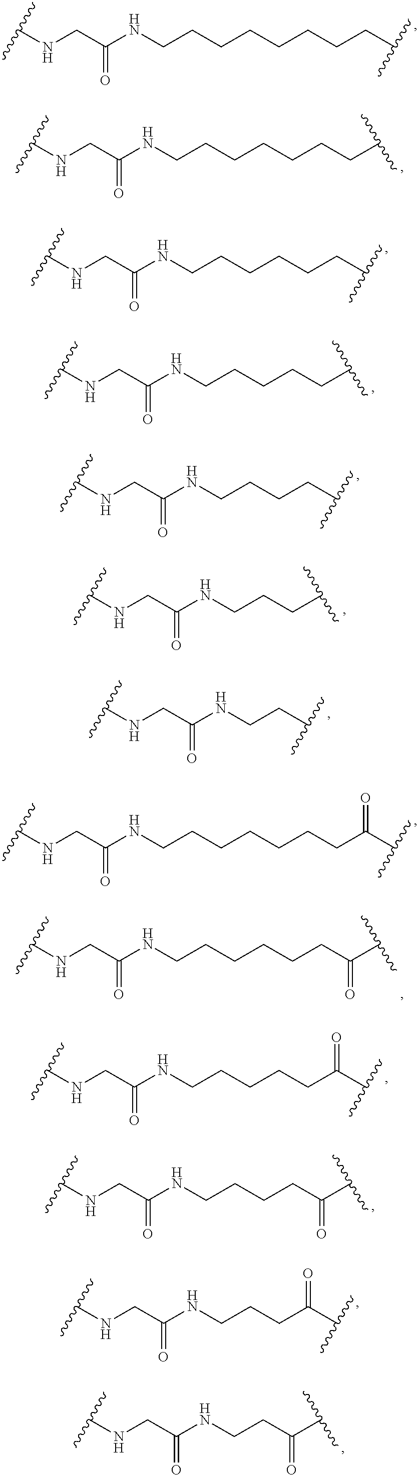

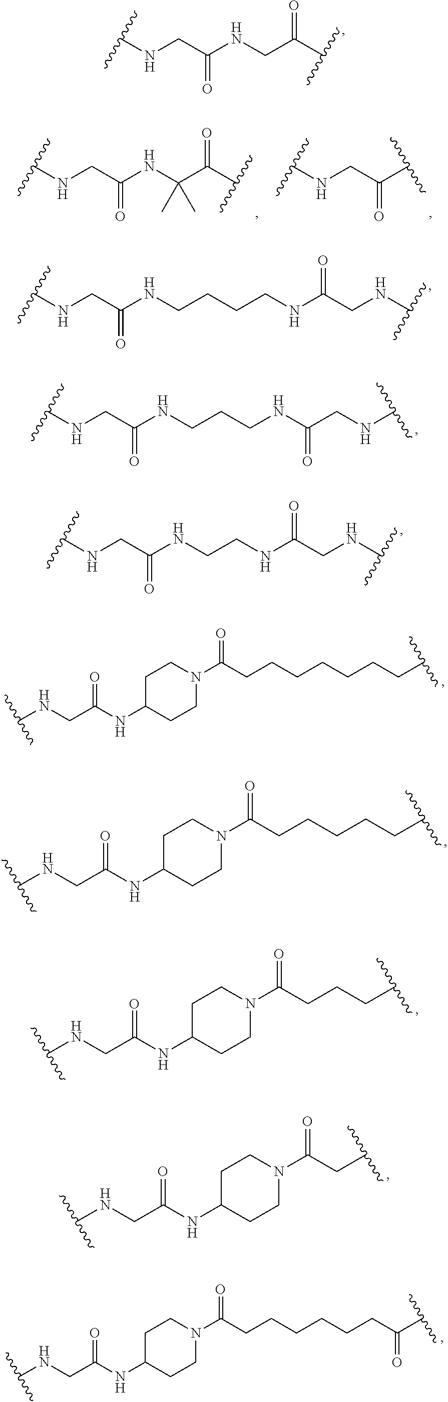

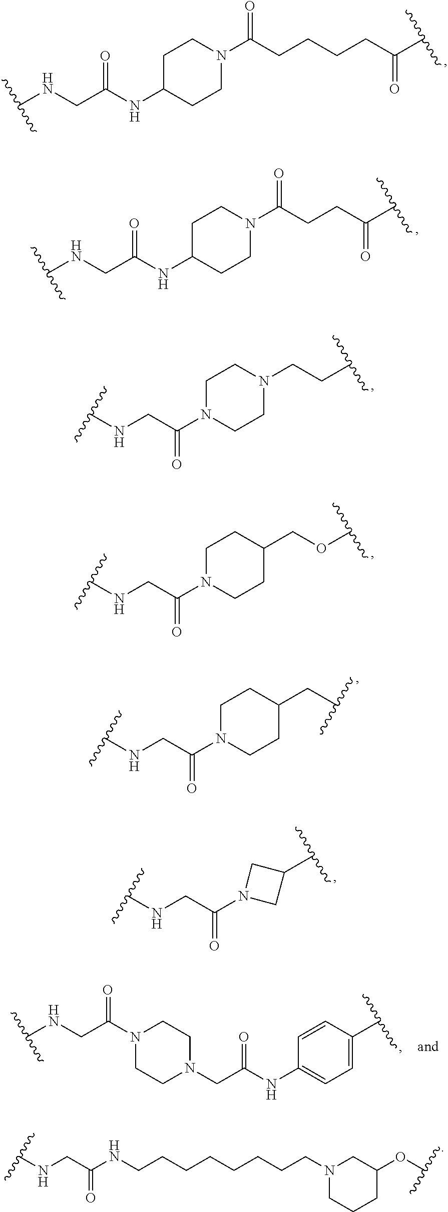

In some aspects, the Linker may comprise, either in whole or in part, a moiety selected from:

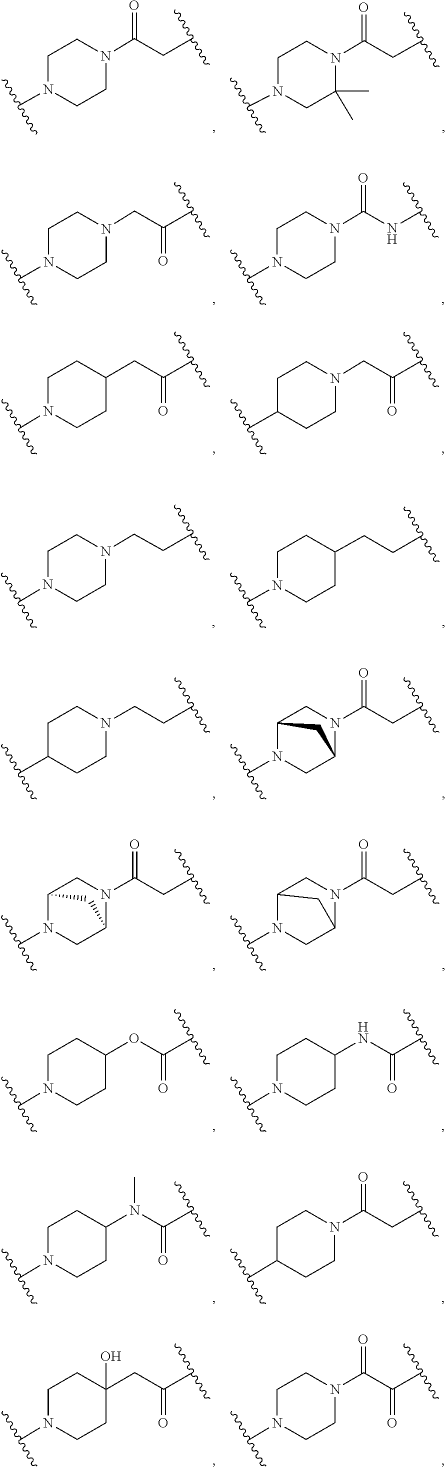

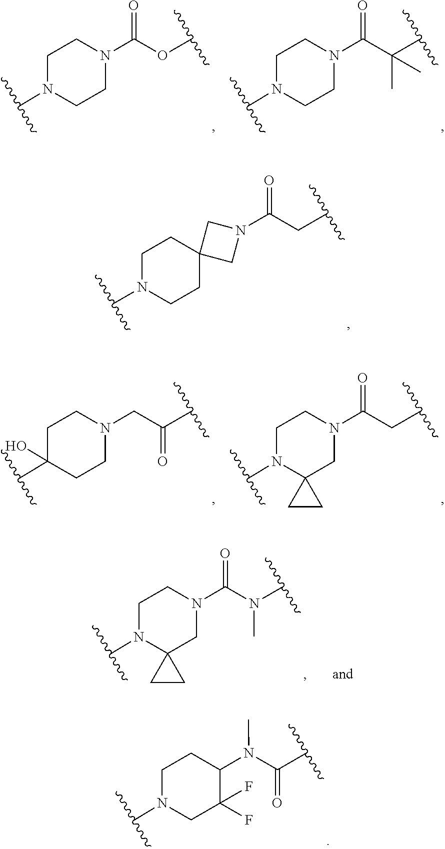

In some aspects, the Linker may comprise, either in whole or in part, a moiety selected from:

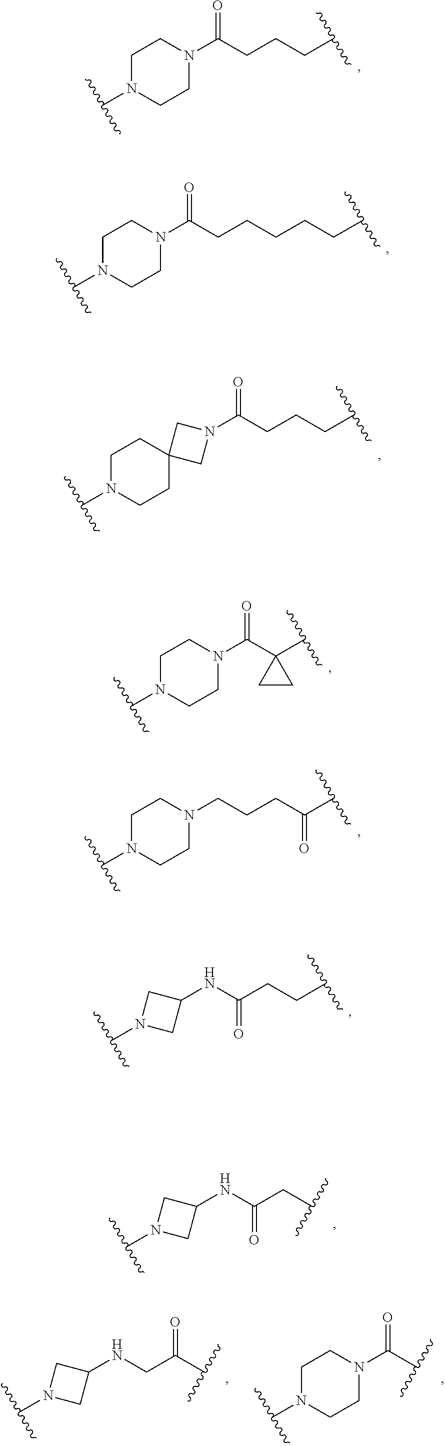

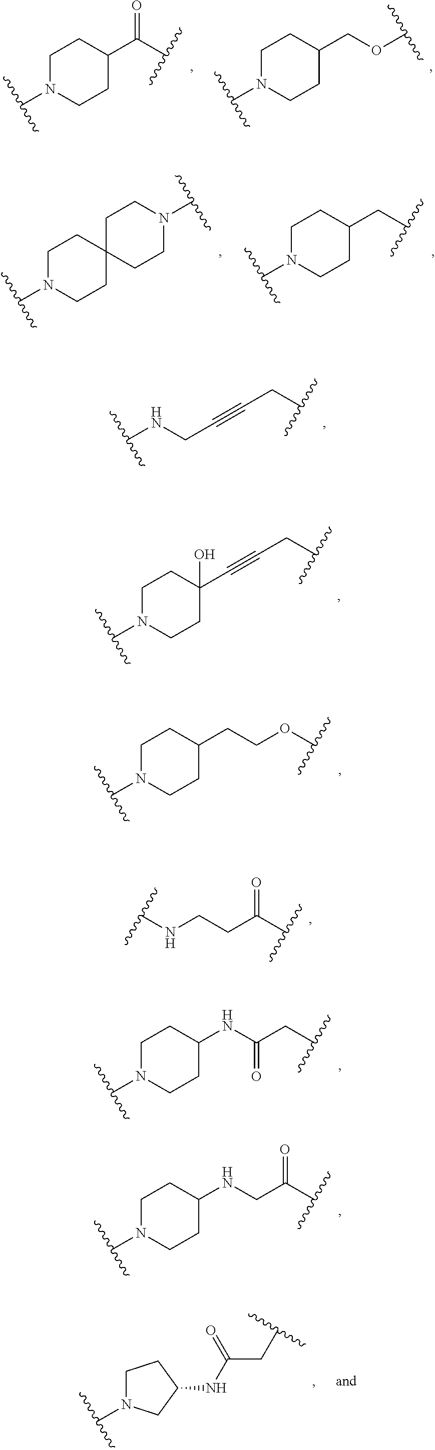

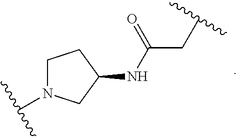 [SMILES: *CC(=O)N[C@@H]1CCN(*)C1]

In some aspects, the Linker may comprise, either in whole or in part, a moiety selected from:

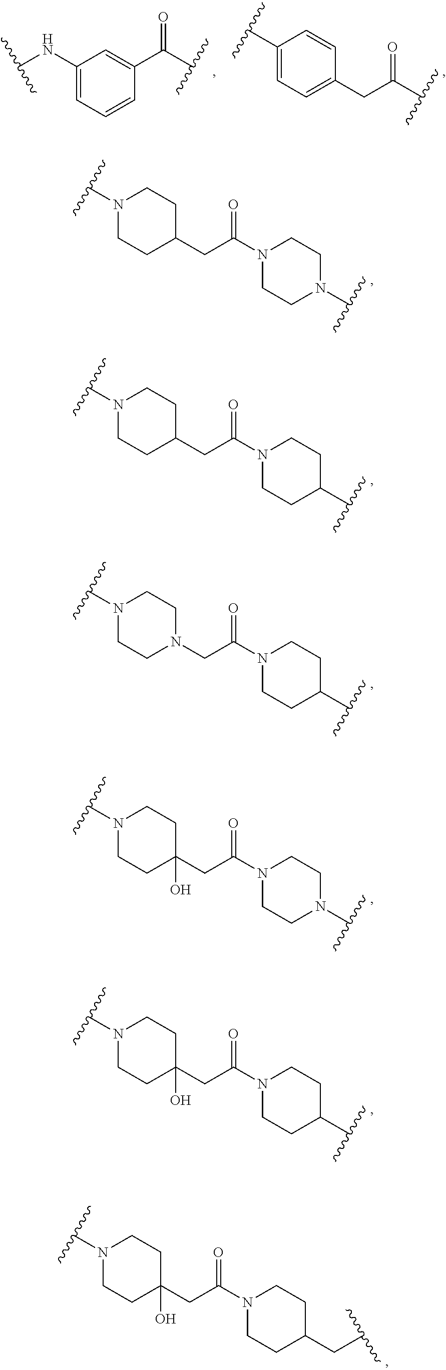

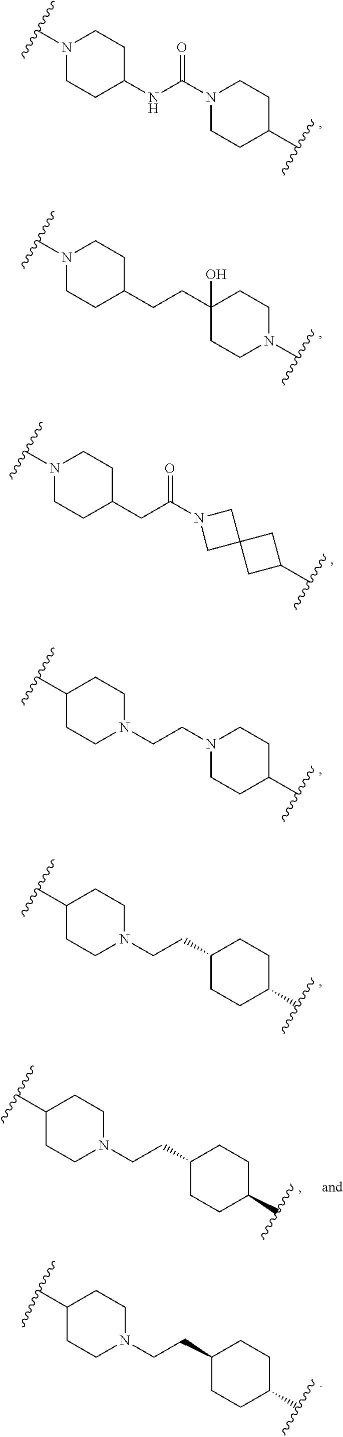

The E3 ubiquitin ligase-recruiting moiety B is a chemical moiety capable of recruiting an E3 ubiquitin ligase to a given substrate protein (for example, TAF1) resulting in its targeted degradation. In some aspects, B is a chemical moiety based upon a high affinity small molecule for E3 ubiquitin ligases, such as von Hippel-Lindau or cereblon. In some aspects, B is a chemical moiety based upon von Hippel-Lindau binder such as VH032 or VH298. In some aspects, B is a chemical moiety based upon a cereblon binder such as thalidomide, lenalidomide, or pomalidomide.

In some aspects of Formula I or Formula II, B is selected from

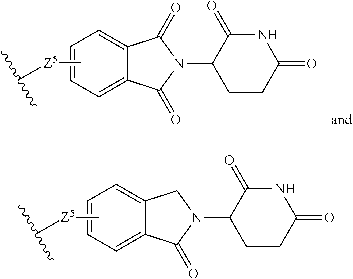

wherein Z⁵ is selected from O, N(R^{a}), and CH₂.

In some aspects of Formula I or Formula II, B is selected from

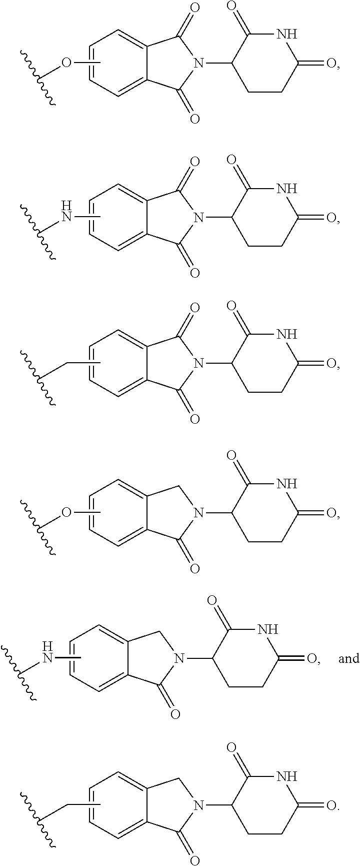

In some aspects of Formula I or Formula II, B is selected from

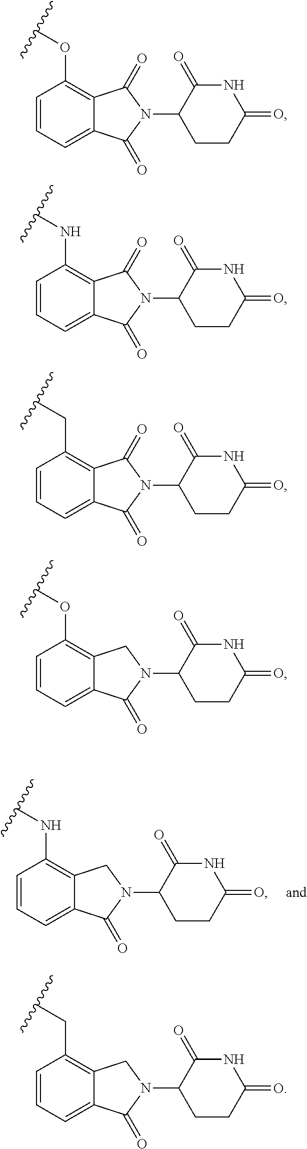

In some aspects of Formula I or Formula II, B is selected from

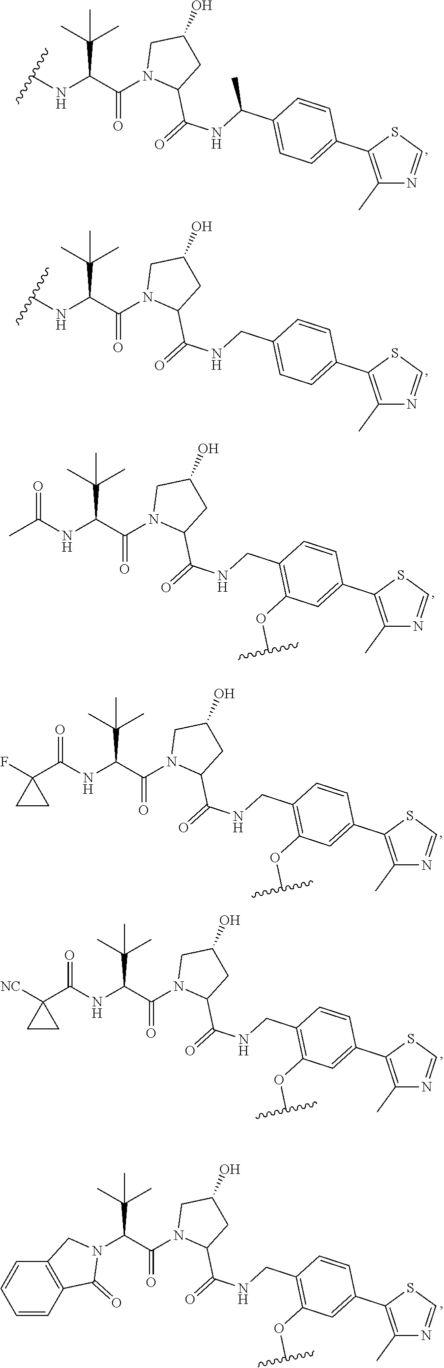 [SMILES: *N[C@H](C(=O)N1C[C@H](O)CC1C(=O)N[C@@H](C)c1ccc(-c2scnc2C)cc1)C(C)(C)C ; *N[C@H](C(=O)N1C[C@H](O)CC1C(=O)NCc1ccc(-c2scnc2C)cc1)C(C)(C)C ; *Oc1cc(-c2scnc2C)ccc1CNC(=O)C1C[C@@H](O)CN1C(=O)[C@@H](NC(C)=O)C(C)(C)C ; *Oc1cc(-c2scnc2C)ccc1CNC(=O)C1C[C@@H](O)CN1C(=O)[C@@H](NC(=O)C1(F)CC1)C(C)(C)C ; *Oc1cc(-c2scnc2C)ccc1CNC(=O)C1C[C@@H](O)CN1C(=O)[C@@H](NC(=O)C1(C#N)CC1)C(C)(C)C ; *Oc1cc(-c2scnc2C)ccc1CNC(=O)C1C[C@@H](O)CN1C(=O)[C@@H](N1Cc2ccccc2C1=O)C(C)(C)C]

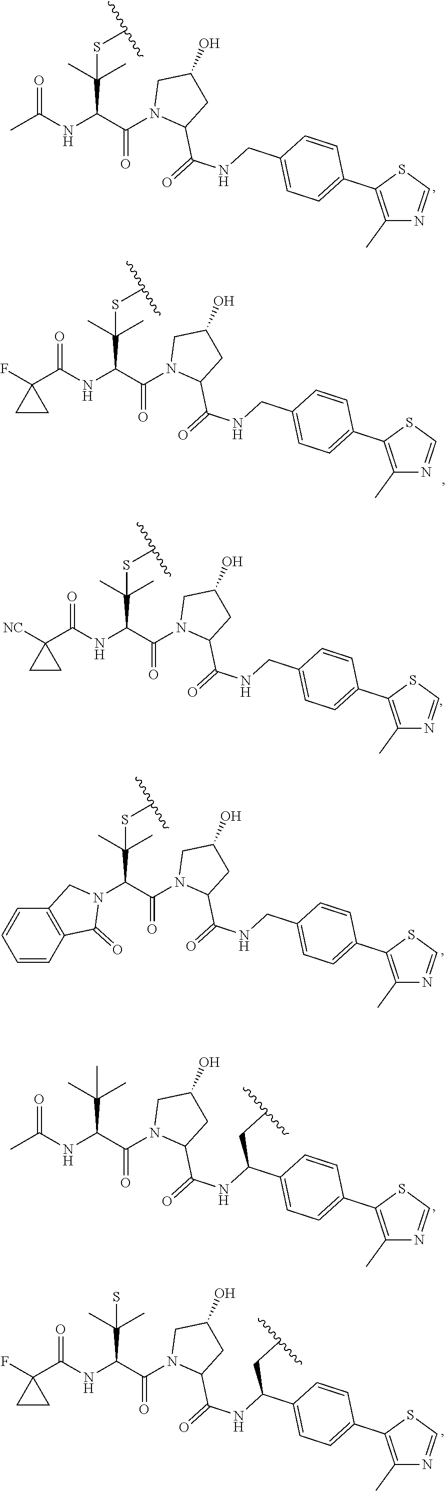 [SMILES: *SC(C)(C)[C@H](NC(C)=O)C(=O)N1C[C@H](O)CC1C(=O)NCc1ccc(-c2scnc2C)cc1 ; *SC(C)(C)[C@H](NC(=O)C1(F)CC1)C(=O)N1C[C@H](O)CC1C(=O)NCc1ccc(-c2scnc2C)cc1 ; *SC(C)(C)[C@H](NC(=O)C1(C#N)CC1)C(=O)N1C[C@H](O)CC1C(=O)NCc1ccc(-c2scnc2C)cc1 ; *SC(C)(C)[C@@H](C(=O)N1C[C@H](O)CC1C(=O)NCc1ccc(-c2scnc2C)cc1)N1Cc2ccccc2C1=O ; *C[C@H](NC(=O)C1C[C@@H](O)CN1C(=O)[C@@H](NC(C)=O)C(C)(C)C)c1ccc(-c2scnc2C)cc1 ; *C[C@H](NC(=O)C1C[C@@H](O)CN1C(=O)[C@@H](NC(=O)C1(F)CC1)C(C)(C)S)c1ccc(-c2scnc2C)cc1]

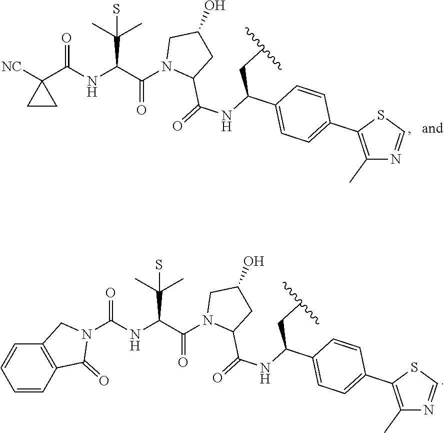 [SMILES: *C[C@H](NC(=O)C1C[C@@H](O)CN1C(=O)[C@@H](NC(=O)C1(C#N)CC1)C(C)(C)S)c1ccc(-c2scnc2C)cc1 ; *C[C@H](NC(=O)C1C[C@@H](O)CN1C(=O)[C@@H](NC(=O)N1Cc2ccccc2C1=O)C(C)(C)S)c1ccc(-c2scnc2C)cc1]

In some aspects, a compound is provided of Formula I and Formula II selected from:

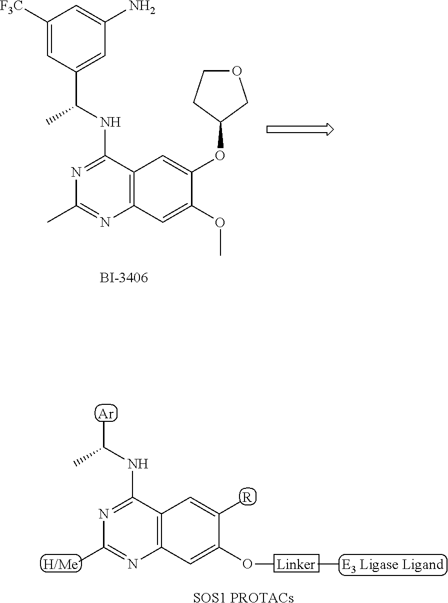

| | |
|---|---|
| 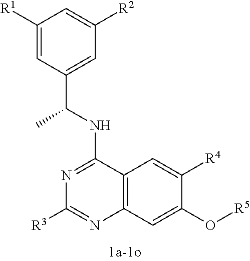 | 1a (P3): R¹ = H, R² = H, R³ = Me, R⁴ = OMe, R⁵ = LE1 1b (P4): R¹ = H, R² = CF₃, R³ = Me, R⁴ = OMe, R⁵ = LE1 1c (P5): R¹ = NH₂, R² = CF₃, R³ = Me, R⁴ = OMe, R⁵ = LE1 1d (P8): R¹ = NH₂, R² = CF₃, R³ = Me, R⁴ = OMe, R⁵ = LE2 1e (P9): R¹ = NH₂, R² = CF₃, R³ = Me, R⁴ = OMe, R⁵ = LE3 1f (P10): R¹ = NH₂, R² = CF₃, R³ = Me, R⁴ = OMe, R⁵ = LE4 1g (P7): R¹ = NH₂, R² = CF₃, R³ = Me, R⁴ = OMe, R⁵ = LE5 1h (P6): R¹ = NH₂, R² = CF₃, R³ = Me, R⁴ = OMe, R⁵ = LE6 1i (P2): R¹ = NH₂, R² = CF₃, R³ = Me, R⁴ = OMe, R⁵ = LE7 1j (P1): R¹ = H, R² = H, R³ = Me, R⁴ = OMe, R⁵ = LE7 1k (P11): R¹ = H, R² = H, R³ = H, R⁴ = OMe, R⁵ = LE8 1l (P12): R¹ = H, R² = CF₃, R³ = H, R⁴ = OMe, R⁵ = LE8 1m (P15): R¹ = H, R² = H, R³ = Me, R⁴ = OMe, R⁵ = LE8 1n (P13): R¹ = H, R² = CF₃, R³ = Me, R⁴ = LE9, R⁵ = OMe 1o (P14): R¹ = H, R² = CF₃, R³ = Me, R⁴ = LE10, R⁵ = OMe |

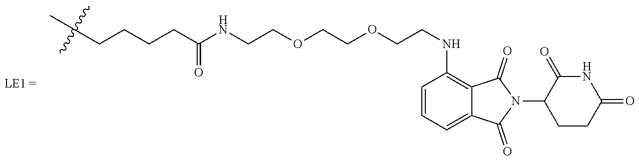 [SMILES: *CCCCC(=O)NCCOCCOCCNc1cccc2c1C(=O)N(C1CCC(=O)NC1=O)C2=O]

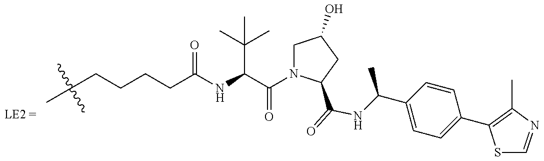 [SMILES: *CCCCC(=O)N[C@H](C(=O)N1C[C@H](O)C[C@H]1C(=O)N[C@@H](C)c1ccc(-c2scnc2C)cc1)C(C)(C)C]

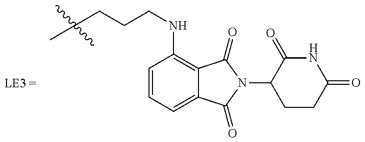 [SMILES: *CCCNc1cccc2c1C(=O)N(C1CCC(=O)NC1=O)C2=O]

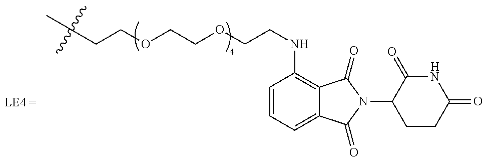

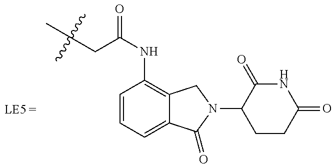 [SMILES: *CC(=O)Nc1cccc2c1CN(C1CCC(=O)NC1=O)C2=O]

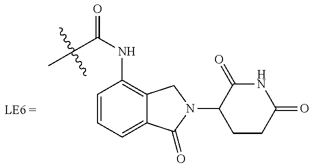 [SMILES: *C(=O)Nc1cccc2c1CN(C1CCC(=O)NC1=O)C2=O]

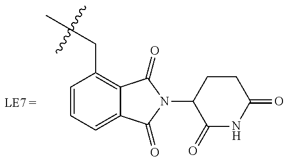 [SMILES: *Cc1cccc2c1C(=O)N(C1CCC(=O)NC1=O)C2=O]

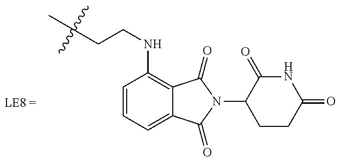 [SMILES: *CCNc1cccc2c1C(=O)N(C1CCC(=O)NC1=O)C2=O]

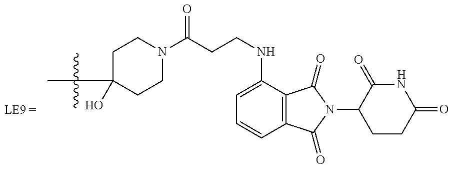 [SMILES: *C1(O)CCN(C(=O)CCNc2cccc3c2C(=O)N(C2CCC(=O)NC2=O)C3=O)CC1]

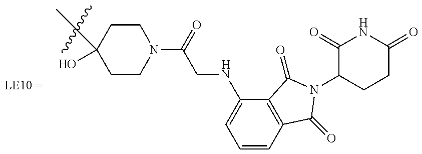 [SMILES: *C1(O)CCN(C(=O)CNc2cccc3c2C(=O)N(C2CCC(=O)NC2=O)C3=O)CC1]

The present disclosure also includes compounds of Formula I and Formula II with at least one desired isotopic substitution of an atom, at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P, ³²P, ³⁵S, ³⁶Cl, and ¹²⁵I, respectively. In one aspect, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (²H) and tritium (³H) may optionally be used anywhere in described structures that achieves the desired result. Alternatively or in addition, isotopes of carbon, e.g., ¹³C and ¹⁴C, may be used. In one aspect, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tmax, Cmax, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example deuterium substitutions, can be partial or complete. Partial deuterium substitution means that at least one hydrogen is substituted with deuterium. In certain aspects, the isotope is 80, 85, 90, 95, or 99% or more enriched in an isotope at any location of interest. In some aspects, deuterium is 80, 85, 90, 95, or 99% enriched at a desired location. Unless otherwise stated, the enrichment at any point is above natural abundance, and in an aspect is enough to alter a detectable property of the compounds as a drug in a human.

The compounds of the present disclosure may form a solvate with solvents (including water). Therefore, in one aspect, the invention includes a solvated form of the active compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a disclosed compound and water. Pharmaceutically acceptable solvates in accordance with the invention include those wherein the solvent of crystallization may be isotopically substituted, e.g., D₂O, d₆-acetone, or d₆-DMSO. A solvate can be in a liquid or solid form.

A “prodrug” as used herein means a compound which when administered to a host in vivo is converted into a parent drug. As used herein, the term “parent drug” means any of the presently described compounds herein. Prodrugs can be used to achieve any desired effect, including to enhance properties of the parent drug or to improve the pharmaceutic or pharmacokinetic properties of the parent, including to increase the half-life of the drug in vivo. Prodrug strategies provide choices in modulating the conditions for in vivo generation of the parent drug. Non-limiting examples of prodrug strategies include covalent attachment of removable groups, or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In certain aspects, the prodrug renders the parent compound more lipophilic. In certain aspects, a prodrug can be provided that has several prodrug moieties in a linear, branched, or cyclic manner. For example, non-limiting aspects include the use of a divalent linker moiety such as a dicarboxylic acid, amino acid, diamine, hydroxycarboxylic acid, hydroxyamine, di-hydroxy compound, or other compound that has at least two functional groups that can link the parent compound with another prodrug moiety, and is typically biodegradable in vivo. In some aspects, 2, 3, 4, or 5 prodrug biodegradable moieties are covalently bound in a sequence, branched, or cyclic fashion to the parent compound. Non-limiting examples of prodrugs according to the present disclosure are formed with: a carboxylic acid on the parent drug and a hydroxylated prodrug moiety to form an ester; a carboxylic acid on the parent drug and an amine prodrug to form an amide; an amino on the parent drug and a carboxylic acid prodrug moiety to form an amide; an amino on the parent drug and a sulfonic acid to form a sulfonamide; a sulfonic acid on the parent drug and an amino on the prodrug moiety to form a sulfonamide; a hydroxyl group on the parent drug and a carboxylic acid on the prodrug moiety to form an ester; a hydroxyl on the parent drug and a hydroxylated prodrug moiety to form an ester, a phosphonate on the parent drug and a hydroxylated prodrug moiety to form a phosphonate ester; a phosphoric acid on the parent drug and a hydroxylated prodrug moiety to form a phosphate ester, a hydroxyl on the parent drug and a phosphonate on the prodrug to form a phosphonate ester, a hydroxyl on the parent drug and a phosphoric acid prodrug moiety to form a phosphate ester; a carboxylic acid on the parent drug and a prodrug of the structure HO—(CH₂)₂—O—(C₂₋₂₄ alkyl) to form an ester, a carboxylic acid on the parent drug and a prodrug of the structure HO—(CH₂)₂—S—(C₂₋₂₄ alkyl) to form a thioester, a hydroxyl on the parent drug and a prodrug of the structure HO—(CH₂)₂—O—(C₂₋₂₄ alkyl) to form an ether, a hydroxyl on the parent drug and a prodrug of the structure HO—(CH₂)₂—O—(C₂₋₄ alkyl) to form an thioether, and a carboxylic acid, oxime, hydrazide, hydrazine, amine or hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some aspects, a prodrug is provided by attaching a natural or non-natural amino acid to an appropriate functional moiety on the parent compound, for example, oxygen, nitrogen, or sulfur, and typically oxygen or nitrogen, usually in a manner such that the amino acid is cleaved in vivo to provide the parent drug. The amino acid can be used alone or covalently linked (straight, branched or cyclic) to one or more other prodrug moieties to modify the parent drug to achieve the desired performance, such as increased half-life, lipophilicity, or other drug delivery or pharmacokinetic properties. The amino acid can be any compound with an amino group and a carboxylic acid, which includes an aliphatic amino acid, alkyl amino acid, aromatic amino acid, heteroaliphatic amino acid, heteroalkyl amino acid, heterocyclic amino acid, or heteroaryl amino acid.

### Pharmaceutical Compositions

The compounds described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and a pharmaceutically acceptable carrier or excipient of some sort may be useful in a variety of medical and non-medical applications. For example, pharmaceutical compositions comprising an active compound and an excipient may be useful for the treatment or prevention of a cancer in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer. solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar, detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some aspects, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide. cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide. butylated hydroxyanisol (BHA). butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain aspects, the preservative is an anti-oxidant. In other aspects, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade. chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain aspects, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain aspects, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

### Methods of Treatment

The present disclosure also provides methods for treating or preventing cancer in a subject, comprising administering to the subject a therapeutically effective amount of a compound or composition disclosed herein. The methods can further comprise administering one or more additional therapeutic agents, for example anti-cancer agents or anti-inflammatory agents. Additionally, the method can further comprise administering a therapeutically effective amount of ionizing radiation to the subject.

Methods of killing a cancer or tumor cell are also provided comprising contacting the cancer or tumor cell with an effective amount of a compound or composition as described herein. In some aspects, the compounds can degrade SOS1. The methods can further include administering one or more additional therapeutic agents or administering an effective amount of ionizing radiation.

The disclosed methods can optionally include identifying a patient who is or can be in need of treatment of an oncological disorder. The patient can be a human or other mammal, such as a primate (monkey, chimpanzee, ape, etc.), dog, cat, cow pig, or horse, or other animals having an oncological disorder. In some aspects, the subject can receive the therapeutic compositions prior to, during, or after surgical intervention to remove part or all of a tumor.

The term “neoplasia” or “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue and most invade surrounding tissues, can metastasize to several sites, are likely to recur after attempted removal and may cause the death of the patient unless adequately treated. As used herein, the term neoplasia is used to describe all cancerous disease states and embraces or encompasses the pathological process associated with malignant, hematogenous, ascitic and solid tumors. The cancers which may be treated by the compositions disclosed herein may comprise carcinomas, sarcomas, lymphomas, leukemias, germ cell tumors, or blastomas.

Carcinomas which may be treated by the compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scinious carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, vemucous carcinoma, and carcinoma vilosum.

Representative sarcomas which may be treated by the compositions of the present disclosure include, but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compositions of the present disclosure may be used in the treatment of a lymphoma. Lymphomas which may be treated include mature B cell neoplasms, mature T cell and natural killer (NK) cell neoplasms, precursor lymphoid neoplasms, Hodgkin lymphomas, and immunodeficiency-associated lymphoproliferative disorders.

Representative mature B cell neoplasms include, but are not limited to, B-cell chronic lymphocytic leukemia/small cell lymphoma, B-cell prolymphocytic leukemia, lymphoplasmacytic lymphoma (such as Waldenström macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lyphomatoid granulomatosis, primary mediastinal (thymic) large B-cell lymphoma, intravascular large B-cell lymphoma, ALK+ large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compositions of the present disclosure may be used in the treatment of a Leukemia. Representative examples of leukemias include, but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compositions of the present disclosure may be used in the treatment of a germ cell tumor, for example germinomatous (such as germinoma, dysgerminoma, and seminoma), non germinomatous (such as embryonal carcinoma, endodermal sinus tumor, choriocarcinoma, teratoma, polyembryoma, and gonadoblastoma) and mixed tumors.

The compositions of the present disclosure may be used in the treatment of blastomas, for example hepatoblastoma, medulloblastoma, nephroblastoma, neuroblastoma, pancreatoblastoma, pleuropulmonary blastoma, retinoblastoma, and glioblastoma multiforme.

Representative cancers which may be treated include, but are not limited to: bone and muscle sarcomas such as chondrosarcoma, Ewing's sarcoma, malignant fibrous histiocytoma of bone/osteosarcoma, osteosarcoma, rhabdomyosarcoma, and heart cancer, brain and nervous system cancers such as astrocytoma, brainstem glioma, pilocytic astrocytoma, ependymoma, primitive neuroectodermal tumor, cerebellar astrocytoma, cerebral astrocytoma, glioma, medulloblastoma, neuroblastoma, oligodendroglioma, pineal astrocytoma, pituitary adenoma, and visual pathway and hypothalamic glioma; breast cancers including invasive lobular carcinoma, tubular carcinoma, invasive cribriform carcinoma, medullary carcinoma, male breast cancer, Phyllodes tumor, and inflammatory breast cancer; endocrine system cancers such as adrenocortical carcinoma, islet cell carcinoma, multiple endocrine neoplasia syndrome, parathyroid cancer, phemochromocytoma, thyroid cancer, and Merkel cell carcinoma; eye cancers including uveal melanoma and retinoblastoma; gastrointestinal cancers such as anal cancer, appendix cancer, cholangiocarcinoma, gastrointestinal carcinoid tumors, colon cancer, extrahepatic bile duct cancer, gallbladder cancer, gastric cancer, gastrointestinal stromal tumor, hepatocellular cancer, pancreatic cancer, and rectal cancer; genitourinary and gynecologic cancers such as bladder cancer, cervical cancer, endometrial cancer, extragonadal germ cell tumor, ovarian cancer, ovarian epithelial cancer, ovarian germ cell tumor, penile cancer, renal cell carcinoma, renal pelvis and ureter transitional cell cancer, prostate cancer, testicular cancer, gestational trophoblastic tumor, urethral cancer, uterine sarcoma, vaginal cancer, vulvar cancer, and Wilms tumor, head and neck cancers such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer; hematopoietic cancers such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, hepatosplenic T-cell lymphoma, Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, Mast cell leukemia, mediastinal large B cell lymphoma, multiple myeloma/plasma cell neoplasm, myelodysplastic syndroms, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sezary syndrome, splenic marginal zone lymphoma, and T-cell prolymphocytic leukemia; skin cancers such as basal cell carcinoma, squamous cell carcinoma, skin adnexal tumors (such as sebaceous carcinoma), melanoma, Merkel cell carcinoma, sarcomas of primary cutaneous origin (such as dermatofibrosarcoma protuberans), and lymphomas of primary cutaneous origin (such as mycosis fungoides); thoracic and respiratory cancers such as bronchial adenomas/carcinoids, small cell lung cancer, mesothelioma, non-small cell lung cancer, pleuropulmonary blastoma, laryngeal cancer, and thymoma or thymic carcinoma; HIV/AIDs-related cancers such as Kaposi sarcoma; epithelioid hemangioendothelioma; desmoplastic small round cell tumor, and liposarcoma.

In some aspects, the cancer is a KRAS-mutant cancer. In some aspects, the cancer is KRAS-mutant colorectal cancer.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can also be systemically administered, such as intravenously or orally, optionally in combination with a pharmaceutically acceptable carrier such as an inert diluent, or an assimilable edible carrier for oral delivery. In addition, the active compound can be incorporated into sustained release preparations and/or devices.

For the treatment of oncological disorder, compounds, agents, and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor.

For example, compounds, agents, and compositions disclosed herein can be used in methods of treating cancer wherein the patient is to be treated or is or has been treated with mitotic inhibitors such as taxol or vinblastine, alkylating agents such as cyclophosphamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, imatinid or trastuzumab.

These other substances or radiation treatments can be given at the same time as or at different times from the compounds disclosed herein. Examples of other suitable chemotherapeutic agents include, but are not limited to, altretamine, bleomycin, bortezomib, busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib, gemcitabine, hydroxyurea, idarubicin, ifosfamide, imatinib, irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uraxil, temozolomide, thiotepa, tioguanine/thioguanine, topotexan, treosulfan, vinblastine, vincristine, vindesine, and vinorelbine. Examples of suitable immunotherapeutic agents include, but are not limited to, alemtuzumab, cetuximab, gemtuzumab, iodine 131 tositumomab, rituximab, and trastuzumab. Cytotoxic agents include, for example, radioactive isotopes and toxins of bacterial, fungal, plant, or animal origin. Also disclosed are methods of treating an oncological disorder comprising administering an effective amount of a compound described herein prior to, subsequent to, and/or in combination with administration of a chemotherapeutic agent, an immunotherapeutic agent, a radiotherapeutic agent, or radiotherapy.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some aspects, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol.

In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art.

The dosage can be adjusted by the individual physician in the event of any counterindications. Dosage can vary and can be administered in one or more dose administrations daily, for one or several days.

### Kits

Kits for practicing the methods described herein are further provided. By “kit” is intended any manufacture (e.g., a package or a container) comprising at least one reagent, e.g., any one of the compounds described herein. The kit can be promoted, distributed, or sold as a unit for performing the methods described herein. Additionally, the kits can contain a package insert describing the kit and methods for its use. Any or all of the kit reagents can be provided within containers that protect them from the external environment, such as in sealed containers or pouches.

To provide for the administration of such dosages for the desired therapeutic treatment, in some aspect s, pharmaceutical compositions disclosed herein can comprise between 0.1% and 45%, and especially, 1 and 15%, by weight of the total of one or more of the compounds based on the weight of the total composition including carriers and/or diluents. Illustratively, dosage levels of the administered active ingredients can be: intravenous 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasally, 0.01 to about 20 mg/kg; and aerosol, 0.01 to about 20 mg/kg of animal (body) weight.

Also disclosed are kits that comprise a composition comprising a compound disclosed herein in one or more containers. The disclosed kits can optionally include pharmaceutically acceptable carriers and/or diluents. In one aspect, a kit includes one or more other components, adjuncts, or adjuvants as described herein. In another aspect, a kit includes one or more anti-cancer agents, such as those agents described herein. In one aspect, a kit includes instructions or packaging materials that describe how to administer a compound or composition of the kit. Containers of the kit can be of any suitable material, e.g., glass, plastic, metal, etc., and of any suitable size, shape, or configuration. In one aspect, a compound and/or agent disclosed herein is provided in the kit as a solid, such as a tablet, pill, or powder form. In another aspect, a compound and/or agent disclosed herein is provided in the kit as a liquid or solution. In one aspect, the kit comprises an ampoule or syringe containing a compound and/or agent disclosed herein in liquid or solution form.

The following particular aspects of the present disclosure are also provided:

Aspect 1. A compound of Formula I or Formula II

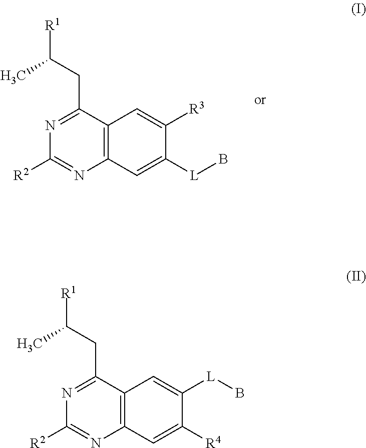

or a pharmaceutically acceptable salt or derivative thereof,
wherein:
R¹ is aryl optionally substituted with one or more groups independently selected from halo, amino, C₁-C₆ alkyl, and C₁-C₆ haloalkyl;
R² is selected from H or C₁-C₆ alkyl;
R³ and R⁴ are each C₁-C₆ alkoxy;
L is a linker, and
B is an E3 ubiquitin ligase-recruiting moiety.

Aspect 2. The compound of aspect 1, wherein R¹ is unsubstituted phenyl.

Aspect 3. The compound of aspect 1, wherein R¹ is phenyl substituted with C₁-C₆ haloalkyl.

Aspect 4. The compound of aspect 3, wherein R¹ is phenyl substituted with trifluoromethyl.

Aspect 5. The compound of aspect 1, wherein R¹ is phenyl substituted with amino and C₁-C₆ haloalkyl.

Aspect 6. The compound of aspect 5, wherein R¹ is phenyl substituted with amino and trifluoromethyl.

Aspect 7. The compound of aspect 1, wherein R¹ is selected from:

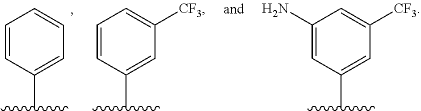

Aspect 8. The compound of any one of aspects 1-7, wherein R² is H.

Aspect 9. The compound of any one of aspects 1-7, wherein R² is C₁-C₆ alkyl.

Aspect 10. The compound of aspect 9, wherein R² is methyl.

Aspect 11. The compound of any one of aspects 1-10, R³ is methoxy.

Aspect 12. The compound of any one of aspects 1-10, R⁴ is methoxy.

Aspect 13. The compound of any one of aspects 1-12, wherein the linker is selected from the group consisting of a moiety of Formula L1, Formula L2, Formula L3, Formula L4, Formula L5, Formula L6, Formula L7, Formula L8, Formula L9, or Formula L10:

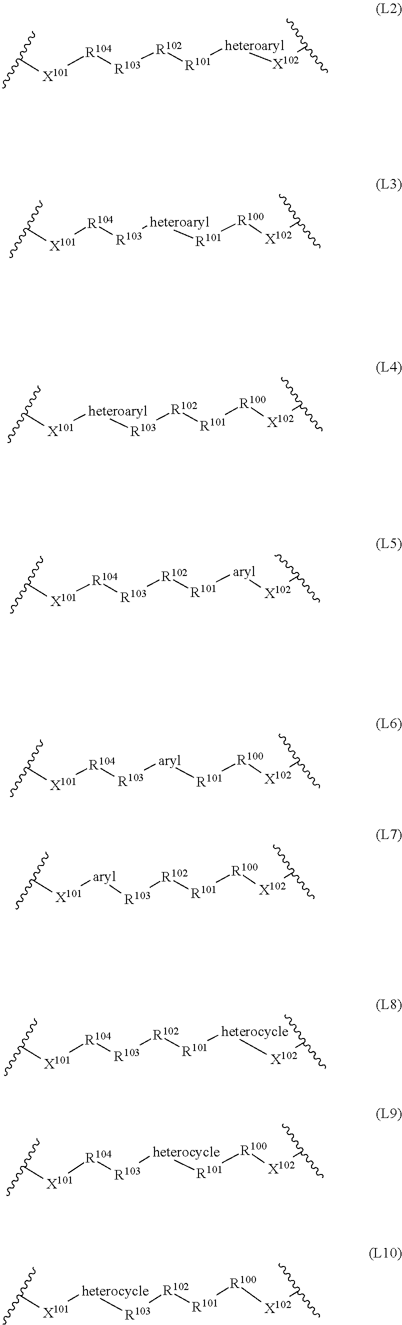

wherein:
X¹⁰¹ and X¹⁰² are independently at each occurrence selected from a bond, aryl, heteroaryl, cycloalkyl, heterocycle, NR¹³⁰, C(R¹³⁰)₂, O, C(O), and S;
R¹⁰⁰, R¹⁰¹, R¹⁰², R¹⁰³, and R¹⁰⁴ are independently at each occurrence selected from the group consisting of a bond, alkyl, —C(O)—, —C(O)O—, —OC(O)—, —SO₂—, —S(O)—, C(S)—, —C(O)NR¹³⁰—, —NR¹³⁰C(O)—, —O—, —S—, —NR¹³⁰—, —C(R¹³⁰R¹³⁰)—, —P(O)(OR¹⁰⁶))—, —R(O)(OR¹⁰⁶)—, alkenyl, alkynyl, haloalkyl, alkoxy, aryl, heterocycloalkyl, cycloalkyl, heteroaryl, lactic acid, or glycolic acid, each of which may be optionally substituted with one or more (for example, 1, 2, 3, or 4) substituents independently selected from R¹⁴⁰;
R¹⁰⁶ is independently at each occurrence selected from the group consisting of hydrogen, alkyl, arylalkyl, heteroarylalkyl, alkenyl, alkynyl, aryl, heteroaryl, cycloalkyl, or heterocycloalkyl;
R¹³⁰ is independently as each occurrence selected from the group consisting of hydrogen, alkyl, cycloalkyl, heterocycloalkyl, aryl, heteroaryl, —C(O)H, —C(O)OH, —C(O)alkyl, —C(O)Oalkyl, —C(O)(cycloalkyl, heterocycloalkyl, aryl, or heteroaryl), —C(O)O(cycloalkyl, heterocycloalkyl, aryl, or heteroaryl), alkenyl, or alkynyl; and
R¹⁴⁰ is independently at each occurrence selected from the group consisting of hydrogen, alkyl, alkenyl, alkynyl, fluoro, bromo, chloro, hydroxyl, alkoxy, azide, amino cyano, —NH(alkyl, cycloalkyl, heterocyloalkyl, aryl, or heteroaryl), —N(independently alkyl, cycloalkyl, heterocycloalkyl, aryl, or heteroaryl), —NHSO₂(alkyl, cycloalkyl, heterocycloalkyl, aryl, or heteroaryl), —N(alkyl, cycloalkyl, heterocycloalkyl, aryl, or heteroaryl)SO₂alkyl, —NHSO₂alkenyl, —N(alkyl)SO₂alkenyl, —NHSO₂alkynyl, —N(alkyl)SO₂alkynyl, haloalkyl, cycloalkyl, heterocycloalkyl, aryl, or heteroaryl.

Aspect 14. The compound of any one of aspects 1-13, wherein B is selected from

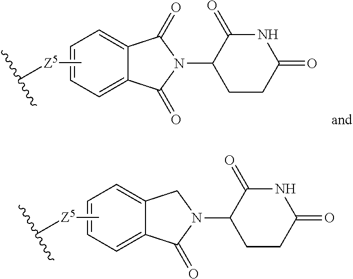

wherein Z⁵ is selected from O, N(R^{a}), and CH₂.

Aspect 15. The compound of any one of aspects 1-13, wherein B is selected from

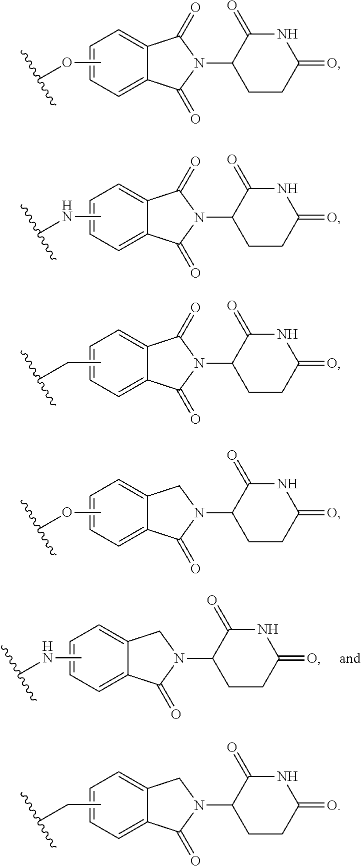

Aspect 16. The compound of any one of aspects 1-13, wherein B is selected from

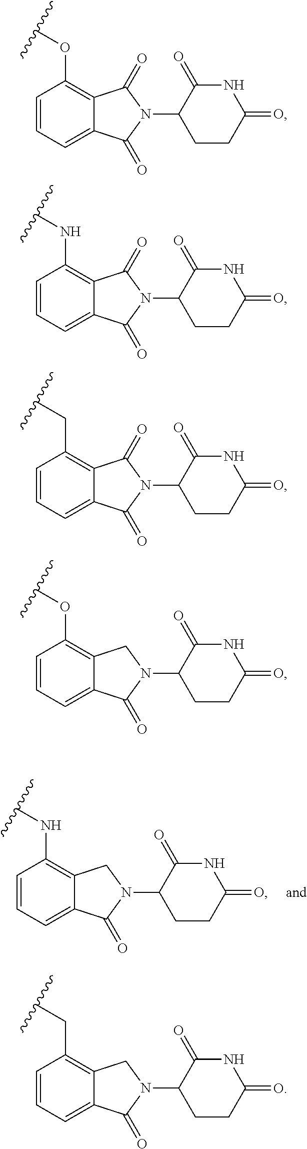

Aspect 17. The compound of an one of aspects 1-13, wherein Bis selected from

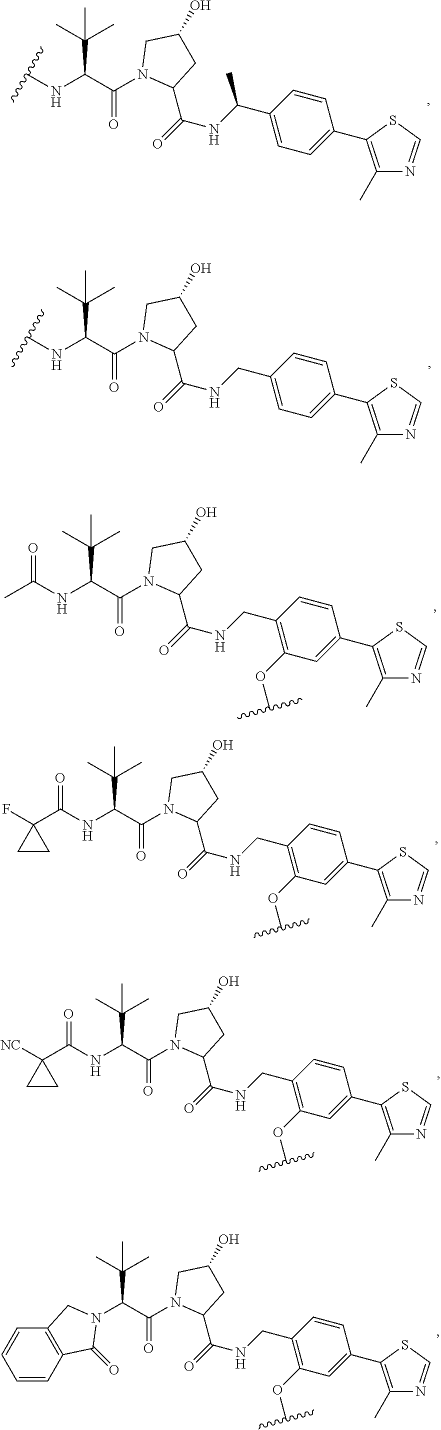 [SMILES: *N[C@H](C(=O)N1C[C@H](O)CC1C(=O)N[C@@H](C)c1ccc(-c2scnc2C)cc1)C(C)(C)C ; *N[C@H](C(=O)N1C[C@H](O)CC1C(=O)NCc1ccc(-c2scnc2C)cc1)C(C)(C)C ; *Oc1cc(-c2scnc2C)ccc1CNC(=O)C1C[C@@H](O)CN1C(=O)[C@@H](NC(C)=O)C(C)(C)C ; *Oc1cc(-c2scnc2C)ccc1CNC(=O)C1C[C@@H](O)CN1C(=O)[C@@H](NC(=O)C1(F)CC1)C(C)(C)C ; *Oc1cc(-c2scnc2C)ccc1CNC(=O)C1C[C@@H](O)CN1C(=O)[C@@H](NC(=O)C1(C#N)CC1)C(C)(C)C ; *Oc1cc(-c2scnc2C)ccc1CNC(=O)C1C[C@@H](O)CN1C(=O)[C@@H](N1Cc2ccccc2C1=O)C(C)(C)C]

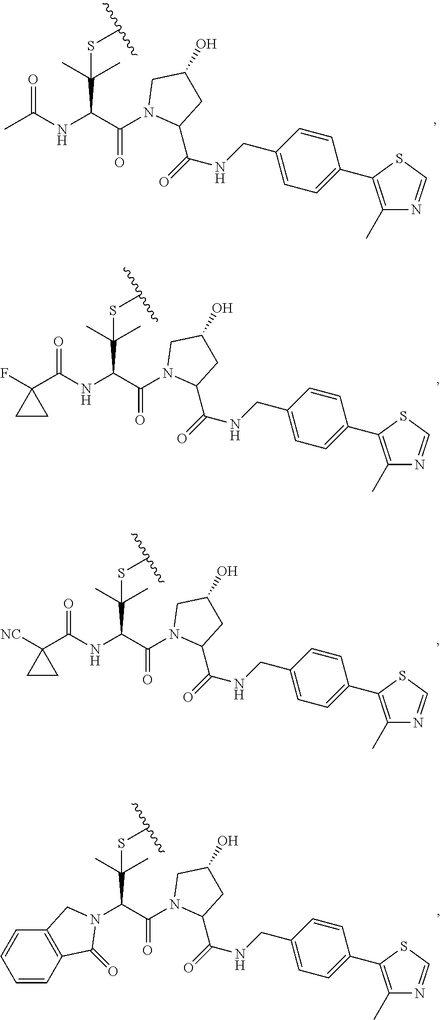 [SMILES: *SC(C)(C)[C@H](NC(C)=O)C(=O)N1C[C@H](O)CC1C(=O)NCc1ccc(-c2scnc2C)cc1 ; *SC(C)(C)[C@H](NC(=O)C1(F)CC1)C(=O)N1C[C@H](O)CC1C(=O)NCc1ccc(-c2scnc2C)cc1 ; *SC(C)(C)[C@H](NC(=O)C1(C#N)CC1)C(=O)N1C[C@H](O)CC1C(=O)NCc1ccc(-c2scnc2C)cc1 ; *SC(C)(C)[C@@H](C(=O)N1C[C@H](O)CC1C(=O)NCc1ccc(-c2scnc2C)cc1)N1Cc2ccccc2C1=O]

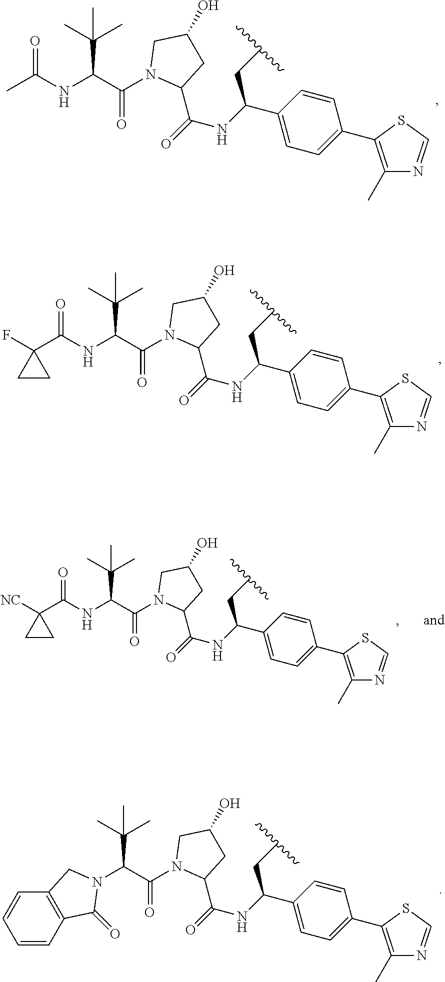 [SMILES: *C[C@H](NC(=O)C1C[C@@H](O)CN1C(=O)[C@@H](NC(C)=O)C(C)(C)C)c1ccc(-c2scnc2C)cc1 ; *C[C@H](NC(=O)C1C[C@@H](O)CN1C(=O)[C@@H](NC(=O)C1(F)CC1)C(C)(C)C)c1ccc(-c2scnc2C)cc1 ; *C[C@H](NC(=O)C1C[C@@H](O)CN1C(=O)[C@@H](NC(=O)C1(C#N)CC1)C(C)(C)C)c1ccc(-c2scnc2C)cc1 ; *C[C@H](NC(=O)C1C[C@@H](O)CN1C(=O)[C@@H](N1Cc2ccccc2C1=O)C(C)(C)C)c1ccc(-c2scnc2C)cc1]

Aspect 18. A pharmaceutical composition comprising a compound of any one of aspects 1-17, or a pharmaceutically acceptable salt or derivative thereof, and a pharmaceutically acceptable carrier or excipient.

Aspect 19. A method of treating cancer in a subject in need thereof comprising administering to the subject a therapeutically effective amount of a compound of any one of aspects 1-17, or a pharmaceutically acceptable salt or derivative thereof, or a pharmaceutical composition of aspect 18.

Aspect 20. The method of aspect 19, wherein the cancer is a KRAS-mutant cancer.

Aspect 21. The method of aspect 19 or 20, wherein the cancer is KRAS-mutant colorectal cancer.

Aspect 22. The method of any one of aspects 19-21, wherein the subject is a human.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are put forth so as to provide those of ordinary skill in the art with a complete disclosure and description of how the compounds, compositions, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

Development of new SOS1 degraders to target KRAS-mutant colorectal cancer KRAS driver mutations are prevalent in colorectal cancer (CRC), associated with poor prognosis and resistance to therapy. Thus, representing a suitable target for CRC, but its direct blockade has been challenging. Inhibition of SOS1, a guanine nucleotide exchange factor, playing a key role in transducing mutant KRAS downstream signaling, has arisen as an attractive approach to target KRAS-mutant CRC. Here, we describe the development of novel SOS1 degraders and evaluation of their activity in patient-derived CRC models. The design of SOS1 degraders in the form of proteolysis-targeting chimera was based on the crystal structures of E3 ligase cereblon and SOS1. The synthesis used the 6- and 7-OH groups of a quinazoline core as anchor points to connect lenalidomide with a linker. Fifteen candidate compounds were synthesized and screened for SOS1 degradation. P7 was found to have up to 92% SOS1 degradation. SOS1 degradation induced by P7 was demonstrated in both CRC cell lines and patient-derived organoid (PDO) models with excellent specificity as shown in global proteomics analysis. SOS1 degrader P7 demonstrated superior activity in inhibiting growth of CRC PDOs with IC₅₀ 5 times lower than that of SOS1 inhibitor BI3406. In summary, we developed new SOS1 degraders and demonstrated SOS1 degradation as a feasible and novel therapeutic strategy for KRAS-mutant CRC.

Here, we described the design and synthesis of SOS1 inhibitor-based degraders utilizing cereblon as the E3 ligase. Our SOS1 degrader achieved up to 92% SOS1 degradation in both CRC cell lines and PDOs with excellent specificity for SOS1. Our SOS1 degrader demonstrated superior activity to SOS1 inhibitor BI3406 in inhibiting growth of CRC PDOs. We demonstrated SOS1 degradation as a feasible and novel therapeutic strategy for targeting SOS1 in KRAS-mutant CRC.

### Results and Discussion

### Design of SOS1 Degrader

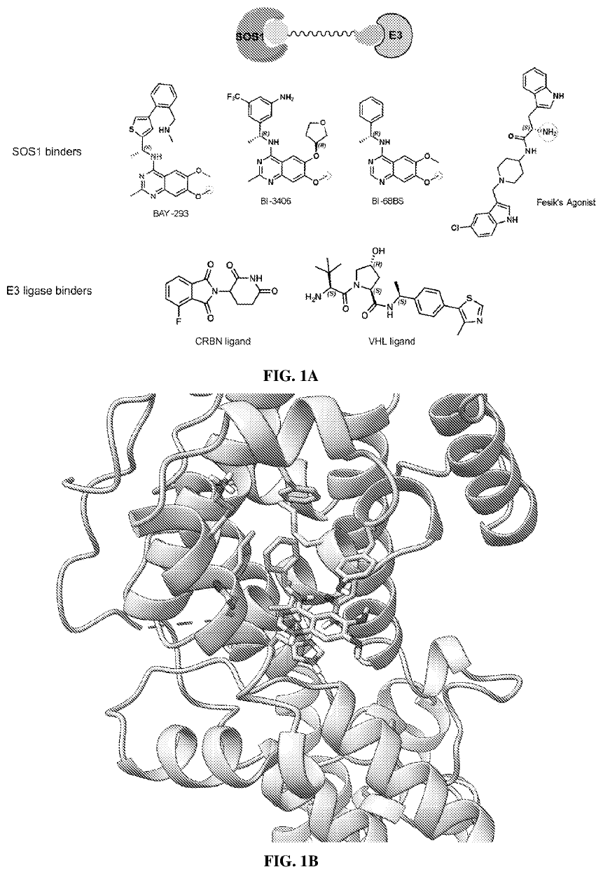

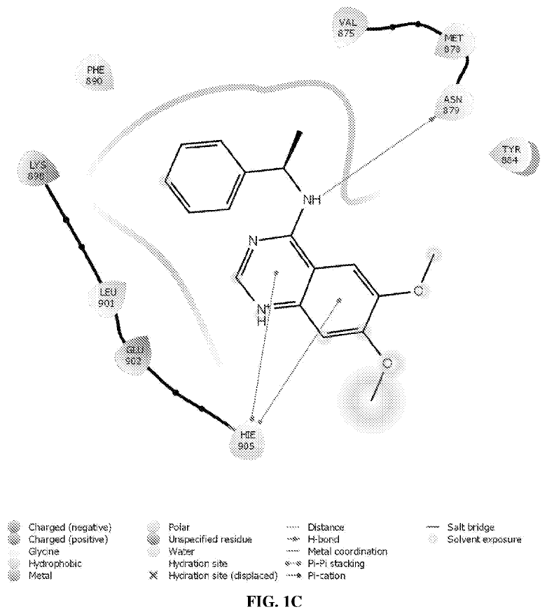

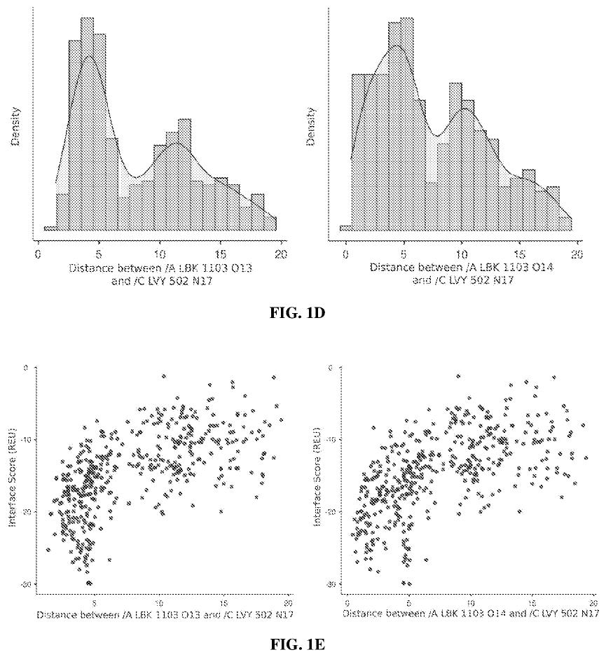

Recent advances in the discovery of small molecule SOS1 binders including both SOS1 inhibitors and agonists allowed us to design and synthesize heterobifunctional SOS1 proteolysis targeting chimera (PROTAC) degraders with diverse molecular structures. Our design of SOS1 degrader was inspired by the structure-activity relationship of BAY293 with IC₅₀ measured using the KRAS^{G12C}-SOS1^{cat} interaction and EGFR inhibition assays,¹⁵ which provided the information of essential components in the molecular structure that are required to retain high binding affinity to SOS1 (FIG. 1A). Our synthesis of SOS1 degraders that are potent and selective was informed by the crystal structure of SOS1 (PDB: 6SFR) in a complex with SOS1 binder BI68BS (FIG. 1B).¹⁷ BI68BS is an analog of SOS1 inhibitor BI3406 with 9.7 nM binding affinity to SOS1 in preclinical studies¹⁷ and of SOS1 inhibitor BI1701963, currently in a phase I trial of patients with KRAS-mutant advanced solid tumors (NCT04111458).²⁴ Visual inspection and structural interaction fingerprint analysis of BI68BS with SOS1^{25,26} demonstrated that in addition to the hydrophobic pocket and H-bond interaction with Asn879, 1) both the 6- and 7-methoxy groups are exposed to solvent; 2) π-π stacking between the quinazoline core of BI68BS and His905 is essential for its activity (FIG. 1C). This interaction can also be fulfilled by phthalazine and 2, 4, 6-triaza-2-indanyl groups in other reported SOS1 inhibitors.^{6,27}

We then performed protein-protein docking of BI68BS-bound SOS1 with lenalidomide-bound cereblon to determine the optimal length and conformation of the linkers of SOS1 PROTAC degraders. As reported in previous studies, in silico modeling of the interactions between proteins of interest and E3 ligases, especially cereblon could be very informative for the favored conformations of ternary complexes, which are putative intermediates of targeted protein degradation induced by PROTAC.²⁸ A protein-protein docking experiments provided 500 ranked conformation models of the BI68BS-bound SOS1 (PDB: 6SFR) and the lenalidomide-bound cereblon (PDB: 4TZ4). Among the models having the distance between BI68BS (6- or 7-methoxy group) and lenalidomide (amino group) within 20 Å and total energy less than −800 Rosetta energy units (REU), we found that the distances between lenalidomide amino group and BI68BS methoxy groups had bimodal distribution with a median of 6.2 Å for BI68BS 6-methoxy group and 6.1 Å for BI68BS 7-methoxy group (FIG. 1D). Interface scores, which represent the energy of the interactions across the protein-protein interface, were also estimated from the docking experiments. The interface scores were lowest when plotted against the distances between BI68BS methoxy groups and the lenalidomide amino group, with favorable conformations with such distances at approximately 4-5 Å. (FIG. 1E). In an example of the most favorable models (FIG. 1F), BI681BS, as expected, can have the 6- and 7-methoxy groups serve as the sites for linker attachment. For lenalidomide, although the oxo-isoindolinyl sp2 carbons can serve as potential linker anchor points, the 4-amino group can certainly be utilized as a nucleophile for linker attachment.³⁰ The distances between the potential linker attachment sites of BI68BS and lenalidomide could be as short as 4-5 Å, which prompted us to focus our initial syntheses of SOS1 degraders on those with relatively short linkers. In summary, we designed our SOS1 degraders based on structural analysis and computational modeling. This approach, yet only proven to be partially accurate in guiding the discovery of effective PROTAC degraders in the literature, turned out to be very helpful in guiding our initial focus on SOS1 degrader synthesis and subsequently identified a compound with short linker for effective SOS11 degradation.

### Chemical Synthesis of SOS1 Degrader

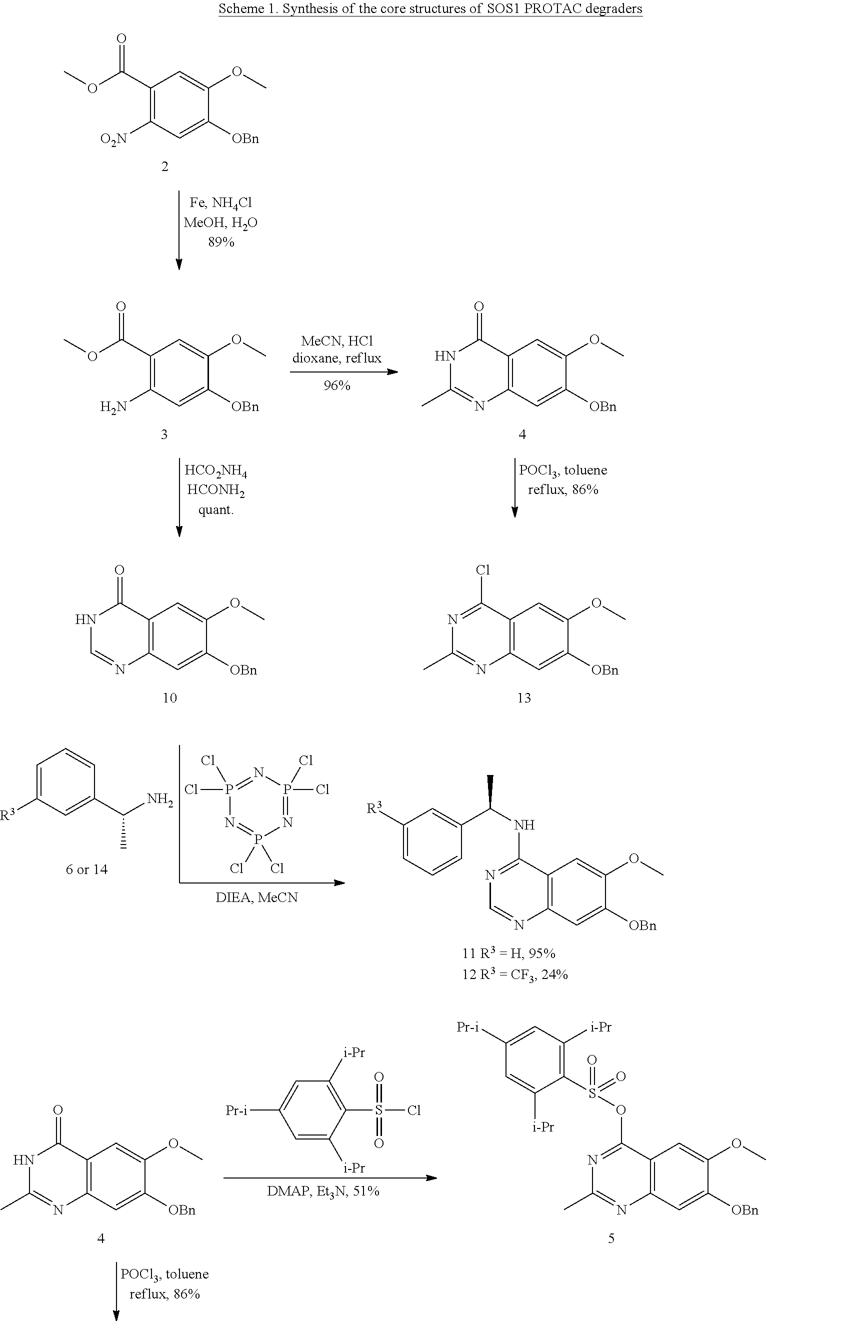

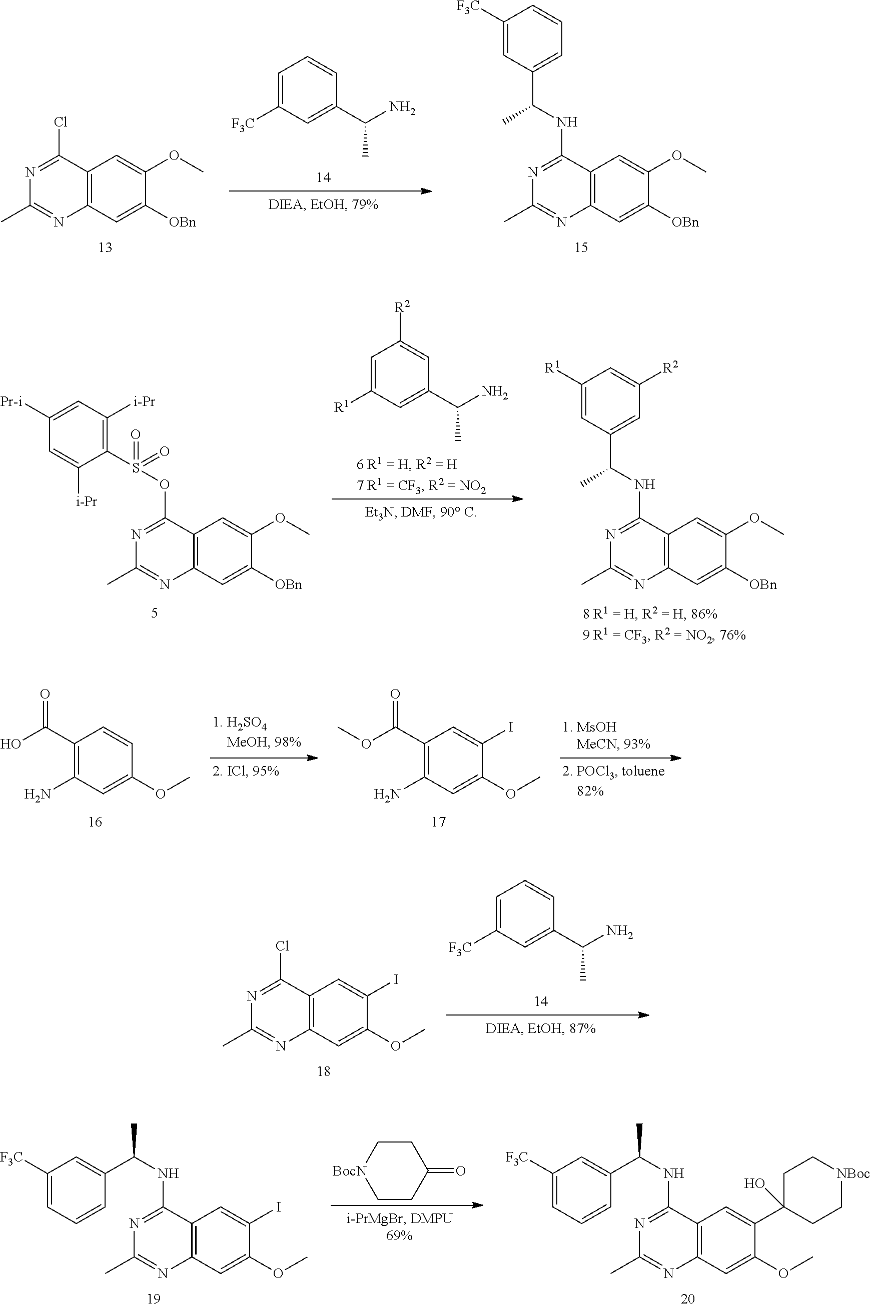

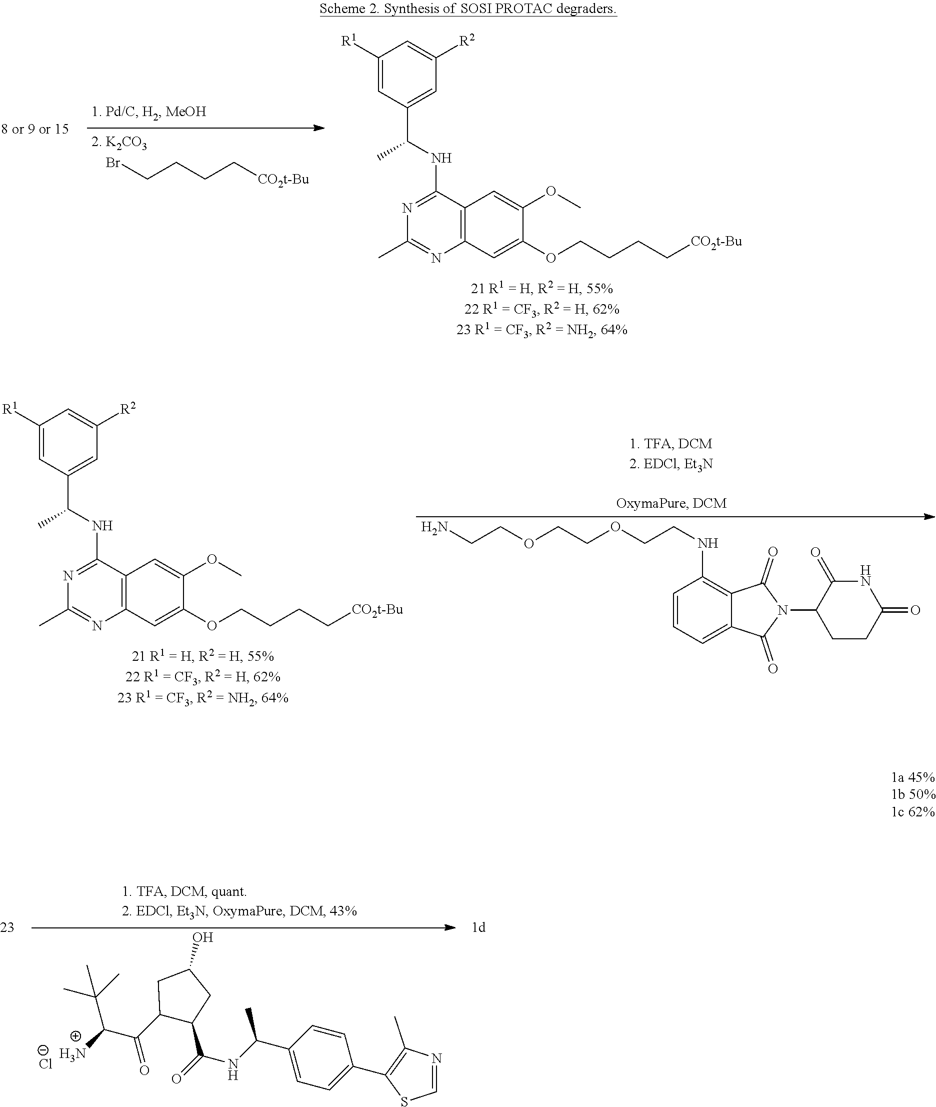

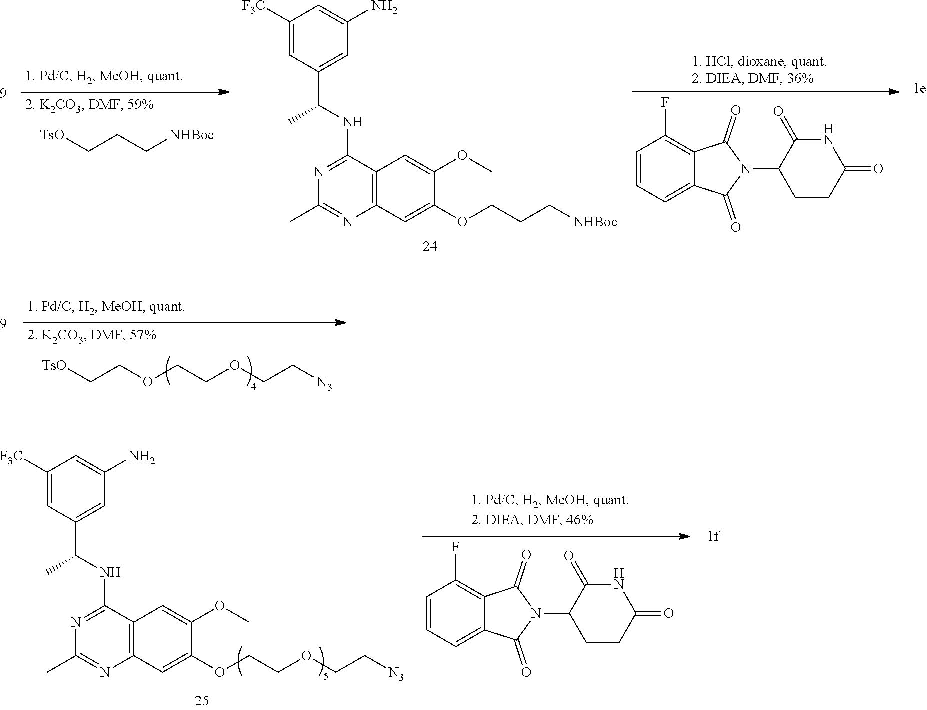

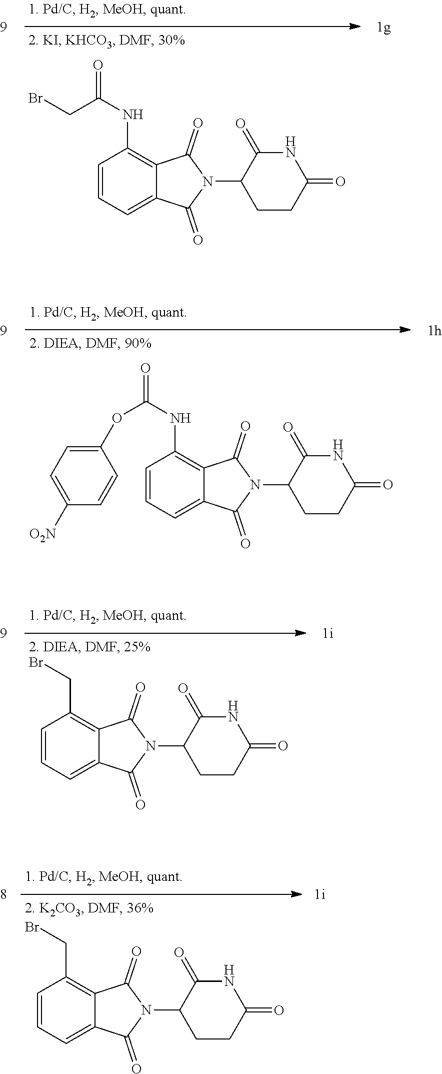 [SMILES: O=C1CCC(N2C(=O)c3cccc(NC(=O)CBr)c3C2=O)C(=O)N1 ; O=C1CCC(N2C(=O)c3cccc(NC(=O)Oc4ccc([N+](=O)[O-])cc4)c3C2=O)C(=O)N1 ; O=C1CCC(N2C(=O)c3cccc(CBr)c3C2=O)C(=O)N1 ; O=C1CCC(N2C(=O)c3cccc(CBr)c3C2=O)C(=O)N1]

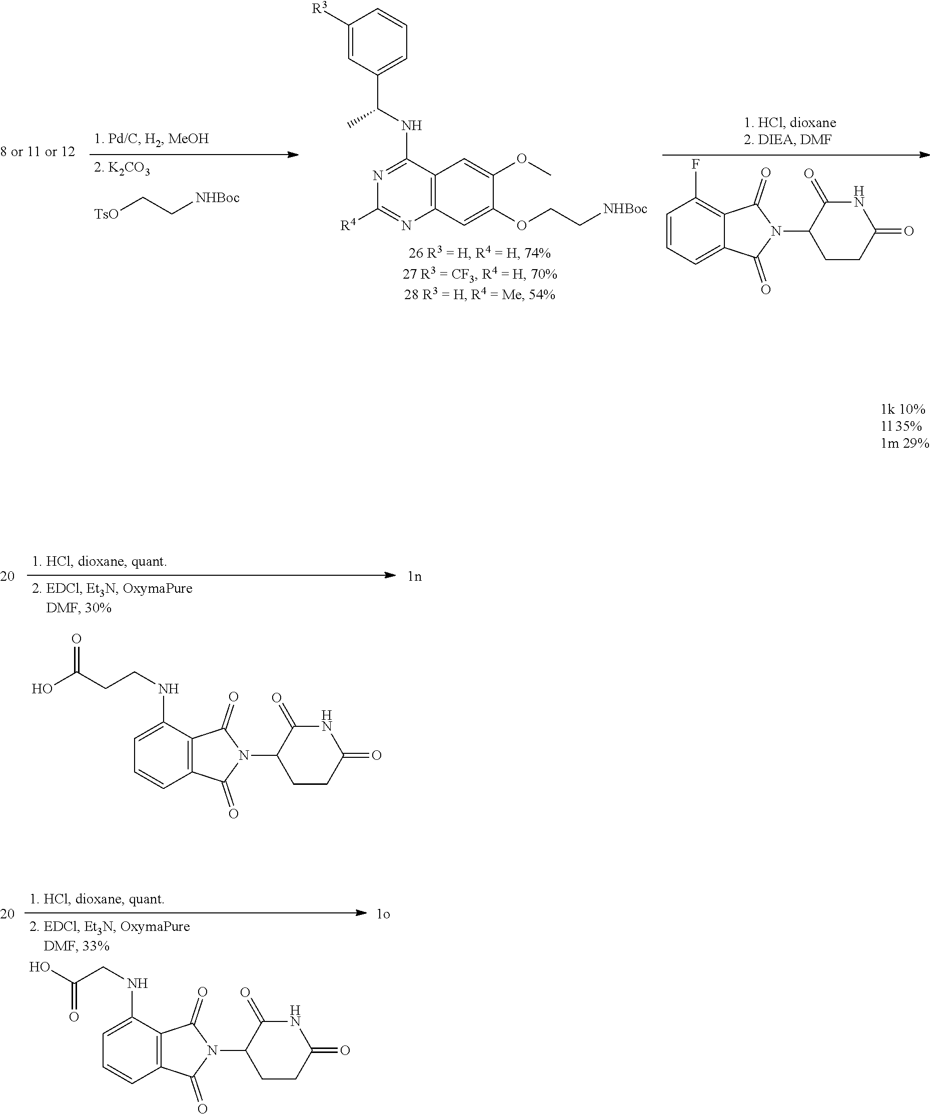

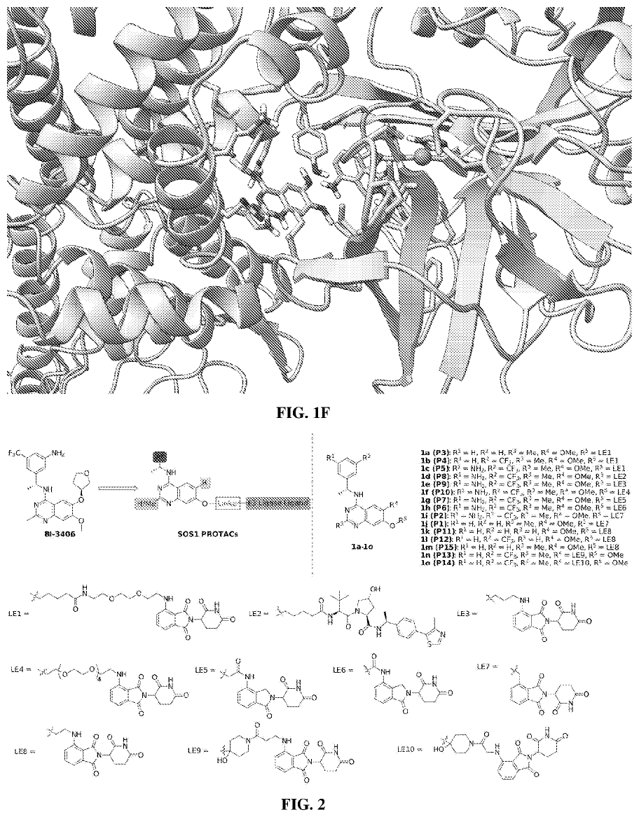

The design of SOS1 PROTAC degrader was based on SOS1:KRAS interaction inhibitor BI3406 and cereblon binders, which is different from a recently reported agonist-based SOS1 PROTAC degrader utilizing VHL E3 ligase.³¹ Variations on the core structure in combination with different linkers and E3 ligase ligands led to 15 different SOS1 degraders (FIG. 2). The synthesis of core structures is depicted in Scheme 1. Starting from commercially available methyl benzoate 2, iron mediated nitro group reduction afforded aniline 3 in 89% yield, which is subjected to acid catalyzed condensation with acetonitrile to create quinazolinone 4 in good yield. Tautomerization and sulfonate formation provided intermediate 5 and the subsequent SNAr reaction by addition of amines gave chiral 4-aminoquinazoline 8 and 9 in 86% and 76% yields, respectively. Hydrogenolysis released the free hydroxyl group, which was able to attach proper PROTAC linkers and E3 ligase ligands. Alternatively, quinazolinone 4 can be converted to chloride 13, which underwent SNAr to generate 15 for further modifications. To compare 2-methyl and 2-unsubstituted quinazolines on the degradation efficiency of SOS1 degraders, quinazolinone 10 was prepared by condensation of 3 with formamide in the presence of ammonium formate. One-pot S_{N}Ar was accomplished under the effect of phosphonitrilic chloride trimer. Core structure 11 and 12 were obtained in good to excellent yields. To evaluate the effect of linker at a different attachment point, compound 20 was prepared in analogous fashion. Commenced from 16 via sequential methylation, iodination, condensation, and chlorination, 18 was generated in overall good yield, which, upon SNAr substitution, proceeded to halogen-metal exchange and addition to carbonyl group to afford core structure 20.

With core compounds 8, 9, 11, 12, 15 and 20 in hands, the SOS1 degrader synthesis was performed according to Scheme 2 in a modular approach. Starting from compounds 8, 9 or 15, hydrogenation of the benzyl group followed by alkylation of tert-butyl bromocarboxylate furnished compounds 21-23, which were ready to couple E3 ligase ligands with an amino tether after acid treatment. Under the effects of carbodiimide and Oxymapure, SOS1 PROTACs 1a-1d were prepared in decent yields. Non-cleavable linkers such as 3-aminopropanol and 17-amino-3, 6, 9, 12, 15-pentaoxaheptadecan-1-ol were also used to create SOS1 PROTACs. As illustrated in Scheme 2, after hydrogenation of the benzyl group and alkylation of the corresponding linker tosylate with amino group protected with Boc, intermediates 24 and 25 were obtained in good yields, which were derivatized to 1e and 1f respectively upon successful SNAr reactions after acid treatment. SOS1 PROTACs 1g-1j and 1m bearing shorter linkers were synthesized by reacting compounds 9 or 28 with proper thalidomide or lenalidomide derivatives. Similarly, 1k and 1l were generated from the 2-unsubstituted 4-aminoquinazolines. The last two SOS1 PROTACs, 1n and 1o were prepared from 20 by reacting with thalidomide substituted carboxylic acids under standard amide coupling conditions. In summary, we adopted synthetic routes that can be easily adapted to the synthesis of multiple SOS1 PROTAC degrader candidates. The flexibility in our synthetic approach allowed fast access to SOS1 degraders with diverse linkerology which enables study of structural-activity relationships for SOS1 degradation.

### Targeted SOS1 Degradation in CRC

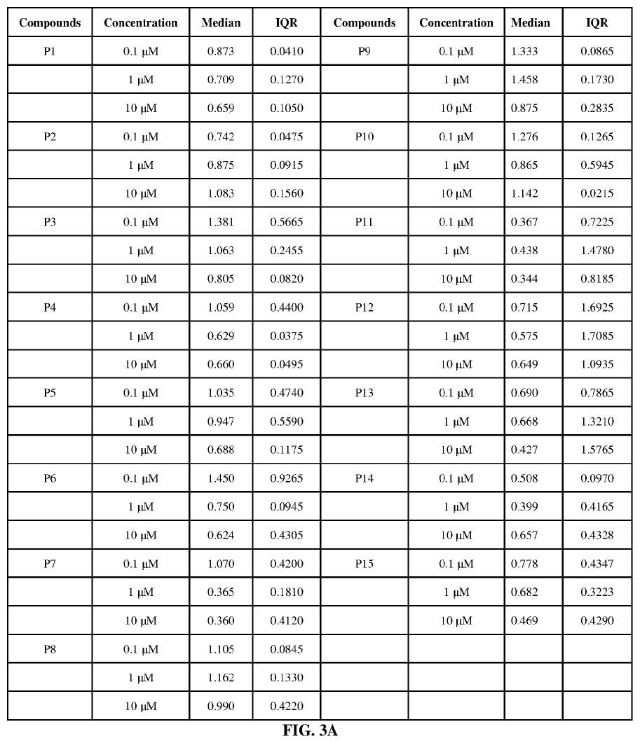

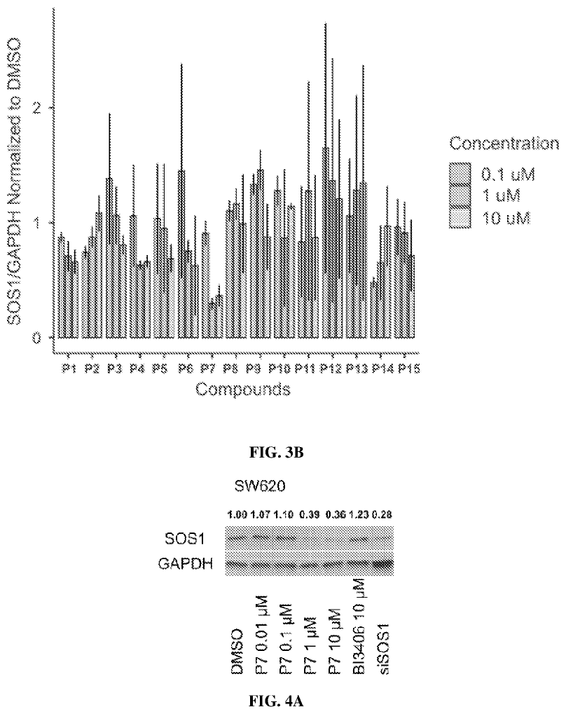

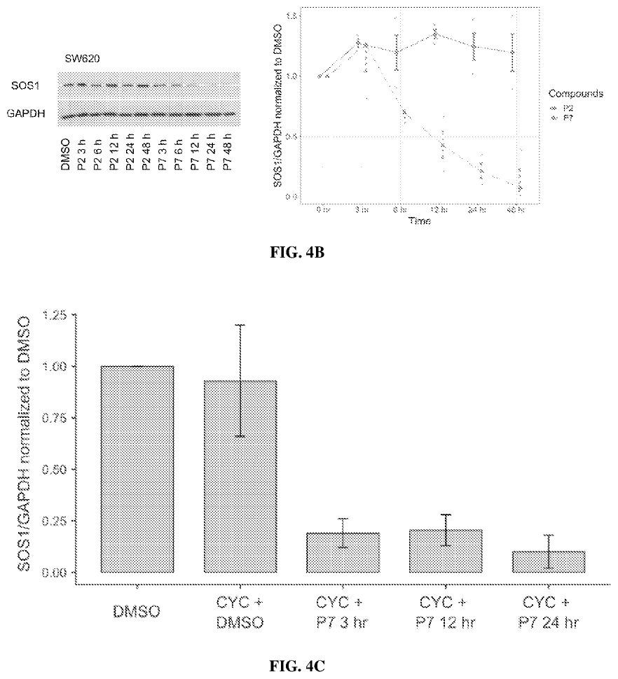

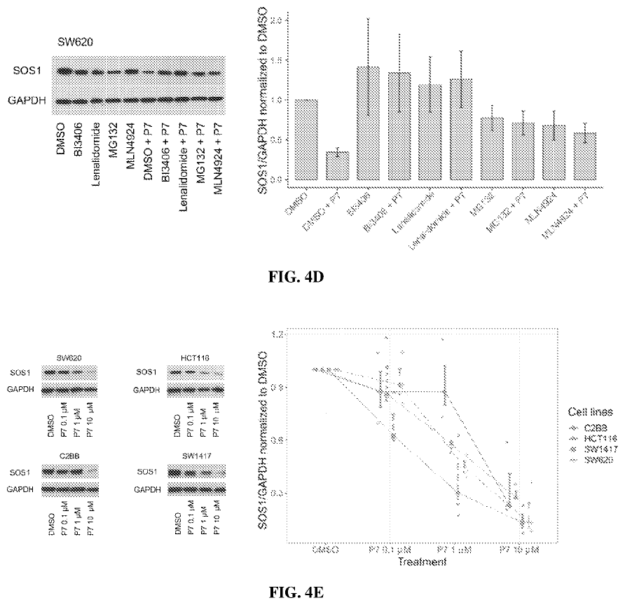

We screened the synthesized SOS1 degrader candidates for SOS1 degradation by immunoblots with a validated and selective SOS1 antibody. Treatment of CRC cell line SW620 for 6 hours with SOS1 degrader candidates showed that P7 among others at 1 μM led to a 64% degradation of SOS1. The degree of SOS1 degradation by various SOS1 degrader candidates are summarized in FIGS. 3A and 3B. P7 induced SOS1 degradation was compared to SOS1 inhibitor BI3406 and siRNA of SOS1, where BI3406 as expected did not induce SOS1 for degradation. The level of SOS1 degradation by P7 was comparable with that by genetic knockdown (FIG. 4A). SOS1 degradation by P7 at lpM was time-dependent. In contrast to some of the other protein PROTAC degraders,³⁰ significant SOS1 degradation was observed at 24 hours or longer with 92% degradation at 48 hours. Such time-dependent SOS1 degradation was not observed in inactive compound P2 (FIG. 4B). To investigate the effect of protein resynthesis on the relatively delayed SOS1 degradation by P7, we pretreated SW620 cells with protein synthesis inhibitor cycloheximide prior to the addition of P7, more than 75% SOS1 degradation was achieved starting at 3 hours and sustained for longer time period (FIG. 4C). This observation suggested that SOS1 protein resynthesis may be rapid upon targeted degradation, which should be further investigated in the future preclinical and clinical applications of SOS1 degraders. The mode of action of P7 for targeted SOS1 degradation was confirmed by the lack of significant SOS1 degradation by P7 after pretreatment with excessive amount of BI3406, lenalidomide, neddylation inhibitor MLN4924, and proteasome inhibitor MG132 (FIG. 4D). P7-induced SOS1 degradation was indeed mediated by the engagement of SOS1 and the ubiquitin-proteasome system. In addition to SW620, P7 also induced SOS1 degradation in other CRC cell lines HCT116, C2BB, and SW1417 in a concentration-dependent manner (FIG. 4E).

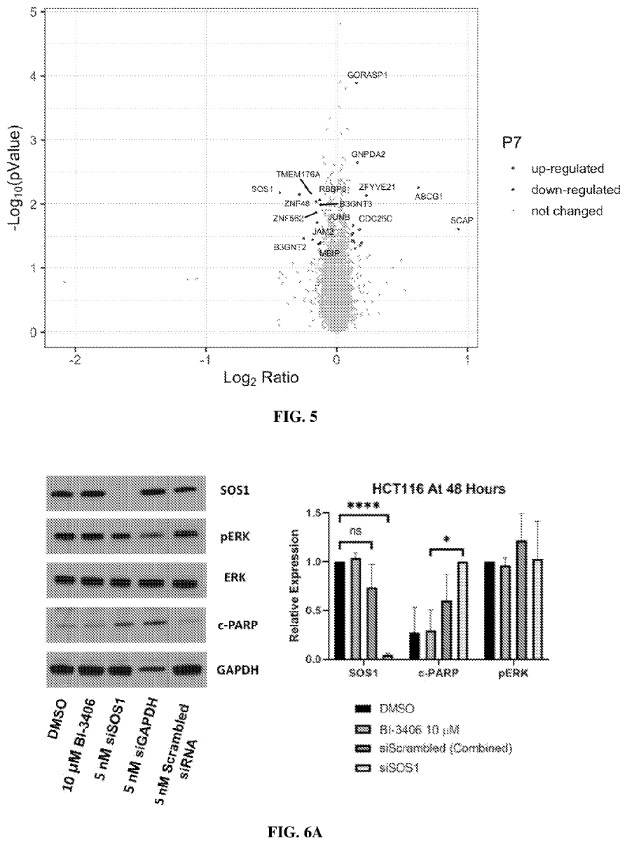

We then assessed the specificity of SOS1 degradation by 1 μM P7 at 24 hours by global proteomics analysis in SW620 cells. As shown in FIG. 5, SOS1 was among the most degraded proteins without cereblon-binding lenalidomide induced degradation of GSPT1³² and the zinc finger transcription factors such as IKZF1/3.³³ We also found that several proteins are significantly upregulated after 24-hour treatment with P7. Both ABCG1 and SCAP were reported to be involved in cellular cholesterol homeostasis,^{34,35} which may be important in the survival of cancer cells with upregulated EGFR-RAS signaling.³⁴ Although it has been reported that MEK1/2 inhibitors promoted protein degradation of ABCG1 in CHO and HuH7 cells,³⁶ upregulation of ABCG1 upon P7-induced SOS1 degradation may serve as a potential resistance mechanism and target in CRC cells. Lack of known direct interaction between SOS1 and ABCG1 or SCAP suggested that their upregulation is a result of altered signaling changes and transcriptional effects due to targeted SOS1 degradation. Studies such as transcriptomic and phosphoproteomics studies could be proposed to evaluate these cellular adaptation mechanisms in the setting of acute SOS1 degradation. In summary, we identified P7 as the most effective compound for SOS1 degradation in multiple CRC cell lines. The effect of P7 was mediated by the ubiquitin-proteasome system with its degradation kinetics affected by rapid SOS1 resynthesis. Our proteomics analysis not only showed that P7 is highly specific for SOS1 degradation, but also revealed upregulation of proteins that may contribute to cellular adaptation to SOS1 degradation.

### Effect of SOS1 Degradation in CRC

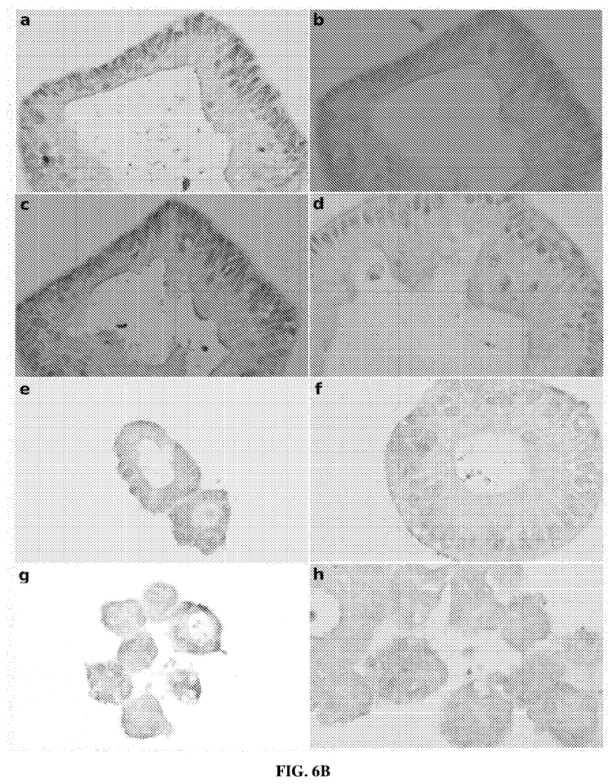

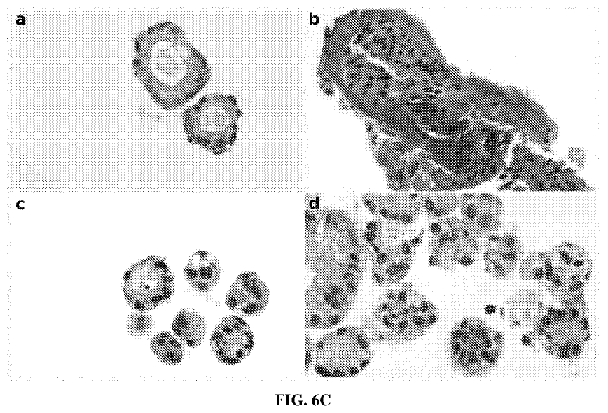

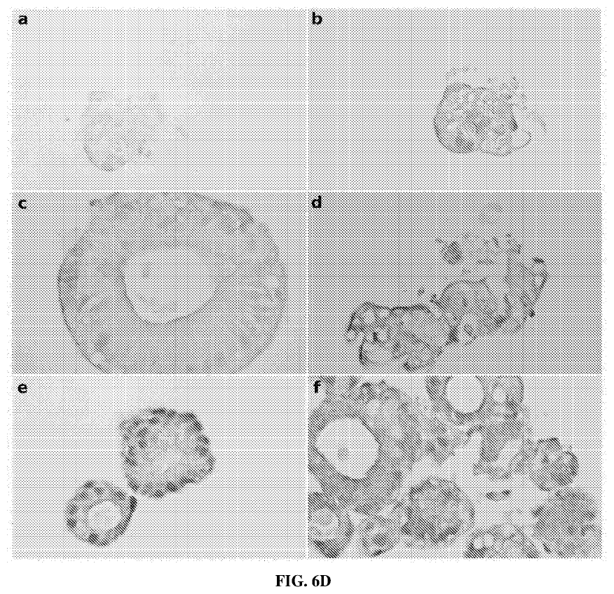

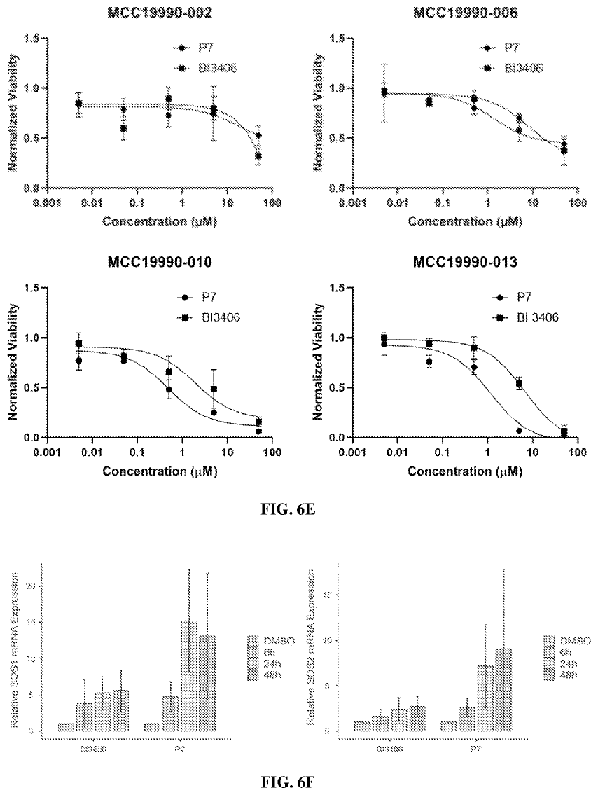

Our efficient synthesis of an effective and selective SOS1 degrader P7 allowed us to evaluate its ex vivo activity in CRC PDOs. We first looked at the effect of genetic SOS1 knockdown by siRNA compared to SOS1 inhibitor BI3406 in a BI3406-resistant cell line HCT116. Compared to BI3406, siSOS1 led to effective SOS1 knockdown which was associated with increased cellular apoptosis in the absence of pERK suppression (FIG. 6A). A RAS-MAPK pathway independent mechanism might be present in the cellular effect of SOS1 knockdown.³⁷ We hypothesized that SOS1 degradation by SOS1 degrader may be superior to inhibit cell proliferation and survival in CRC compared to SOS1 inhibitor. To test this hypothesis, CRC PDOs, MCC19990-002, MCC19990-010, and MCC19990-013, were treated with 1 μM P7 for 24 hours and stained for SOS1 expression. As shown in FIG. 6B, compared with DMSO control, treatment with P7 led to significant SOS1 degradation at varying degrees in different cellular compartments of these CRC PDOs. In BI3406-sensitive CRC PDO MCC19990-010, SOS1 degradation induced by 1 μM P7 for 24 hours led to significant necrosis and loss of cellular structures compared to DMSO control (FIG. 6C a,b). In contrast, in another BI3406-sensitive CRC PDO, MCC19990-013, P7-induced SOS1 degradation at 24 hours may not be sufficient to cause cell death but had initial phase of gland structure alteration in CRC PDO (FIG. 6C c,d). The kinetics of pharmacodynamic effects of P7 may vary in different CRC PDOs. The P7-induced morphologic changes of CRC PDOs were also supported by increased membranous expression of Annexin V in MCC19990-010 and MCC19990-013 after treatment with 1 μM P7 for 24 hours as compared to DMSO control (FIG. 6D). P7 demonstrated differential activities to inhibit proliferation and survival of KRAS-mutant CRC PDOs. MCC19990-002 has KRAS G12A mutation; MCC19990-006 has KRAS G12C mutation; MCC19990-010 has KRAS G12A mutation; MCC19990-013 has KRAS G12A mutation. As shown in FIG. 6E, MCC19990-002 was resistant to both P7 and BI3406. P7 had superior activity in MCC19990-006, MCC19990-010, and MCC19990-013 CRC PDOs. In MCC19990-006, P7 had an IC₅₀ of 1.4 μM; BI3406 had an IC₅₀ of 8.5 μM. In MCC19990-010, P7 had an IC₅₀ of 0.48 μM; BI3406 had an IC₅₀ of 1.9 μM. In MCC19990-013, P7 had an IC₅₀ of 1.16 μM; BI3406 had an IC₅₀ of 6.7 μM. Last but not least, as a result of SOS1 inhibition by BI3406 and SOS1 degradation by P7, both SOS1 and SOS2 were upregulated at the mRNA level in SW620 cells (FIG. 6F), which may serve as a potential cellular adaptation mechanism needed to be addressed in future drug discovery efforts. In summary, P7 demonstrated effective SOS1 degradation leading to morphologic alterations and apoptosis in ex vivo CRC PDOs. The cellular effect of P7-induced SOS1 degradation may be different in CRC PDOs, which was largely due to tumor heterogeneity. P7 had superior activity to SOS1 inhibitor BI3406 in SOS1 inhibition sensitive KRAS-mutant CRC PDOs.

### CONCLUSIONS

We reported the design and synthesis of a SOS1 degrader P7 which effectively targeted SOS1 for degradation through the ubiquitin-proteasome system. SOS1 degradation induced by P7 was time and concentration dependent with high specificity according to global proteomics analysis. P7 induced SOS1 degradation in multiple CRC cell lines and patient-derived PDO models. P7 demonstrated superior cytotoxic activity compared to BI3406 in SOS1 inhibitor sensitive CRC PDO models. These findings support further development of SOS1 degrader as a new class of agents targeting KRAS-driven CRC and as a tool to evaluate its activity either alone or in combination and to reveal cellular adaptation mechanisms to degraders in CRC.

### EXPERIMENTAL SECTION

### Chemistry

General Information. All chemicals were obtained from commercial suppliers and used as purchased without further purification. Reactions were monitored by LC/MS and thin layer chromatography (TLC). TLC was performed using SiliCycle Inc. silica plates, using short-wave UV light (254 nm, UVP, LLC) for visualization. Nuclear magnetic resonance (NMR) spectra were recorded on Bruker 400 and 500 MHz instruments and were calibrated using deuterated solvent (CDCl₃: ¹H NMR: 7.26 ppm, ¹³C NMR: 77.16 ppm, DMSO-d₆: ¹H NMR: 2.50 ppm, ¹³C NMR: 39.52 ppm, CD₃OD: ¹H NMR: 3.31 ppm, ¹³C NMR: 49.00 ppm). Data are reported as follows: chemical shift (δ), multiplicity, integrated intensity, and coupling constant (J) in hertz. Column chromatography was performed with silica gel (230-400 mesh) on the Yamazen A1580S EPCLC automated system. High performance liquid chromatography (HPLC) grade acetonitrile/water was obtained from Fisher Scientific International, Inc. High-resolution mass spectroscopy (HRMS) traces were obtained on an Agilent 6230 TOF/LC/MS. HPLC analysis was performed on an Agilent 1260 system using a ZORBAX C18 column (150×4.6 mm, 5 μm) at room temperature with a gradient elution using the mobile phase (A) nanopure water containing 0.1% formic acid and (B) acetonitrile containing 0.1% formic acid. All compounds used for biological evaluation have a purity of ≥95%.

Methyl 2-amino-4-(benzyloxy)-5-methoxybenzoate (3). A mixture of 2 (3.0 g, 9.5 mmol), Fe (4.3 g, 76 mmol), NH₄Cl (4.3 g, 80 mmol) in CH₃OH/H₂O (45.0 mJ/22.5 mL) was stirred at 105° C. for 4 h. The mixture was filtered through celite and washed with EtOAc. The organic layer was washed with brine, dried over Na₂SO₄ and concentrated to afford the desired product 3 (2.4 g, 89% yield) as a brown solid. ¹H NMR (400 MHz, CDCl₃): δ 7.43 (dd, J=8.1, 1.5 Hz, 2H), 7.40-7.36 (m, 2H), 7.36 (s, 1H), 7.34-7.30 (m, 1H), 6.40 (s, 1H), 5.15 (s, 2H), 3.86 (s, 3H), 3.84 (s, 3H).

6-Methoxy-2-methyl-7-(phenylmethoxy)-4(3H)-quinazolinone (4). A mixture of 3 (2.3 g, 8.0 mmol), acetonitrile (21 mL), 4 M HCl in dioxane (42 mL) was refluxed for 15 h.

The mixture was cooled and poured carefully into a cold NaHCO₃ (sat.) solution. The precipitate formed and was collected by filtration, washed extensively with water and air-dried to afford the desired product 4 (2.3 g, 96% yield) as a white solid. ¹H NMR (400 MHz, CDCl₃): δ 10.88 (s, 1H), 7.60 (s, 1H), 7.46 (d, J=7.1 Hz, 2H), 7.39 (t, J=7.3 Hz, 2H), 7.32 (t, J=7.2 Hz, 1H), 7.12 (s, 1H), 5.27 (s, 2H), 4.01 (s, 3H), 2.52 (s, 3H).

7-(benzyloxy)-6-methoxy-2-methylquinazolin-4-yl 2,4,6-triisopropylbenzenesulfonate (5). A mixture of 4 (748 mg, 2.53 mmol), 2,4,6-triisopropylbenzenesulfonyl chloride (918 mg, 3.03 mmol), DMAP (30.9 mg, 0.25 mmol) and Et₃N (772 mg, 7.58 mmol) was suspended in DCM (35 mL). The reaction mixture was stirred at room temperature for 2 days. The reaction was diluted with DCM, extracted with NaHCO₃ (sat.). The combined organic layers were dried over Na₂SO₄ and concentrated in vacuo. The crude product was purified by flash chromatography using hexane/EtOAc to afford the desired product 5 (532 mg, 51% yield), recovered 4 200 mg, as a white solid. ¹H NMR (400 MHz, CDCl₃): δ 7.46 (d, J=7.0 Hz, 2H), 7.41-7.35 (m, 2H), 7.35-7.29 (m, 2H), 7.25 (s, 1H), 7.20 (s, 2H), 5.27 (s, 2H), 4.30-4.37 (m, 2H), 4.01 (s, 3H), 2.88-2.95 (m, 1H), 2.49 (s, 3H), 1.25 (d, J=6.7 Hz, 18H).

General Procedure for Synthesis of Compounds 8 and 9. A mixture of 5 (1 equiv.), 6 or 7¹⁷ (1.3 equiv.) and Et₃N in DMSO was stirred at 90° C. for 12 h. The mixture was cooled to room temperature. The residue dissolved in DCM and extracted with water. The combined organic layers were dried over Na₂SO₄ and concentrated in vacuo. The crude product was purified by flash chromatography to afford the desired product as a yellow solid.

(R)-7-(benzyloxy)-6-methoxy-2-methyl-N-(1-phenylethyl)quinazolin-4-amine (8). ¹H NMR (400 MHz, CDCl₃): δ 7.45 (d, J=7.5 Hz, 2H), 7.41 (d, J=7.8 Hz, 2H), 7.33 (dd, J=5.7, 2.6 Hz, 4H), 7.28 (dd, J=2.5, 1.4 Hz, 1H), 7.19 (s, 1H), 6.97 (s, 1H), 5.68-5.75 (m, 1H), 5.20 (s, 2H), 3.90 (s, 3H), 2.56 (s, 3H), 1.67 (d, J=6.8 Hz, 3H).

(R)-7-(benzyloxy)-6-methoxy-2-methyl-N-(1-(3-nitro-5-(trifluoromethyl)phenyl)ethyl)q-uinazolin-4-amine (9). ¹H NMR (400 MHz, CDCl₃): δ 8.52 (s, 1H), 8.34 (s, 1H), 8.12 (s, 1H), 7.40 (d, J=7.4 Hz, 2H), 7.28-7.35 (m, 3H), 7.20 (s, 1H), 7.09 (s, 1H), 5.61-5.68 (m, 1H), 5.20 (s, 2H), 4.00 (s, 3H), 2.48 (s, 3H), 1.76 (d, J=6.5 Hz, 3H).

6-methoxy-7-benzyloxyquinazolin-4-ol (10). A mixture of 3 (431 mg, 1.50 mmol), ammonium formate (85.1 mg, 1.35 mmol) and formamide (3.6 mL) was stirred at 185° C. (oil bath temperature) for 1 h. The mixture was cooled to room temperature. The resulting precipitate was isolated, washed with water and dried to afford the desired product 10 (381 mg, quant.) as a brown solid. ¹H NMR (400 MHz, CDCl₃): δ 7.50-7.44 (m, 2H), 7.43-7.31 (m, 4H), 7.29 (s, 1H), 5.30 (s, 2H), 4.04 (s, 3H), 2.77 (s, 3H).

General Procedure for Synthesis of Compounds 11 and 12. A mixture of 10 (1 equiv.), phosphonitrilic chloride trimer (1 equiv.), DIPEA (5 equiv.) in MeCN were added to a nitrogen purged pressure tube. The reaction mixture was refluxed for 20 h as an activation time. The reaction was monitored by TLC. Then 6 or 14¹⁷ (2 equiv.) was added and the reaction mixture was refluxed for 16 h. After the mixture was concentrated under reduced pressure, the residue was purified by flash chromatography to afford the desired product as a yellow solid.

(R)-7-(benzyloxy)-6-methoxy-N-(1-phenylethyl)quinazolin-4-amine (11). ¹H NMR (400 MHz, CDCl₃): δ 7.55 (d, J=7.6 Hz, 2H), 7.45 (d, J=6.6 Hz, 2H), 7.40 (t, J=7.5 Hz, 2H), 7.35-7.29 (m, 6H), 7.18 (s, 1H), 5.60-5.67 (m, 1H), 5.17 (s, 2H), 4.05 (s, 3H), 1.76 (d, J=6.9 Hz, 3H).

(R)-7-(benzyloxy)-6-methoxy-N-(1-(3-(trifluoromethyl)phenyl)ethyl)quinazolin-4-amine (12). ¹H NMR (400 MHz, CDCl₃): δ 7.72 (s, 2H), 7.53 (d, J=7.8 Hz, 1H), 7.47 (d, J=8.2 Hz, 2H), 7.43 (d, J=7.9 Hz, 2H), 7.38 (t, J=7.5 Hz, 2H), 7.33-7.31 (m, 2H), 7.18 (s, 1H), 5.65-5.72 (m, 1H), 4.05 (s, 3H), 1.79 (d, J=6.9 Hz, 3H).

7-(benzyloxy)-4-chloro-6-methoxy-2-methylquinazoline (13). A mixture of 4 (200 mg, 0.67 mmol), POCl₃ (1.03 g, 6.75 mmol) in toluene (1 mL) was refluxed for 16 h. The reaction mixture was cooled to room temperature and azeotroped with toluene. The mixture was dissolved in DCM and added with NaHCO₃ (sat.) until a basic pH reached. The organic phase was collected by filtration, dried over Na₂SO₄ and concentrated to afford the desired product 13 (183 mg, 86% yield) as an orange solid. ¹H NMR (400 MHz, CDCl₃): δ 7.48 (d, J=7.4 Hz, 2H), 7.37 (dd, J=14.5, 8.6 Hz, 5H), 5.31 (s, 2H), 4.05 (s, 3H), 2.79 (s, 3H).

(R)-7-(benzyloxy)-6-methoxy-2-methyl-N-(1-(3-(trifluoromethyl)phenyl)ethyl)quinazolin-4-amine (15). A mixture of 13 (183 mg, 0.58 mmol), DIPEA (150 mg, 1.16 mmol), 14¹⁷ (143 mg, 0.76 mmol) in EtOH (3 mL) was added to a nitrogen purged pressure tube and then was stirred at 100° C. for 16 h. The mixture was cooled to room temperature. After the mixture was concentrated under reduced pressure, the residue was purified by flash chromatography to afford the desired product 15 (214 mg, 79% yield) as a yellow solid. ¹H NMR (400 MHz, CDCl₃): δ 7.72 (s, 1H), 7.66 (d, J=7.7 Hz, 1H), 7.49 (d, J=7.8 Hz, 1H), 7.44-7.37 (m, 3H), 7.35-7.27 (m, 3H), 7.24 (s, 1H), 7.17 (s, 1H), 5.66-5.73 (m, 1H), 5.18 (s, 2H), 3.95 (s, 3H), 2.53 (s, 3H), 1.71 (d, J=6.9 Hz, 3H).

Methyl 2-amino-5-iodo-4-methoxybenzoate (17). A mixture of 16 (10.0 g, 60 mmol), the concentrated sulfuric acid (18 mL) in methanol (100 mL) was refluxed for 22 h. The mixture was cooled to room temperature, quenched by NaHCO₃ (sat.) to pH 6-7 and then extracted with EtOAc. The organic layer was washed with brine, dried over Na₂SO₄, and concentrated to afford methyl 2-amino-4-methoxybenzoate (10.7 g, 98% yield) as a brown solid which was directly used for the next step without further purification. Methyl 2-amino-4-methoxybenzoate (725 mg, 4.00 mmol) was dissolved in EtOH (3.0 mL). Water (5.0 mL) and concentrated HCl (1.2 mL) was added and the mixture was cooled to 0° C. A solution of iodine monochloride (714 mg, 4.40 mmol) in concentrated HCl (0.36 mL) was added dropwise and the reaction mixture was stirred at room temperature for 16 h. Then the mixture was quenched with water and the precipitate was filtered off. The crude product was washed with hexane to give the desired product 17 (1.14 g, 95% yield) as a brown solid. ¹H NMR (400 MHz, CDCl₃): δ 8.21 (s, 1H), 6.07 (s, 1H), 3.85 (s, 3H), 3.84 (s, 3H).

4-chloro-6-iodo-7-methoxy-2-methylquinazoline (18). A mixture of 17 (307 mg, 1.00 mmol), methane sulfonic acid (0.52 mL) in CH₃CN (2.6 mL) was added to a nitrogen purged pressure tube. The reaction mixture was refluxed for 18 h. After cooled to room temperature, the mixture was poured into NaHCO₃ (sat.) solution and the precipitate filtered off to afford 6-iodo-7-methoxy-2-methylquinazolin-4(3H)-one as a brown solid (295 mg, 93% yield) which was directly used for next step without further purification.

6-iodo-7-methoxy-2-methylquinazolin-4(3H)-one (722 mg, 2.28 mmol) in POCl₃ (1.2 mL) and toluene (4.6 mL) was added Et₃N (0.7 mL) dropwise. The mixture was stirred at 75° C. for 3 h. The reaction was quenched with ice water, washed with NaHCO₃ (sat.) and extracted with EtOAc. The combined organic layers were dried over Na₂SO₄, filtered and concentrated in vacuo. The crude product was purified by flash chromatography to afford the desired product 18 (627 mg, 82% yield) as a yellow solid. ¹H NMR (400 MHz, CDCl₃): δ 8.66 (s, 1H), 7.21 (s, 1H), 4.05 (s, 3H), 2.80 (s, 3H).

(R)-6-iodo-7-methoxy-2-methyl-N-(1-(3-(trifluoromethyl)phenyl)ethyl)quinazolin-4-amine (19). A mixture of 18 (335 mg, 1.00 mmol), 14¹⁷ (246 mg, 1.30 mmol), DIPEA (259 mg, 2.00 mmol) in EtOH (3 mL) was added to a nitrogen purged pressure tube. The mixture was heated to 100° C. for 16 h. After cooled to room temperature and concentrated under reduced pressure, the residue was dissolved in EtOAc and washed with NaHCO₃ (sat.). The combined organic layers were dried over Na₂SO₄, filtered and concentrated in vacuo. The crude product was purified by flash chromatography to afford the desired product 19 (426 mg, 87% yield) as a yellow solid. ¹H NMR (400 MHz, CDCl₃): δ 8.15 (s, 1H), 7.72 (s, 1H), 7.63 (d, J=7.6 Hz, 1H), 7.53 (d, J=7.6 Hz, 1H), 7.46 (t, J=7.7 Hz, 1H), 7.11 (s, 1H), 5.63-5.70 (m, 1H), 3.96 (s, 3H), 2.55 (s, 3H), 1.70 (d, J=6.9 Hz, 3H).

(R)-tert-butyl 4-hydroxy-4-(7-methoxy-2-methyl-4-((1-(3-(trifluoromethyl)phenyl)ethyl)-amino)quinazolin-6-yl)piperidine-1-carboxylate (20). A mixture of 19 (24.5 mg, 0.05 mmol), DMPU (12.8 mg, 0.12 mmol) in dry THF was cooled to −78° C. Then isopropylmagnesium bromide (15.4 mg, 0.15 mmol) was added dropwise and the reaction mixture was stirred at −78° C. for 1 h. Boc-4-piperidone (14.9 mg, 0.08 mmol) was added and the reaction mixture was stirred at room temperature for 12 h. The reaction was quenched with NH₄CI (sat.) and extracted with DCM. The combined organic layers were dried over Na₂SO₄, filtered and concentrated in vacuo. The crude product was purified by flash chromatography to afford the desired product 22 (19.5 mg, 69% yield) as a yellow solid. ¹H NMR (400 MHz, CDCl₃): δ 7.78 (s, 1H), 7.74 (s, 1H), 7.67 (d, J=7.7 Hz, 1H), 7.52 (d, J=7.8 Hz, 1H), 7.44 (t, J=7.7 Hz, 1H), 7.31 (d, J=11.2 Hz, 1H), 5.70-5.78 (m, 1H), 4.01 (t, J=11.2 Hz, 2H), 3.96 (s, 3H), 3.26 (t, J=12.4 Hz, 2H), 2.59 (s, 3H), 2.13-2.04 (t, J=11.4 Hz, 2H), 1.91 (t, J=12.8 Hz, 2H), 1.74 (d, J=7.0 Hz, 3H), 1.49 (s, 9H).

General Procedure for Synthesis of Compounds 1a, 1b, 1c, and 1d. A mixture of 8 or 9 or 15 (1 equiv.) and 10 wt % Pd/C (10 mol %) in methanol was stirred under H₂ (1 atm) at room temperature for 3 h. After reaction completion detected by TLC, the mixture was filtered through celite and washed with EtOAc. Removal of organic solvent afforded the hydrogenation product as a yellow solid without further purification.

A mixture of hydrogenation product (1 equiv.), tert-butyl 5-bromopentanoate³⁸ (2 equiv.) and K₂CO₃ (1.2 equiv.) in DMF was stirred at 70° C. for 3 h. The reaction was quenched with water and extracted with DCM. The combined organic layers were dried over Na₂SO₄, filtered and concentrated in vacuo. The crude product was purified by flash chromatography to afford the desired product 21 or 22 or 23 as a yellow solid.

21 or 22 or 23 (1 equiv.) in DCM was treated with TFA (33 equiv.) and stirred at room temperature for 3 h. After reaction completion detected by TLC, the mixture was rinsed with DCM several times and concentrated under reduced pressure afforded the acid product as a yellow solid without further purification.

A mixture of acid (1 equiv.), EDCI (2 equiv.), OxymaPure (1.5 equiv.) and Et₃N (5 equiv.) in DMF was stirred at room temperature for 30 min. Ihalidomide-PEG2- -C2-NH2 hydrochloride³⁹ (2 equiv.) or VHL ligand 2 hydrochloride (2 equiv.) was added and was stirred at room temperature overnight. The reaction was quenched with NH₄Cl (sat.), and extracted with EtOAc. The combined organic layers were dried over Na₂SO₄, filtered and concentrated in vacuo. The crude product was purified by flash chromatography to afford the desired product 1a-1d as a yellow solid.

N-(2-(2-(2-((2-(2,6-dioxopiperidin-3-yl)-1,3-dioxoisoindolin-4-yl)amino)ethoxy)ethoxy)ethyl)-5-((6-methoxy-2-methyl-4-(((R)-1-phenylethyl)amino)quinazolin-7-yl)oxy)pentanamide (1a). ¹H NMR (400 MHz, CDCl₃): δ 7.50-7.43 (m, 2H), 7.40-7.38 (m, 1H), 7.29-7.27 (m, 1H), 7.22-7.19 (m, 1H), 7.18 (s, 1H), 7.00 (dd, J=9.4, 7.1 Hz, 1H), 6.81 (dd, J=8.6, 3.1 Hz, 1H), 6.68 (t, J=5.6 Hz, 1H), 6.40-6.44 (m, 1H), 5.70 (q, J=6.8 Hz, 1H), 4.80-4.89 (m, 1H), 3.97 (t, J=6.0 Hz, 2H), 3.87 (s, 3H), 3.65 (t, J=5.2 Hz, 2H), 3.59 (s, 4H), 3.55 (t, J=5.3 Hz, 2H), 3.36-3.44 (m, 4H), 2.84-2.62 (m, 3H). 2.57 (s, 3H), 2.25-2.19 (m, 2H), 2.02-2.09 (m, 1H), 1.71-1.79 (m, 4H), 1.67 (d, J=1.5 Hz, 3H). ¹³C NMR (101 MHz, CDCl₃): δ 173.27, 171.67, 169.46, 169.20, 167.65, 161.24, 157.99, 154.33, 149.00, 146.75, 143.56, 136.14, 132.46, 128.57, 127.39, 126.85, 116.85, 111.72, 110.18, 105.96, 101.76, 70.63, 70.21, 69.94, 69.29, 69.15, 56.71, 50.55, 48.93, 45.05, 42.34, 39.41, 37.33, 36.03, 35.53, 32.89, 31.49, 29.78, 27.74, 24.66, 22.86, 21.49. HRMS (ESI): m/z [M+H]⁺ calcd for C₄₂H₄₉N₇O₉, 796.3592; found, 796.3594.

N-(2-(2-(2-((2-(2,6-dioxopiperidin-3-yl)-1,3-dioxoisoindolin-4-yl)amino)ethoxy)ethoxy)ethyl)-5-((6-methoxy-2-methyl-4-(((R)-1-(3-(trifluoromethyl)phenyl)ethyl)amino)quinazolin-7-yl)oxy)pentanamide (1b). ¹H NMR (400 MHz, CDCl₃): 7.77-7.73 (m, 1H), 7.71-7.66 (m, 1H), 7.46 (d, J=7.9 Hz, 1H), 7.40-7.38 (m, 1H), 7.14 (s, 1H), 7.00-7.02 (m, 1H), 6.81 (d, J=8.6 Hz, 1H), 6.65 (s, 1H), 6.43 (q, J=5.2 Hz, 1H), 5.65-5.71 (m, 1H), 4.91-4.84 (m, 1H), 3.96 (d, J=6.1 Hz, 2H), 3.88 (s, 3H), 3.65-3.68 (m, 2H), 3.61 (s, 4H), 3.56 (t, J=5.2 Hz, 2H), 3.45-3.36 (m, 4H), 2.84-2.67 (m, 3H), 2.54 (d, J=2.3 Hz, 3H), 2.22 (q, J=6.3, 5.4 Hz, 2H), 2.11-2.04 (m, 1H), 1.81-1.72 (m, 4H), 1.71 (d, J=7.0 Hz, 3H). ¹³C NMR (101 MHz, CDCl₃): δ 173.30, 171.67, 169.51, 169.24, 167.70, 161.35, 157.90, 154.38, 149.05, 146.76, 144.90, 136.16, 132.47, 130.82, 130.49, 130.38, 129.05, 125.62, 124.12, 123.95, 122.92, 116.86, 111.75, 110.23, 106.03, 101.67, 70.68, 70.24, 69.95, 69.30, 69.16, 56.72, 50.42, 49.02, 42.36, 39.46, 36.06, 31.52, 29.81, 27.76, 24.79, 22.93, 21.65. HRMS (ESI): m/z [M+H]⁺ calcd for C₄₃H₄₈F₃N₇O₉, 864.3466; found, 864.3468.

S-((4-(((R)-1-(3-amino-5-(trifluoromethyl)phenyl)ethyl)amino)-6-methoxy-2-methylquinazolin-7-yl)oxy)-N-(2-(2-(2-((2-(2,6-dioxopiperidin-3-yl)-1,3-dioxoisoindolin-4-yl)amino)ethoxy)ethoxy)ethyl)pentanamide (1c). ¹H NMR (400 MHz, CDCl₃): δ 8.01 (s, 1H), 7.84 (s, 1H). 7.40 (d, J=9.2 Hz, 1H), 7.05-6.98 (m, 2H). 6.83 (d, J=6.2 Hz, 1H). 6.73 (s, 1H), 6.43 (d, J=7.5 Hz, 1H), 5.65 (s, 1H), 4.88 (s, 1H), 3.87-3.82 (m, 4H), 3.69 (t, J=4.9 Hz, 3H), 3.63 (s, 3H), 3.60-3.55 (m, 3H), 3.39-3.44 (m, 4H), 2.70 (d, J=11.4 Hz, 3H), 2.56 (d, J=8.7 Hz, 3H), 2.17 (s, 2H), 2.09 (s, 1H), 1.74-1.68 (m, 4H), 1.63 (d, J=5.1 Hz, 3H). ¹³C NMR (101 MHz, CDCl₃): δ 173.56, 171.79, 171.75, 169.44, 167.69, 162.70, 158.97, 158.33, 155.42, 149.79, 146.78, 136.22, 132.45, 116.97, 111.68, 110.19, 105.19, 70.64, 70.29, 69.81, 69.30, 69.26, 58.59, 57.09, 51.40, 51.24, 49.00, 42.37, 39.53, 36.64, 35.87, 32.07, 31.59, 31.51, 29.85, 27.78, 22.97, 22.95, 22.84, 21.11, 18.58, 14.26, 1.16. HRMS (ESI): m/z [M+H]⁺ calcd for C₄₃H₄₉F₃N₈O₉, 879.3575; found, 879.3582.

(2S,4R)-1-((S)-2-(5-((4-(((R)-1-(3-amino-5-(trifluoromethyl)phenyl)ethyl)amino)-6-methoxy-2-methylquinazolin-7-yl)oxy)pentanamido)-3,3-dimethylbutanoyl)-4-hydroxy-N—((S)-1-(4-(4-methylthiazol-S-yl)phenyl)ethyl)pyrrolidine-2-carboxamide (1d). ¹H NMR (400 MHz, CDCl₃): δ 8.65 (s, 1H), 7.54 (s, 2H), 7.39-7.30 (m, 4H), 7.08 (s, 1H), 7.02 (s, 1H), 6.71 (s, 1H), 5.63 (s, 1H), 5.10-5.02 (m, 1H), 4.71 (d, J=9.3 Hz, 1H), 4.61 (d, J=8.7 Hz, 1H), 4.47 (s, 1H), 4.06 (d, J=11.2 Hz, 2H), 3.84 (s, 3H), 3.60 (t, J=11.5 Hz, 2H), 2.50 (s, 2H), 2.48 (s, 3H), 2.35-2.26 (m, 2H), 2.24-2.12 (m, 2H), 1.74-1.64 (m, 5H), 1.45 (d, J=6.9 Hz, 3H), 1.25 (s, 3H), 1.02 (s, 9H). ¹³C NMR (101 MHz, DMSO-d₆): δ 171.87, 170.67, 169.60, 157.95, 154.71, 151.52, 149.55, 149.16, 147.77, 144.69, 131.15, 131.08, 129.71, 128.84, 126.41, 115.20, 109.66, 109.63, 108.39, 108.34, 105.12, 103.62, 103.59, 68.78, 68.55, 62.04, 58.58, 56.76, 56.46, 56.30, 49.84, 48.61, 47.73, 37.75, 35.21, 34.45, 29.05, 27.85, 26.47, 25.50, 24.84, 23.86, 22.45, 22.03, 21.23, 16.00, 13.97. HRMS (ESI): m/z [M+H]⁺ calcd for C₄₇H₅₇F₃N₈O₆S, 919.4074; found, 919.4071.

General Procedure for Synthesis of Compounds 1e, 1f and 1k-1m. A mixture of 8 or 9 or 11 or 12 (1 equiv.) and 10 wt % Pd/C (10 mol %) in Methanol was stirred under H₂ (1 atm) at room temperature for 3 h. After reaction completion detected by TLC, the mixture was filtered through celite and washed with EtOAc. Remove organic solvent to afford the hydrogenation product as a yellow solid without further purification.

A mixture of hydrogenation product (1 equiv.), 3-((tert-butoxycarbonyl)amino)propyl 4-methylbenzenesulfonate⁴⁰ or 17-azido-3,6,9,12,15-pentaoxaheptadecyl 4-methylbenzenesulfonate ⁴¹or 2-((tert-butoxycarbonyl)amino)ethyl methanesulfonate (same way as 3-((tert-butoxycarbonyl)amino)propyl 4-methylbenzenesulfonate) (2 equiv.) and K₂CO₃ (1.2 equiv.) in DMF was stirred at 75° C. overnight. The reaction was quenched with water and extracted with EtOAc. The combined organic layers were dried over Na₂SO₄, filtered and concentrated in vacuo. The crude product was purified by flash chromatography to afford the desired product 24-28 as a yellow solid.

24 or 26-28 (1 equiv.) in dioxane was treated with 4 M HCl in dioxane and stirred at room temperature for 1 h. After reaction completion detected by TLC, the mixture was rinsed with DCM several times and concentrated under reduced pressure afforded the amine product as a yellow solid without further purification.

A mixture of 25 (1 equiv.) and 10 wt % Pd/C (10 mol %) in Ethanol was stirred under H₂ (1 atm) at room temperature for 6 h. After reaction completion detected by TLC, the mixture was filtered through celite and washed with EtOAc. Remove organic solvent to afford the amine product as a yellow solid without further purification.

A mixture of amine 24-28 (1 equiv.), DIEA, and thalidomide 4-fluoride³⁹ in DMF was stirred at 75° C. overnight. The reaction was quenched with H₂O, and extracted with EtOAc. The combined organic layers were dried over Na₂SO₄, filtered and concentrated in vacuo. The crude product was purified by flash chromatography to afford the desired product 1e, 1f and 1k-1m as a yellow solid.

4-((3-((4-(((R)-1-(3-amino-5-(trifluoromethyl)phenyl)ethyl)amino)-6-methoxy-2-methylquinazolin-7-yl)oxy)propyl)amino)-2-(2,6-dioxopiperidin-3-yl)isoindoline-1,3-dione (1e). ¹H NMR (400 MHz, CD₃OD): δ 7.80 (s, 1H), 7.48-7.42 (m, 1H), 7.10 (d, J=8.6 Hz, 1H), 6.95-6.99 (m, 4H), 6.83 (s, 1H), 5.74-5.68 (m, 1H), 5.05-5.00 (m, 1H), 4.27 (t, J=5.6 Hz, 2H), 4.01 (s, 3H), 3.62-3.57 (m, 2H), 2.86-2.79 (m, 1H), 2.69-2.73 (m, 1H), 2.56 (s, 3H), 2.53-2.47 (m, 1H), 2.25-2.19 (m, 2H), 2.05-2.09 (m, 1H), 1.69 (d, J=7.1 Hz, 3H). ¹³C NMR (126 MHz, CD₃OD): δ 174.61, 171.77, 170.62, 169.22, 161.21, 159.98, 156.92, 151.59, 150.42, 148.11, 146.61, 141.61, 139.07, 137.14, 133.86, 132.45, 126.93, 124.77, 117.94, 117.10, 112.55, 111.86, 111.18, 106.82, 103.72, 102.27, 68.01, 57.15, 52.02, 40.20, 32.17, 29.76, 23.77, 23.03, 21.38. HRMS (ESI): m/z [M+H]⁺ calcd for C₃₅H₃₄F₃N₇O₆, 706.2523; found, 706.2523.

4-((17-((4-(((R)-1-(3-amino-S-(trifluoromethyl)phenyl)ethyl)amino)-6-methoxy-2-methylquinazolin-7-yl)oxy)-3,6,9,12,15-pentaoxaheptadecyl)amino)-2-(2,6-dioxopiperidin-3-yl)isoindoline-1,3-dione (if). ¹H NMR (400 MHz, CDCl₃): δ 7.48-7.40 (m, 1H), 7.18 (s, 1H), 7.10-7.00 (m, 2H), 6.98 (s, 1H), 6.87 (dd, J=8.5, 2.1 Hz, 1H), 6.76 (s, 1H), 6.41 (s, 1H), 4.90-4.84 (m, 1H), 3.95 (d, J=3.1 Hz, 1H), 3.93 (s, 3H), 3.66 (dd, J=10.5, 5.1 Hz, 9H), 3.59-3.47 (m, 10H), 3.43-3.37 (m, 4H), 3.35 (s, 1H), 2.89-2.78 (m, 4H), 2.76-2.67 (m, 2H), 2.10-2.06 (m, 1H), 1.70 (d, J=7.0 Hz, 3H). ¹³C NMR (126 MHz, CDCl₃): δ 171.31, 171.29, 169.39, 169.37, 168.70, 168.65, 167.73, 157.94, 154.20, 149.00, 146.90, 136.18, 132.57, 132.54, 131.48, 131.21, 127.76, 125.54, 123.37, 116.91, 111.79, 110.55, 110.33, 70.99, 70.70, 70.58, 70.50, 70.38, 69.88, 69.58, 68.62, 68.30, 66.76, 56.53, 49.00, 42.47, 32.06, 31.54, 29.84, 29.50, 25.86, 22.88, 14.25, 1.15. HRMS (ESI): m/z [M+H]⁺ calcd for C₄₄H₅₂F₃N₇O₁₁, 912.3677; found, 912.3667.

2-(2,6-dioxopiperidin-3-yl)-4-((2-((6-methoxy-4-(((R)-1-phenylethyl)amino)quinazolin-7-yl)oxy)ethyl)amino)isoindoline-1,3-dione (1k). ¹H NMR (400 MHz, CD₃OD): δ 8.57 (s, 1H), 7.89 (s, 1H), 7.55 (t, J=7.8 Hz, 1H), 7.46 (d, J=7.6 Hz, 2H), 7.35 (t, J=6.6 Hz, 2H), 7.25 (d, J=8.6 Hz, 2H), 7.15 (s, 1H), 7.04 (d, J=6.9 Hz, 1H), 5.83 (d, J=7.6 Hz, 1H), 4.97-5.04 (m, 1H), 4.41 (d, J=4.8 Hz, 2H), 3.99 (s, 3H), 3.86 (d, J=4.8 Hz, 2H), 2.85-2.76 (m, 1H), 2.73 (s, 1H), 2.66 (d, J=18.5 Hz, 1H), 2.05 (s, 1H), 1.74 (d, J=7.0 Hz, 3H). ¹³C NMR (126 MHz, CD₃OD): δ 174.57, 171.67, 170.60, 169.11, 160.31, 157.48, 152.61, 149.60, 147.97, 143.77, 137.08, 135.52, 133.74, 129.73, 128.64, 127.60, 118.68, 112.37, 111.56, 108.37, 104.27, 101.63, 70.25, 57.27, 52.89, 50.17, 42.64, 32.17, 30.74, 30.45, 23.77, 21.38. HRMS (ESI): m/z [M+H]⁺ calcd for C₃₂H₃₀N₆O₆, 595.2227; found, 595.2250.

2-(2,6-dioxopiperidin-3-yl)-4-((2-((6-methoxy-4-(((R)-1-(3-(trifluoromethyl)phenyl)ethyl)amino)quinazolin-7-yl)oxy)ethyl)amino)isoindoline-1,3-dione (11). ¹H NMR (400 MHz, CDCl₃): δ 8.45 (s, 1H), 7.67 (s, 1H), 7.63 (d, J=7.6 Hz, 1H), 7.48 (d, J=7.9 Hz, 1H), 7.41 (s, 1H), 7.24 (s, 1H), 7.12 (s, 1H), 7.02 (t, J=6.1 Hz, 1H), 6.93 (d, J=8.6 Hz, 1H), 6.55 (d, J=5.9 Hz, 1H), 5.67 (q, J=6.9 Hz, 1H), 4.80-4.91 (m, 1H), 4.27 (d, J=6.1 Hz, 2H), 3.87 (s, 3H), 3.70 (d, J=5.9 Hz, 2H), 2.80 (s, 1H), 2.70 (dd, J=11.8, 6.3 Hz, 2H), 2.09-2.02 (m, 1H), 1.68 (d, J=4.2 Hz, 3H). ¹³C NMR (101 MHz, CDCl₃): δ 171.59, 169.38, 169.07, 167.67, 162.70, 157.61, 153.57, 149.54, 146.63, 144.89, 136.07, 132.38, 131.09, 130.77, 130.25, 129.19, 125.59, 124.25, 122.88, 117.10, 112.08, 110.53, 108.91, 100.44, 67.82, 56.40, 50.02, 48.94, 41.79, 36.64, 31.50, 22.88, 22.05. HRMS (ESI): m/z [M+H]⁺ calcd for C₃₃H₂₉F₃N₆O₆, 663.2101; found, 663.2100.

2-(2,6-dioxopiperidin-3-yl)-4-((2-((6-methoxy-2-methyl-4-(((R)-1-phenylethyl)amino)quinazolin-7-yl)oxy)ethyl)amino)isoindoline-1,3-dione (1m). ¹H NMR (400 MHz, CDCl₃): δ 7.46 (d, J=7.7 Hz, 3H), 7.31 (t, J=7.5 Hz, 2H), 7.22 (s, 1H), 7.12 (s, 1H), 7.04 (t, J=8.0 Hz, 1H), 6.98 (dd, J=8.6, 5.5 Hz, 1H), 6.57-6.48 (m, 1H), 5.74-5.64 (m, 1H), 4.93-4.77 (m, 1H), 4.26 (d, J=5.1 Hz, 2H), 3.88 (s, 3H), 3.72 (s, 2H), 2.82 (dd, J=13.0, 5.4 Hz, 1H), 2.76-2.64 (m, 2H), 2.57 (s, 3H), 2.11-2.00 (m, 1H), 1.69 (d, J=6.9 Hz, 3H). ¹³C NMR (126 MHz, CDCl₃): δ 171.40, 169.31, 168.93, 167.57, 157.92, 154.00, 149.30, 146.63, 143.41, 136.14, 132.41, 128.71, 127.56, 126.86, 117.25, 112.07, 110.51, 106.51, 101.74, 67.99, 67.23, 56.80, 50.59, 48.98, 41.83, 31.48, 29.84, 24.83, 22.86, 21.58, 18.58, 14.26, 1.15. HRMS (ESI): m/z [M+H]⁺ calcd for C₃₃H₃₂N₆O₆, 609.2383; found, 609.2366.

2-((4-(((R)-1-(3-amino-S-(trifluoromethyl)phenyl)ethyl)amino)-6-methoxy-2-methylquinazolin-7-yl)oxy)-N-(2-(2,6-dioxopiperidin-3-yl)-1-oxoisoindolin-4-yl)acetamide (1g). A mixture of 9 (1 equiv.) and 10 wt % Pd/C (10 mol %) in methanol was stirred under H₂ (1 atm) at room temperature for 3 h. After reaction completion detected by TLC, the mixture was filtered through celite and washed with EtOAc. Remove organic solvent to afford the hydrogenation product as a yellow solid without further purification.

A mixture of hydrogenation product (27.9 mg, 0.07 mmol), KI (1.18 mg, 0.01 mmol), KHCO₃ (21.4 mg, 0.21 mmol) and 2-bromo-N-(2-(2,6-dioxopiperidin-3-yl)-1-oxoisoindolin-4-yl)acetamide⁴² (32.5 mg, 0.09 mmol) in DMF (1 mL) was stirred at 60° C. for 12 h. The reaction was quenched with water and extracted with EtOAc. The organic layer was washed with brine, dried over Na₂SO₄, filtered and concentrated in vacuo. The crude product was purified by flash chromatography to afford the desired product 1g (15.1 mg, 30%) as a white solid. ¹H NMR (400 MHz, DMSO-d₆): δ 11.03 (d, J=2.9 Hz, 1H), 10.24 (s, 1H), 7.87-7.77 (m, 2H), 7.57-7.50 (m, 2H), 7.04 (s, 1H), 6.87 (d, J=12.8 Hz, 2H), 6.71 (s, 1H), 5.63-5.57 (m, 1H), 5.56 (s, 1H), 5.14 (dd, J=12.4, 4.8 Hz, 1H), 4.97 (s, 1H), 4.37 (d, J=6.0 Hz, 1H), 4.32 (d, J=3.9 Hz, 1H), 3.96 (s, 3H), 2.86-2.97 (m, 1H), 2.58 (t, J=15.3 Hz, 1H), 2.39 (s, 3H), 2.27-2.16 (m, 1H), 2.04-1.97 (m, 1H), 1.58 (d, J=7.1 Hz, 3H). ¹³C NMR (126 MHz, DMSO-d₆): δ 172.85, 171.04, 167.75, 166.04, 157.71, 149.44, 134.28, 134.16, 132.90, 132.80, 129.82, 129.58, 128.89, 125.69, 125.54, 123.53, 119.77, 119.72, 115.10, 115.06, 109.62, 108.09, 106.51, 103.07, 69.80, 67.24, 56.42, 51.49, 46.30, 31.25, 30.72, 22.64, 22.62, 21.63. HRMS (ESI): m/z [M+H]⁺ calcd for C₃₄H₃₂F₃N₇O₆, 692.2366; found, 692.2361.

4-(((R)-1-(3-amino-5-(trifluoromethyl)phenyl)ethyl)amino)-6-methoxy-2-methylquinazolin-7-yl (2-(2,6-dioxopiperidin-3-yl)-1-oxoisoindolin-4-yl)carbamate (1h). A mixture of 9 (1 equiv.) and 10 wt % Pd/C (10 mol %) in methanol was stirred under H₂ (1 atm) at room temperature for 3 h. After reaction completion detected by TLC, the mixture was filtered through celite and washed with EtOAc. Remove organic solvent to afford the hydrogenation product as a yellow solid without further purification.

A mixture of hydrogenation product (14.3 mg, 0.04 mmol), DIEA (5.89 mg, 0.05 mmol) and 4-nitrophenyl (2-(2,6-dioxopiperidin-3-yl)-1-oxoisoindolin-4-yl)carbamate⁴³ (77.3 mg, 0.18 mmol) in DMF was stirred at room temperature for 3 days. The reaction was quenched with water and extracted with EtOAc. The organic layer was washed with brine, dried over Na₂SO₄, filtered and concentrated in vacuo. The crude product was purified by flash chromatography to afford the desired product 1h (30.5 mg, 90% yield) as a white solid. ¹H NMR (400 MHz, CD₃OD): δ 8.07-8.00 (m, 1H), 7.88 (d, J=7.0 Hz, 1H), 7.80 (d, J=8.6 Hz, 1H), 7.76 (d, J=2.6 Hz, 1H), 7.51-7.48 (m, 1H), 7.46 (dd, J=7.7, 2.2 Hz, 1H), 7.42 (s, 1H), 6.82 (s, 1H), 5.77 (q, J=7.0 Hz, 1H), 5.16-5.12 (m, 1H), 4.57-4.47 (m, 2H), 3.98 (s, 3H), 3.66 (d, J=3.3 Hz, 1H), 2.88 (dd, J=11.7, 6.0 Hz, 1H), 2.79-2.72 (m, 1H), 2.54 (s, 3H), 2.22-2.13 (m, 1H), 1.74 (d, J=7.1 Hz, 3H). ¹³C NMR (101 MHz, CD₃OD): δ 174.70, 172.17, 171.32, 160.14, 159.70, 154.66, 152.06, 147.20, 141.75, 139.23, 135.60, 133.54, 132.11, 130.15, 124.77, 124.21, 121.17, 119.26, 118.29, 114.99, 104.17, 103.24, 78.33, 56.98, 56.27, 53.63, 51.88, 32.34, 30.69, 24.00, 22.66, 21.54, 19.30. HRMS (ESI): m/z [M+H]⁺ calcd for C₃₃H₃₀F₃N₇O₆, 678.2210; found, 678.2210.

4-(((4-(((R)-1-(3-amino-S-(trifluoromethyl)phenyl)ethyl)amino)-6-methoxy-2-methylquinazolin-7-yl)oxy)methyl)-2-(2,6-dioxopiperidin-3-yl)isoindoline-1,3-dione (i). A mixture of 9 (1 equiv.) and 10 wt % Pd/C (10 mol %) in methanol was stirred under H₂ (1 atm) at room temperature for 3 h. After reaction completion detected by TLC, the mixture was filtered through celite and washed with EtOAc. Remove organic solvent to afford the hydrogenation product as a yellow solid without further purification.

A mixture of hydrogenation product (20.0 mg, 0.05 mmol), 4-(bromomethyl)-2-(2,6-dioxopiperidin-3-yl)isoindoline-1,3-dione⁴⁴ (35.8 mg, 0.10 mmol) and 60 wt % NaH (2.45 mg, 0.06 mmol) in DMF (1 mL) was stirred at 100° C. for 5 h. The reaction was quenched with water and extracted with DCM. The combined organic layers were dried over Na₂SO₄, filtered and concentrated in vacuo. The crude product was purified by flash chromatography to afford the desired product 1i (8.40 mg, 25% yield) as a white solid. ¹H NMR (400 MHz, CDCl₃): δ 7.90 (d, J=7.7 Hz, 1H), 7.71 (t, J=6.6 Hz, 1H), 7.67-7.62 (m, 1H), 7.14 (s, 1H), 7.08 (d, J=7.2 Hz, 1H), 7.01 (s, 1H), 6.97 (d, J=5.7 Hz, 1H), 6.78 (s, 1H), 5.57 (s, 1H), 5.30 (s, 4H), 4.95-5.01 (m, 1H), 3.97 (s, 3H), 2.96-2.86 (m, 2H), 2.78 (d, J=14.1 Hz, 1H), 2.54 (s, 3H), 2.22-2.15 (m, 1H), 1.66 (d, J=6.8 Hz, 4H). ¹³C NMR (126 MHz, DMSO-d₆): δ 172.80, 169.83, 167.17, 166.88, 157.82, 149.48, 135.15, 133.73, 131.78, 129.62, 128.41, 128.24, 127.79, 127.22, 125.67, 123.51, 123.17, 115.13, 109.62, 108.19, 103.38, 65.28, 56.61, 48.98, 33.57, 30.95, 29.02, 28.81, 24.75, 21.99, 21.47, 13.92, 2.00. HRMS (ESI): m/z [M+H]⁺ calcd for C₃₃H₂₉F₃N₆O₆, 663.2101; found, 663.2100.

2-(2,6-dioxopiperidin-3-yl)-4-(((6-methoxy-2-methyl-4-(((R)-1-phenylethyl)amino)quinazolin-7-yl)oxy)methyl)isoindoline-1,3-dione (1j). A mixture of 8 (1 equiv.) and 10 wt % Pd/C (10 mol %) in methanol was stirred under H₂ (1 atm) at room temperature for 3 h. After reaction completion detected by TLC, the mixture was filtered through celite and washed with EtOAc. Remove organic solvent to afford the hydrogenation product as a yellow solid without further purification.

A mixture of hydrogenation product (31.8 mg, 0.11 mmol), 4-(bromomethyl)-2-(2,6-dioxopiperidin-3-yl)isoindoline-1,3-dione (24.1 mg, 0.07 mmol) and K₂CO₃ (11.4 mg, 0.08 mmol) in DMF (1 mL) was stirred at 60° C. overnight. The reaction was quenched with water and extracted with DCM. The combined organic layers were dried over Na₂SO₄, filtered and concentrated in vacuo. The crude product was purified by flash chromatography to afford the desired product 1j (14.3 mg, 36% yield) as a white solid. ¹H NMR (400 MHz, DMSO-d₆): δ 11.14 (s, 1H), 8.25 (s, 1H), 7.93 (d, J=4.1 Hz, 2H), 7.81 (s, 1H), 7.45 (d, J=7.7 Hz, 2H), 7.33 (t, J=7.5 Hz, 2H), 7.22 (t, J=7.3 Hz, 1H), 7.09 (s, 1H), 5.66 (s, 2H), 5.17 (dd, J=12.9, 5.4 Hz, 1H), 4.11 (d, J=2.9 Hz, 1H), 3.94 (s, 3H), 2.88 (dd, J=13.2, 5.0 Hz, 1H), 2.61 (d, J=16.2 Hz, 1H), 2.36 (s, 3H), 2.12-2.05 (m, 1H), 2.03-1.96 (m, 1H), 1.60 (d, J=7.0 Hz, 3H). ¹³C NMR (101 MHz, DMSO-d₆): δ 172.77, 169.81, 167.16, 166.88, 161.13, 157.69, 152.58, 148.01, 144.66, 135.49, 135.06, 133.71, 131.75, 128.23, 127.73, 126.64, 126.28, 123.04, 106.61, 102.89, 73.26, 68.87, 65.06, 61.93, 56.40, 48.85, 33.56, 31.14, 28.86, 24.74, 21.98, 20.65. HRMS (ESI): m/z [M+H]⁺ calcd for C₃₂H₂₉N₅O₆, 580.2118; found, 580.2125.

General Procedure for Synthesis of Compounds 1n and 1o. Compound 20 in dioxane was treated with 4 M HCl in dioxane and stirred at room temperature for 3 h. After reaction completion detected by TLC, the mixture was rinsed with DCM several times and concentrated under reduced pressure afforded the amine product as a yellow solid without further purification.

A mixture of 3-((2-(2,6-dioxopiperidin-3-yl)-1,3-dioxoisoindolin-4-yl)amino)propanoic acid⁴⁵ or 2-((2-(2,6-dioxopiperidin-3-yl)-1,3-dioxoisoindolin-4-yl)amino)acetic acid (2 equiv.), EDCI (2 equiv.), OxymaPure (1.5 equiv.) and Et₃N (5 equiv.) in DMF was stirred at room temperature for 30 min. The amine product (1 equiv.) was added and was stirred at room temperature overnight. The reaction was quenched with NH₄Cl (sat.), and extracted with EtOAc. The combined organic layers were dried over Na₂SO₄, filtered and concentrated in vacuo. The crude product was purified by flash chromatography to afford the desired product in and 1o as a yellow solid.

2-(2,6-dioxopiperidin-3-yl)-4-((3-(4-hydroxy-4-(7-methoxy-2-methyl-4-(((R)-1-(3-(trifluoromethyl)phenyl)ethyl)amino)quinazolin-6-yl)piperidin-1-yl)-3-oxopropyl)amino)isoindoline-1,3-dione (In). ¹H NMR (400 MHz, CDCl₃): δ 7.99 (s, 1H), 7.73 (s, 1H), 7.65 (s, 1H), 7.46 (d, J=8.4 Hz, 1H), 7.41 (d, J=7.4 Hz, 1H), 7.20 (s, 1H), 7.01 (d, J=7.1 Hz, 1H), 6.89 (t, J=7.3 Hz, 1H), 6.62-6.47 (m, 1H), 5.76-5.64 (m, 1H), 4.81 (d, J=37.3 Hz, 1H), 4.45 (s, 1H), 3.87 (s, 3H), 3.54 (d, J=32.0 Hz, 4H), 3.01 (s, 1H), 2.82-2.70 (m, 2H), 2.69-2.59 (m, 3H), 2.54 (s, 3H), 2.07 (d, J=32.3 Hz, 3H), 1.80 (t, J=17.0 Hz, 2H), 1.68 (d, J=7.0 Hz, 3H). ¹³C NMR (101 MHz, CDCl₃): δ 171.79, 169.51, 169.22, 167.75, 163.76, 161.45, 158.85, 146.65, 144.63, 136.34, 135.37, 132.62, 130.51, 130.35, 129.09, 125.61, 124.21, 123.90, 122.91, 120.02, 116.72, 111.69, 110.19, 106.29, 71.92, 56.17, 50.38, 49.00, 41.69, 38.66, 37.89, 36.04, 35.31, 32.62, 31.52, 29.83, 22.82, 21.51, 14.25, 1.15. HRMS (ESI): m/z [M+H]⁺ calcd for C₄₀H₄₀F₃N₇O₇, 788.2941; found, 788.2957.

2-(2,6-dioxopiperidin-3-yl)-4-((2-(4-hydroxy-4-(7-methoxy-2-methyl-4-(((R)-1-(3-(trifluoromethyl)phenyl)ethyl)amino)quinazolin-6-yl)piperidin-)-yl)-2-oxoethyl)amino)isoindoline-1,3-dione (10). ¹H NMR (400 MHz, CD₃OD): δ 8.46-8.42 (m, 1H), 7.78 (s, 1H), 7.72 (d, J=7.3 Hz, 1H), 7.51 (q, J=8.1, 6.6 Hz, 3H), 7.10-6.94 (m, 3H), 5.75-5.81 (m, 1H), 5.04 (dd, J=12.5, 5.4 Hz, 1H), 4.48 (d, J=12.9 Hz, 1H), 4.24-4.17 (m, 1H), 4.01 (s, 1H), 3.95 (s, 3H), 3.85 (s, 1H), 3.72-3.64 (m, 1H), 3.35 (s, 1H), 3.25 (d, J=12.5 Hz, 1H), 2.89-2.80 (m, 1H), 2.76-2.65 (m, 2H), 2.65-2.54 (m, 1H), 2.52 (s, 3H), 2.13-2.05 (m, 1H), 1.89 (d, J=14.0 Hz, 1H), 1.79 (d, J=14.0 Hz, 1H), 1.70 (d, J=2.1 Hz, 3H). ¹³C NMR (101 MHz, CD₃OD): δ 174.65, 171.64, 170.47, 169.29, 168.48, 164.36, 163.40, 160.61, 147.02, 146.54, 138.06, 137.24, 133.80, 131.48, 130.32, 127.06, 124.93, 124.54, 122.28, 118.61, 112.22, 111.70, 107.18, 103.78, 72.71, 56.45, 53.53, 51.65, 44.95, 42.00, 39.73, 36.61, 35.94, 32.21, 30.70, 25.25, 24.37, 23.77, 21.56. HRMS (ESI): m/z [M+H]⁺ calcd for C₃₉H₃₈F₃N707, 774.2785; found, 774.2782.

Crystal structure informed design of SOS1 PROTAC degraders Coordinates for crystal structures of SOS1 in a complex with BI68BS (PDB: 6SFR) with a resolution of 1.92 Å¹⁷ and cereblon in a complex with cereblon E3 ligase (PDB: 4TZ4) with a resolution of 3.01 Å⁴⁶ were retrieved from the RCSB Protein Data Bank. They were prepared using the protein preparation wizard in Maestro of Schrödinger Release 2020-3.²⁶ In brief, hydrogens were added to all atoms; bond order and formal charges were added to heterogroups. Water molecules were not involved in protein-ligand interactions, thus were removed. To optimize the hydrogen bond interactions, hydroxyl and thiol hydrogens were sampled, 1800 rotations of the terminal angle of Asn, Gln, and His residues were assigned, and His tautomers and ionization states were predicted. The conformation of missing side-chain atoms if present were predicted. A brief relaxation was performed using an all-atom constrained minimization to reduce steric clashes present in the original crystal structures.⁴⁷ Structural interactions between SOS1 and BI68BS were outlined by the structural interaction fingerprint analysis function in Maestro.²⁶ The prepared protein structures were used for protein-protein docking experiments in Rosetta 3.12.²⁹ Protein-protein local docking protocol was applied to the structure coordinates in an input pdb file prepared for docking. The following scripts were used to generate 500 docking poses, from which total score and interface score were retrieved from the output file. The distance between /A LBK 1103 013 (BI68BS 6-methoxy group) and /C LVY 502 N17 (lenalidomide amino group) and the distance between /A LBK 1103 014 (BI68BS 7-methoxy group) and /C LVY 502 N17 were measured. Molecular graphics and analyses performed with UCSF ChimeraX⁴⁸, developed by the Resource for Biocomputing, Visualization, and Informatics at the University of California, San Francisco, with support from National Institutes of Health R01-GM129325 and the Office of Cyber Infrastructure and Computational Biology, National Institute of Allergy and Infectious Diseases. Statistical analyses were performed using R packages⁴⁹ and jamovi⁵⁰.

$ROSETTA3/bin/docking_protocol.static.linuxgccrelease
@flag_local_docking
flag_local_docking:
-in:file:s 6SFR_4TZ4_prepared.pdb
-unboundrot 6SFR_4TZ4_prepared.pdb
-nstruct 500
-partners A_C
-dock_pert 3 8
-ex1
-ex2aro
-out:path:all output_files
-out:suffix _local_dock

### Cell Line Culture

SW620, HCT116, C2BB, and SW1417 were purchased from the American Type Culture Collection. The cells were cultured in modified McCoys' 5A medium (Gibco, 16600082) supplemented with 10% FBS (ATCC, 30-2020) and 1% penicillin-streptomycin (ATCC, 30-2300). They were incubated under a 5% (v/v) CO2 atmosphere at 37° C.

### Patients

Patients were enrolled in one of several prospective studies approved for surgically resected tumor specimen collection at Moffitt Cancer Center (MCC). Tumor specimens used in this study were from either primary or metastatic CRC including but not limited to liver and peritoneal metastases as part of routine clinical care. These were approved under an umbrella protocol MCC20880 by the Institutional Review Board at MCC. Following collection of specimens, they were de-identified and assigned a lab number. Clinical information including, type and site of tumor specimen, patient's treatment history, previous genetic information, and organoid initiation date were collected when available. Patient-derived xenografts (MCC IACUC approval: A4100-01) were used to generate and biobank tumor specimens. The tumor samples were subsequently used to generate CRC PDOs.

### Patient-Derived Organoid Culture

CRC organoids were generated and expanded using a protocol similar to previously published ones for CRC with modifications.^{51,52} In brief, the tumor specimen was placed in PBS and then fresh wash media, then minced into approximately 1 mm³ small fragments using sterile scalpels. Tissue fragments were placed in a Falcon tube with warmed digestion media and then incubated for 30-45 minutes in a 37° C. shaker with agitation at 600 rpm to allow tissue to dissociate into single cells. Larger tissue fragments were allowed to settle under normal gravity for 2 minutes and the supernatant was transferred to a clean Falcon tube. An additional 3 mL of wash media with 10% FBS was added and then centrifuged. Cells were filtered through a 100 μM mesh filter as well as additional 40 μM filter to remove mouse fibroblast. Cell pellets suspended in 300 μL of ice-cold growth factor reduced Matrigel (Corning: 354230), then plated into 50 μL domes in a 24-well pre-warmed culture plate, which was allowed to solidify for 15 min at 37° C. When the Matrigel domes solidified, 500 μL of pre-warmed complete growth media was added and incubated in 5% CO₂ atmosphere at 37° C. The growth and quantity of the organoid cultures were monitored with fresh growth media added every 2 days. Organoid propagation started from adding digestion media with gentle mechanical digestion. The suspension was incubated in a Falcon tube for 30 minutes, inverted every 10 minutes, and then centrifuged at 1300 rpm for 6 minutes at 4° C. The cell pellet was resuspended in wash media with 0.1% BSA, followed by Matrigel, and then plated as domes. Wash media: advanced DMEM/F12 (Gibco: 12634010), 1M Hepes (Gibco: 15630080), 100× glutamine (Gibco: 25030149), primocin (Invivogen: ANTPM1). Digestion media: wash media/10% FBS, collagenase and dispase (Sigma: 10269638001). Growth media: wash media without FBS, 1× wnt3a/R-spondin/Noggin condition medium (L-WRN),¹³ 1× B27 supplement (Gibco: 12587-010), 10 mM nicotinamide (Sigma Aldrich: N3376), 1.25 mM N-acetylcysteine (Sigma Aldrich: A9165), 100 μg/mL primocin (Invivogen: ANTPM1), 100 ng/mL recombinant mouse Noggin (abcam: ab281818), 50 ng/mL hEGF (R&D Systems: 236EG200), 100 ng/mL human FGF (R&D Systems: 233-FB), 10 nM human gastrin I (R&D Systems: 3006/1), 500 nM A83-01 (Selleckchem: S7692), 10.5 μM Y-267632 (Selleckchem: S1049), and IgM PGE2 (R&D Systems: 2296/10).

### Immunoblot

Cells were plated in a 100 mm dish for incubation overnight before harvest for immunoblot. For drug treatment, cell confluency was checked prior to proceeding with the treatment. Cells were then harvested and lysed in a lysis buffer of 1× radioimmunoprecipitation assay (RIPA) lysis buffer (Thermo Scientific, 89900) and Halt™ Protease and Phosphatase Inhibitor Cocktail (Thermo Scientific, 78429) while maintained on ice. After centrifugation at 12,000 rpm for 15 minutes at 4° C., the protein concentration was determined using the BCA protein assay kit (Thermo Scientific, 23227). Approximately 40 μg protein samples were loaded into each well and separated by SDS-PAGE gel. After protein transfer onto nitrocellulose membranes, 5% skim milk in TBST was used to block the membranes for 1 hour at room temperature prior to incubation with primary antibodies overnight at 4° C. At the completion, a horseradish peroxidase-conjugated secondary antibody was applied at room temperature for 1 hour. The bands were visualized using Western Lightning Plus-ECL (PerkinElmer). Primary antibodies used in this study included GAPDH (Cell Signaling, 2118), pERK (Cell Signaling, 4376), ERK (Cell Signaling, 9102), cleaved PARP (Cell Signaling, 5625), and SOS1 (Cell Signaling, 5890).

To prepare siRNA-treated samples for immunoblot, cells were plated with media for 24 hours, then treated with siRNA. 12 μL lipofectamine RNAiMAX (Invitrogen: 13778150) was dilated in 200 μL Opti-MEM (Gibco: 31985070); 4 μL siRNA (10 μM) was dilated in 200 μL Opti-MEM. The mixture was incubated at room temperature for 5 minutes. After 48 hours, the cells were harvested and processed for immunoblot. siRNAs used: SOS1 (ambion: s13286, 4390824), GAPDH (ambion: s5572, 4390824), negative control (Invitrogen: AM4613).

### Organoid Drug Sensitivity Assay

Organoids were harvested with organoid cell recovery solution (Corning: 354253) and pipetted gently to dissolve Matrigel. After incubation for 15 minutes, the cells were collected and washed with 0.1% BSA/wash media. Cells were counted and resuspended in a mixture of 90% complete growth media and 10% Matrigel. 5000 cells in 30 μL media were seeded into a 96-well plate in triplicates. Of note, the plate should be prepared with 30 μL of 50% Matrigel and 50% complete growth media in each well and allowed for solidification at 37° C. for 30 minutes. Once the organoids were visible (3-7 days), drugs were added and cultured for 3 days. 40 μL of CellTiter-Glo 3D (Promega: G9681) was added and chemiluminescence was read at 360/460 nm on an Envision multi-well plate reader (PerkinElmer). After normalization of readings to DMSO-treated cells, dose-response curves were generated. IC₅₀ values were calculated using GraphPad Prism 8.4.3.

### Multiplexed Quantitative Global Proteomics

Sample Preparation: Approximately 10 millions of SW620 cells were treated with 1 μM P7 or 0.2% DMSO for 24 hours, respectively. Triplicate samples for each condition were provided as cell pellets. Cells were lysed in denaturing lysis buffer containing 8M urea, 20 mM HEPES (pH 8), 1 mM sodium orthovanadate, 2.5 mM sodium pyrophosphate and 1 mM β-glycerophosphate. A Bradford assay was carried out to determine the protein concentration. The proteins were reduced with 4.5 mM DTT and alkylated with 10 mM iodoacetamide. Trypsin digestion was carried out at room temperature overnight, and tryptic peptides were then acidified with 1% trifluoroacetic acid (TFA) and desalted with C18 Sep-Pak cartridges according to the manufacturer's procedure. TMT Labeling: 100 μg of peptide from each sample was labeled with TMTPro16plex reagent. The label incorporation was checked by LC-MS/MS and spectral counting. 98% or greater label incorporation was achieved for each channel. The samples were then pooled and lyophilized. High pH Reversed Phase Peptide Separation: After lyophilization, the peptides were re-dissolved in 250 μL of 20 mM ammonium formate (pH 10.0). The high pH reversed phase separation was performed on a Xbridge 4.6 mm×100 mm column packed with BEH C18 resin, 3.5 μm, 130 Å (Waters) The peptides were eluted as follows: 5% B (5 mM Ammonium Formate, 90% acetonitrile, pH 10.0) for 10 minutes, 5%-15% B in 5 minutes, 15-40% B in 47 minutes, 40-100% B in 5 minutes and 100% B held for 10 minutes, followed by re-equilibration at 1% B. The flow rate was 0.6 ml/min, and 24 concatenated fractions were collected. Speedvac centrifuge was used to dry the peptides. LC-MS/MS: A nanoflow ultra high performance liquid chromatography (RSLC, Dionex, Sunnyvale, CA) coupled to an electrospray bench top orbitrap mass spectrometer (Orbitrap Exploris480, Thermo, San Jose, CA) was used for tandem mass spectrometry peptide sequencing experiments. The sample was first loaded onto a pre-column (2 cm×100 μm ID packed with C18 reversed-phase resin, 5 μm, 100 Å) and washed for 8 minutes with aqueous 2% acetonitrile and 0.1% formic acid. The trapped peptides were eluted onto the analytical column, (C18, 75 μm ID×25 cm, 2 μm, 100 Å, Dionex, Sunnyvale, CA). The 120-minute gradient was programmed as: 95% solvent A (2% acetonitrile+0.1% formic acid) for 8 minutes, solvent B (90% acetonitrile+0.1% formic acid) from 5% to 38.5% in 90 minutes, then solvent B from 50% to 90% B in 7 minutes and held at 90% for 5 minutes, followed by solvent B from 90% to 5% in 1 minute and re-equilibrate for 10 minutes. The flow rate on the analytical column was 300 nl/min. nanoEasy source with FAIMS was used with spray voltage of 2100v. Data-dependent acquisition with two CV values (−45 and −65) were applied alternatively with 1.5 second cycle time each, with dynamic exclusion set at 45 seconds. The resolution settings were 120,000 and 45,000 for MS1 and MS/MS, respectively. The isolation window was 0.7 Th and HCD collision energy was set at 35%.

Data Analysis: MaxQuant (version 1.6.14.0)⁵⁴ was used to identify proteins and quantify the TMT reporter ion intensities with default settings. The protein database was downloaded in March 2021 from Uniprot.org. Samples were normalized within-plex using the Moffitt standard proteomics normalization pipeline iterative rank-order normalization (IRON).⁵⁵ Median sample was determined to be Plex_1_TMT-131, DMSO_2. All normalized values and calculations are reported as Log₂ abundances. Log₂ ratios were calculated as the difference between average treatment—average DMSO. P-values were calculated using Welch's 2-sided T-tests, assuming unequal variance. Differentially abundant proteins were determined by having a log 2 ratio ≥2 standard deviations from the mean (+0.1089/−0.1131) and a P-value <0.05. Pathway enrichment was conducted using the EnrichR R package.⁵⁶

### Immunohistochemistry of Organoids

To one or two confluent wells of organoids was added 1:5 diluted 500 μL of 5 units/mL dispase in HBSS (StemCell: 07913) and incubated at 37° C. in 5% CO₂ atmosphere. Organoids were harvested and transferred to a 1.5 mL Eppendorf tube. The pellet was collected after centrifugation at 1500 rpm for 6 min, and washed with 1×PBS. The pellet was resuspended in 1 mL of 4% PFA and incubated at 4° C. overnight. The pellet was resuspended in 100 μL of 37° C. warmed histogel, centrifuged at 1000 rpm for 30 seconds to settle the organoids. The gel was allowed to polymerize at 4° C. for 10 minutes and then the plug was transferred to a 5 mL tube containing 2 mL 70% ethanol, and sent to the histology lab. Organoids were paraffin-embedded and subjected to serial sections for hematoxylin and eosin staining and immunohistochemistry following a common procedure at the histology lab. The slides/sections were cut at 4 micron thickness using a Leica RM2245 microtome. Charged slides were used.

Immunohistochemical staining of SOS1 on organoids: Slides were stained using a Ventana Discovery Ultra automated system (Ventana Medical Systems, Tucson, AZ) as per manufacturer's protocol with proprietary reagents. Briefly, slides were deparaffinized on the automated system with Discovery Wash Solution (Ventana). Heat-induced antigen retrieval method was used in Cell Conditioning 1 Mild (Ventana). The rabbit primary antibody that reacts to SOS1 (Abcam ab140621, Cambridge, MA) was used at a 1:50 concentration in Dako antibody diluent (Carpinteria, CA) and incubated for 1.5 hours. The Ventana OmniMap anti-rabbit secondary antibody was applied for 16 minutes. The detection system used was the Ventana ChromoMap kit. Slides were counterstained with hematoxylin, then dehydrated and coverslipped as per normal laboratory protocol. Immunohistochemical staining of Annexin V: Slides were stained using a Ventana Discovery Ultra automated system (Ventana Medical Systems, Tucson, AZ) as per manufacturer's protocol with proprietary reagents. Briefly, slides were deparaffinized on the automated system with Discovery Wash Solution (Ventana). Enzyme digestion method was used in Protease 1 for 4 minutes (Ventana). The rabbit primary antibody that reacts to Annexin V (Abcam ab108321, Cambridge, MA) was used at a 1:200 concentration in Dako antibody diluent (Carpinteria, CA) and incubated for 32 minutes. The Ventana OmniMap anti-rabbit secondary antibody was applied for 16 minutes. The detection system used was the Ventana ChromoMap kit and slides were then counterstained with hematoxylin. Slides were then dehydrated and coverslipped as per normal laboratory protocol. Positive controls were those organoids treated with 5-FU and SN38.

mRNA Measurement and Quantification

Cells were treated with drugs for indicated duration of time prior to harvest for RNA extraction and purification using Qiagen RNeasy mini kit (Qiagen: 74104). The RNA samples were quantified by optical density 260/280, 260/230 readings using a spectrophotometer (NanoDrop, Thermo Fisher Scientific, Waltham, MA, USA). We used the QuantiTect reverse transcription kit (Qiagen: 205311) for cDNA synthesis and gDNA removal in accordance with the manufacturer's instructions. Quantitative RT-PCR was performed using the MX3000p Real-Time PCR System (Agilent Technologies, Santa Clara, CA, USA) to determine the mRNA expression levels of SOS1, SOS2, and GAPDH. Quantitative RT-PCR for each gene was performed using the TaqMan method with 5 μL Taqman fast advanced master mix, 3.5 μL nuclease free water, 1 μL cDNA template with 0.5 μL premade primer sets (SOS1: 4331182 Hs00893128_m1, SOS2: 4331182 Hs01127273_m1, GAPDH: 4331182 Hs03929097_g1; Thermo Scientific). This was followed by Amplitaq activation at 50° C. for 2 minutes, 95° C. for 2 minutes, and then 40 cycles at 95° C. for 1 second for denaturing and at 60° C. for 20 seconds for annealing and extension. We calculated ΔCt, defined as the difference between the crossover threshold (Ct) of the target gene and the Ct average of GAPDH for each sample.

### Abbreviations Used

ABCG1: ATP-binding cassette sub-family G member 1
CHO: Chinese hamster ovary
CRC: colorectal cancer
DCM: dichloromethane
DIEA: N,N-diisopropylethylamine
DMAP: 4-dimethylaminopyridine
DMF: dimethylformamide
DMPU: N,N′-dimethylpropyleneurea
DMSO: dimethyl sulfoxide
EDCI: 1-ethyl-3-(3-dimethylaminopropyl)carbodiimide
EGFR: epidermal growth factor receptor
GDP: guanosine diphosphate
GEF: guanine nucleotide exchange factor
GSPT1: eukaryotic peptide chain release factor GTP-binding subunit ERF3A
GTP: guanosine triphosphate
KRAS: Kirsten rat sarcoma virus
IC₅₀: half maximal inhibitory concentration
ICI: iodine monochloride
IKZF1/3: Ikaros family zinc finger protein 1/3
MAPK: mitogen-activated protein kinase
MCC: Moffitt Cancer Center
MEK: mitogen-activated protein kinase kinase
mRNA: messenger ribonucleic acid
NCT: national clinical trial
PDB: protein data bank
PDO: patient-derived organoid
pERK: phosphorylated extracellular signal-regulated kinase
PI3K: phosphoinositide 3-kinase
PROTAC: proteolysis-targeting chimera
REU: Rosetta energy units
SCAP: sterol regulatory element-binding protein cleavage-activating protein
SHP2: Src homology-2 domain-containing protein tyrosine phosphatase-2
siRNA: small interfering ribonucleic acid
SOS1: son of sevenless homologue 1
TFA: trifluoroacetic acid
VHL: von Hippel-Lindau

### REFERENCES

(1) Siegel, R. L.; Miller, K. D.; Jemal, A. Cancer Statistics, 2020. Ca Cancer J Clin 2020, 70 (1), 7-30. https://doi.org/10.3322/caac.21590.
(2) Lakatos, G.; Kohne, C.-H.; Bodoky, G. Current Therapy of Advanced Colorectal Cancer According to RAS/RAF Mutational Status. Cancer Metast Rev 2020, 1-15. https://doi.org/10.1007/s10555-020-09913-7.
(3) Uprety, D.; Adjei, A. A. KRAS: From Undruggable to a Druggable Cancer Target. Cancer Treat Rev 2020, 89, 102070. https://doi.org/10.1016/j.ctrv.2020.102070.
(4) Fakih, M. G.; Kopetz, S.; Kuboki, Y.; Kim, T. W.; Munster, P. N.; Krauss, J. C.; Falchook, G. S.; Han, S.-W.; Heinemann, V.; Muro, K.; Strickler, J. H.; Hong, D. S.; Denlinger, C. S.; Girotto, G.; Lee, M.-A.; Henary, H.; Tran, Q.; Park, J. K.; Ngarmchamnanrith, G.; Prenen, H.; Price, T. J. Sotorasib for Previously Treated Colorectal Cancers with KRAS G12C Mutation (CodeBreaK100): A Prespecified Analysis of a Single-Arm, Phase 2 Trial. Lancet Oncol 2022, 23 (1), 115-124. https://doi.org/10.1016/s1470-2045(21)00605-7.
(5) Freedman, T. S.; Sondermann, H.; Friedland, G. D.; Kortemme, T.; Bar-Sagi, D.; Marqusee, S.; Kuriyan, J. A Ras-Induced Conformational Switch in the Ras Activator Son of Sevenless. Proc National Acad Sci 2006, 103 (45), 16692-16697. https://doi.org/10.1073/pnas.0608127103.
(6) Ketcham, J. M.; Haling, J.; Khare, S.; Bowcut, V.; Briere, D. M.; Burns, A. C.; Gunn, R. J.; Ivetac, A.; Kuehler, J.; Kulyk, S.; Laguer, J.; Lawson, J. D.; Moya, K.; Nguyen, N.; Rahbaek, L.; Saechao, B.; Smith, C. R.; Sudhakar, N.; Thomas, N. C.; Vegar, L.; Vanderpool, D.; Wang, X.; Yan, L.; Olson, P.; Christensen, J. G.; Marx, M. A. Design and Discovery of MRTX0902, a Potent, Selective, Brain-Penetrant, and Orally Bioavailable Inhibitor of the SOS1:KRAS Protein-Protein Interaction. J Med Chem 2022. https://doi.org/10.1021/acs.jmedchem.2c00741.
(7) Moore, A. R.; Rosenberg, S. C.; McCormick, F.; Malek, S. RAS-Targeted Therapies: Is the Undruggable Drugged? Nat Rev Drug Discov 2020, 19 (8), 533-552. https://doi.org/10.1038/s41573-020-0068-6.
(8) Depeille, P.; Henricks, L. M.; Ven, R. A. H. van de; Lemmens, E.; Wang, C.-Y.; Math, M.; Werb, Z.; Haigis, K. M.; Donner, D.; Warren, R.; Roose, J. P. RasGRP1 Opposes Proliferative EGFR-SOS1-Ras Signals and Restricts Intestinal Epithelial Cell Growth. Nat Cell Biol 2015, 17 (6), 804-815. https://doi.org/10.1038/ncb3175.
(9) Jeng, H.-H.; Taylor, L. J.; Bar-Sagi, D. Sos-Mediated Cross-Activation of Wild-Type Ras by Oncogenic Ras Is Essential for Tumorigenesis. Nat Commun 2012, 3 (1), 1168. https://doi.org/10.1038/ncomms2173.
(10) You, X.; Kong, G.; Ranheim, E. A.; Yang, D.; Zhou, Y.; Zhang, J. Unique Dependence on Sos1 in KrasG12D-Induced Leukemogenesis. Blood 2018, 132 (24), 2575-2579. https//doi.org/10.1182/blood-2018-09-874107.
(11) Abbott, J. R.; Hodges, T. R.; Daniels, R. N.; Patel, P. A.; Kennedy, J. P.; Howes, J. E.; Akan, D. T.; Burns, M. C.; Sai, J.; Sobolik, T.; Beesetty, Y.; Lee, T.; Rossanese, O. W.; Phan, J.; Waterson, A. G.; Fesik, S. W. Discovery of Aminopiperidine Indoles That Activate the Guanine Nucleotide Exchange Factor SOS1 and Modulate RAS Signaling. J Med Chem 2018, 61 (14), 6002-6017. https://doi.org/10.1021/acs.jmedchem.8b00360.
(12) Abbott, J. R.; Patel, P. A.; Howes, J. E.; Akan, D. T.; Kennedy, J. P.; Burns, M. C.; Browning, C. F.; Sun, Q.; Rossanese, O. W.; Phan, J.; Waterson, A. G.; Fesik, S. W. Discovery of Quinazolines That Activate SOS1-Mediated Nucleotide Exchange on RAS. Acs Med Chem Lett 2018, 9 (9), 941-946. https://doi.org/10.1021/acsmedchemlett.8b00296.
(13) Akan, D. T.; Howes, J. E.; Sai, J.; Arnold, A. L.; Beesetty, Y.; Phan, J.; Olejniczak, E. T.; Waterson, A. G.; Fesik, S. W. Small Molecule SOS1 Agonists Modulate MAPK and PI3K Signaling via Independent Cellular Responses. Acs Chem Biol 2019, 14 (3), 325-331. https://doi.org/10.1021/acschembio.8b00869.
(14) Burns, M. C.; Sun, Q.; Daniels, R. N.; Camper, D.; Kennedy, J. P.; Phan, J.; Olejniczak, E. T.; Lee, T.; Waterson, A. G.; Rossanese, O. W.; Fesik, S. W. Approach for Targeting Ras with Small Molecules That Activate SOS-Mediated Nucleotide Exchange. Proc National Acad Sci 2014, 1)1 (9), 3401-3406. https://doi.org/10.1073/pnas.1315798111.
(15) Hillig, R. C.; Sautier, B.; Schroeder, J.; Moosmayer, D.; Hilpmann, A.; Stegmann, C. M.; Werbeck, N. D.; Briem, H.; Boemer, U.; Weiske, J.; Badock, V.; Mastouri, J.; Petersen, K.; Siemeister, G.; Kahmann, J. D.; Wegener, D.; Böhnke, N.; Eis, K.; Graham, K.; Wortmann, L.; Nussbaum, F. von; Bader, B. Discovery of Potent SOS1 Inhibitors That Block RAS Activation via Disruption of the RAS-SOS1 Interaction. Proc National Acad Sci 2019, 116 (7), 2551-2560. https://doi.org/10.1073/pnas.1812963116.
(16) Hodges, T. R.; Abbott, J. R.; Little, A. J.; Sarkar, D.; Salovich, J. M.; Howes, J. E.; Akan, D. T.; Sai, J.; Arnold, A. L.; Browning, C.; Burns, M. C.; Sobolik, T.; Sun, Q.; Beesetty, Y.; Coker, J. A.; Scharn, D.; Stadtmueller, H.; Rossanese, O. W.; Phan, J.; Waterson, A. G.; McConnell, D. B.; Fesik, S. W. Discovery and Structure-Based Optimization of Benzimidazole-Derived Activators of SOS1-Mediated Nucleotide Exchange on RAS. J Med Chem 2018, 61 (19), 8875-8894. https://doi.org/10.1021/acs.jmedchem.8b01108.
(17) Hofmann, M. H.; Gmachl, M.; Ramharter, J.; Savarese, F.; Gerlach, D.; Marszalek, J.
R.; Sanderson, M. P.; Kessler, D.; Trapani, F.; Arnhof, H.; Rumpel, K.; Botesteanu, D.-A.; Ettmayer, P.; Gerstberger, T.; Kofink, C.; Wunberg, T.; Zoephel, A.; Fu, S.-C.; Teh, J. L.; Böttcher, J.; Pototschnig, N.; Schachinger, F.; Schipany, K.; Lieb, S.; Vellano, C. P.; O'Connell, J. C.; Mendes, R. L.; Moll, J.; Petronczki, M.; Heffernan, T. P.; Pearson, M.; McConnell, D. B.; Kraut, N. BI-3406, a Potent and Selective SOS1-KRAS Interaction Inhibitor, Is Effective in KRAS-Driven Cancers through Combined MEK Inhibition. Cancer Discov 2021, 11 (1), 142-157. https://doi.org/10.1158/2159-8290.cd-20-0142.
(18) Sarkar, D.; Olejniczak, E. T.; Phan, J.; Coker, J. A.; Sai, J.; Arnold, A.; Beesetty, Y.; Waterson, A. G.; Fesik, S. W. Discovery of Sulfonamide-Derived Agonists of SOS1-Mediated Nucleotide Exchange on RAS Using Fragment-Based Methods. J Med Chem 2020, 63 (15), 8325-8337. https://doi.org/10.1021/acs.jmedchem.0c00511.
(19) Ramharter, J.; Kessler, D.; Ettmayer, P.; Hofmann, M. H.; Gerstberger, T.; Gmachl, M.; Wunberg, T.; Kofink, C.; Sanderson, M.; Arnhof, H.; Bader, G.; Rumpel, K.; Zöphel, A.; Schnitzer, R.; Böttcher, J.; O'Connell, J. C.; Mendes, R. L.; Richard, D.; Pototschnig, N.; Weiner, I.; Hela, W.; Hauer, K.; Haering, D.; Lamarre, L.; Wolkerstorfer, B.; Salamon, C.; Werni, P.; Munico-Martinez, S.; Meyer, R.; Kennedy, M. D.; Kraut, N.; McConnell, D. B. One Atom Makes All the Difference: Getting a Foot in the Door between SOS1 and KRAS. J Med Chem 2021. https://doi.org/10.1021/acs.jmedchem.0c01949.
(20) Cromm, P. M.; Crews, C. M. Targeted Protein Degradation: From Chemical Biology to Drug Discovery. Cell Chem Biol 2017, 24 (9), 1181-1190. https://doi.org/10.1016/j.chembiol.2017.05.024.
(21) Kostic, M.; Jones, L. H. Critical Assessment of Targeted Protein Degradation as a Research Tool and Pharmacological Modality. Trends Pharmacol Sci 2020, 41 (5), 305-317. https://doi.org/10.1016/j.tips.2020.02.006.
(22) Yang, X.; Yin, H.; Kim, R. D.; Fleming, J. B.; Xie, H. Preclinical and Clinical Advances of Targeted Protein Degradation as a Novel Cancer Therapeutic Strategy: An Oncologist Perspective. Target Oncol 2021, 16 (1), 1-12. https://doi.org/10.1007/s11523-020-00782-2.
(23) Wang, M.; Lu, J.; Wang, M.; Yang, C.-Y.; Wang, S. Discovery of SHP2-D26 as a First, Potent, and Effective PROTAC Degrader of SHP2 Protein. J Med Chem 2020, 63 (14), 7510-7528. https://doi.org/10.1021/acs.jmedchem.0c00471.
(24) clinicaltrials.gov. A Study to Test Different Doses of BI 1701963 Alone and Combined With Trametinib in Patients With Different Types of Advanced Cancer (Solid Tumours With KRAS Mutation). https://clinicaltrials.gov/ct2/show/NCT04111458 (accessed 2022-07-06).
(25) Singh, J.; Deng, Z.; Narale, G.; Chuaqui, C. Structural Interaction Fingerprints: A New Approach to Organizing, Mining, Analyzing, and Designing Protein-Small Molecule Complexes. Chem Biol Drug Des 2006, 67 (1), 5-12. https://doi.org/10.1111/j.1747-0285.2005.00323.x.
(26) Schrödinger Release 2020 March: Maestro, Schrödinger, LLC, New York, NY, 2020.
(27) CREGG, J. J.; BUCKL, A.; Aay, N.; TAMBO-ONG, A. A.; Koltun, E. S.; Gill, A. L.; THOMPSON, S.; GLIEDT, M. J. WO2020180770A1: Bicyclic heterocyclyl compounds and uses thereof. https://patents.google.com/patent/WO2020180770A1/en (accessed 2022 Jul. 1).
(28) Zaidman, D.; Prilusky, J.; London, N. PRosettaC: Rosetta Based Modeling of PROTAC Mediated Ternary Complexes. J Chem Inf Model 2020, 60 (10), 4894-4903. https://doi.org/10.1021/acs.jcim.0c00589.
(29) Gray, J. J.; Moughon, S.; Wang, C.; Schueler-Furman, O.; Kuhlman, B.; Rohl, C. A.; Baker, D. Protein-Protein Docking with Simultaneous Optimization of Rigid-Body Displacement and Side-Chain Conformations. J Mol Biol 2003, 331 (1), 281-299. https://doi.org/10.1016/s0022-2836(03)00670-3.
(30) Donovan, K. A.; Ferguson, F. M.; Bushman, J. W.; Eleuteri, N. A.; Bhunia, D.; Ryu, S.; Tan, L.; Shi, K.; Yue, H.; Liu, X.; Dobrovolsky, D.; Jiang, B.; Wang, J.; Hao, M.; You, I.; Teng, M.; Liang, Y.; Hatcher, J.; Li, Z.; Manz, T. D.; Groendyke, B.; Hu, W.; Nam, Y.; Sengupta, S.; Cho, H.; Shin, I.; Agius, M. P.; Ghobrial, I. M.; Ma, M. W.; Che, J.; Buhrlage, S. J.; Sim, T.; Gray, N. S.; Fischer, E. S. Mapping the Degradable Kinome Provides a Resource for Expedited Degrader Development. Cell 2020. https://doi.org/10.1016/j.cell.2020.10.038.
(31) Zhou, C.; Fan, Z.; Zhou, Z.; Li, Y.; Cui, R.; Liu, C.; Zhou, G.; Diao, X.; Jiang, H.; Zheng, M.; Zhang, S.; Xu, T. Discovery of the First-in-Class Agonist-Based SOS1 PROTACs Effective in Human Cancer Cells Harboring Various KRAS Mutations. J Med Chem 2021. https://doi.org/10.1021/acs.jmedchem.Ic01774.
(32) Ishoey, M.; Chorn, S.; Singh, N.; Jaeger, M. G.; Brand, M.; Paulk, J.; Bauer, S.; Erb, M. A.; Parapatics, K.; Müller, A. C.; Bennett, K. L.; Ecker, G. F.; Bradner, J. E.; Winter, G. E. Translation Termination Factor GSPT1 Is a Phenotypically Relevant Off-Target of Heterobifunctional Phthalimide Degraders. Acs Chem Biol 2018, 13 (3), 553-560. https://doi.org/10.1021/acschembio.7b00969.
(33) Matyskiela, M. E.; Lu, G.; Ito, T.; Pagarigan, B.; Lu, C.-C.; Miller, K.; Fang, W.; Wang, N.-Y.; Nguyen, D.; Houston, J.; Carmel, G.; Tran, T.; Riley, M.; Nosaka, L.; Lander, G. C.; Gaidarova, S.; Xu, S.; Ruchelman, A. L.; Handa, H.; Carmichael, J.; Daniel, T. O.; Cathers, B. E.; Lopez-Girona, A.; Chamberlain, P. P. A Novel Cereblon Modulator Recruits GSPT1 to the CRL4CRBN Ubiquitin Ligase. Nature 2016, 535 (7611), 252-257. https://doi.org/10.1038/nature18611.
(34) Gabitova, L.; Restifo, D.; Gorin, A.; Manocha, K.; Handorf, E.; Yang, D.-H.; Cai, K. Q.; Klein-Szanto, A. J.; Cunningham, D.; Kratz, L. E.; Herman, G. E.; Golemis, E. A.; Astsaturov, I. Endogenous Sterol Metabolites Regulate Growth of EGFR/KRAS-Dependent Tumors via LXR. Cell Reports 2015, 12 (11), 1927-1938. https://doi.org/10.1016/j.celrep.2015.08.023.
(35) Lee, S. H.; Lee, J.-H.; Im, S.-S. The Cellular Function of SCAP in Metabolic Signaling. Exp Mol Medicine 2020, 52 (5), 724-729. https://doi.org/10.1038/s12276-020-0430-0.
(36) Mulay, V.; Wood, P.; Manetsch, M.; Darabi, M.; Cairns, R.; Hoque, M.; Chan, K. C.; Reverter, M.; Álvarez-Guaita, A.; Rye, K.-A.; Rentero, C.; Heeren, J.; Enrich, C.; Grewal, T. Inhibition of Mitogen-Activated Protein Kinase Erk1/2 Promotes Protein Degradation of ATP Binding Cassette Transporters A1 and G1 in CHO and HuH7 Cells. Plos One 2013, 8 (4), e62667. https://doi.org/10.1371/joumal.pone.0062667.
(37) Sheffels, E.; Kortum, R. L. Breaking Oncogene Addiction: Getting RTK/RAS-Mutated Cancers off the SOS. J Med Chem 2021, 64 (10), 6566-6568. https://doi.org/10.1021/acs.jmedchem.Ic00698.
(38) Kawaguchi, M.; Okabe, T.; Okudaira, S.; Nishimasu, H.; Ishitani, R.; Kojima, H.; Nureki, O.; Aoki, J.; Nagano, T. Screening and X-ray Crystal Structure-Based Optimization of Autotaxin (ENPP2) Inhibitors, Using a Newly Developed Fluorescence Probe. Acs Chem Biol 2013, 8 (8), 1713-1721. https://doi.org/10.1021/cb400150c.
(39) Liu, J.; Yuan, L.; Ruan, Y.; Deng, B.; Yang, Z.; Ren, Y.; Li, L.; Liu, T.; Zhao, H.; Mai, R.; Chen, J. Novel CRBN-Recruiting Proteolysis-Targeting Chimeras as Degraders of Stimulator of Interferon Genes with In Vivo Anti-Inflammatory Efficacy. J Med Chem 2022, 65 (9), 6593-6611. https://doi.org/10.1021/acs.jmedchem.Ic01948.
(40) Devine, W. G.; Diaz-Gonzalez, R.; Ceballos-Perez, G.; Rojas, D.; Satoh, T.; Tear, W.; Ranade, R. M.; Barros-Álvarez, X.; Hol, W. G. J.; Buckner, F. S.; Navarro, M.; Pollastri, M. P. From Cells to Mice to Target: Characterization of NEU-1053 (SB-443342) and Its Analogues for Treatment of Human African Trypanosomiasis. Acs Infect Dis 2017, 3 (3), 225-236. https://doi.org/10.1021/acsinfecdis.6b00202.
(41) Clercq, D. J. H. D.; Risseeuw, M. D. P.; Karalic, I.; Smet, A. D.; Defever, D.; Tavernier, J.; Lievens, S.; Calenbergh, S. V. Alternative Reagents for Methotrexate as Immobilizing Anchor Moieties in the Optimization of MASPIT: Synthesis and Biological Evaluation. Chembiochem 2015, 16 (5), 834-843. https://doi.org/10.1002/cbic.201402702.
(42) Chen, H.; Chen, F.; Pei, S.; Gou, S. Pomalidomide Hybrids Act as Proteolysis Targeting Chimeras: Synthesis, Anticancer Activity and B-Raf Degradation. Bioorg Chem 2019, 87, 191-199. https://doi.org/10.1016/j.bioorg.2019.03.035.
(43) Lu, Y.; Sun, D.; Xiao, D.; Shao, Y.; Su, M.; Zhou, Y.; Li, J.; Zhu, S.; Lu, W. Design, Synthesis, and Biological Evaluation of HDAC Degraders with CRBN E3 Ligase Ligands. Molecules 2021, 26 (23), 7241. https://doi.org/10.3390/molecules26237241.
(44) Liu, J.; Plewe, M.; Wang, J.; Han, X.; Chen, L. Cyclic-AMP Response Element Binding Protein (CBP) and/or Adenoviral E1A Binding Protein of 300 KDA (P300) Degradation Compounds and Methods of Use, Mar. 9, 2020.
(45) Robbins, D.; Peng, G.; Mihalic, J.; Sands, A. Bifunctional Compounds for Degrading BTK via Ubiquitin Proteasome Pathway, May 14, 2021.
(46) Chamberlain, P. P.; Lopez-Girona, A.; Miller, K.; Carmel, G.; Pagarigan, B.; Chie-Leon, B.; Rychak, E.; Corral, L. G.; Ren, Y. J.; Wang, M.; Riley, M.; Delker, S. L.; Ito, T.; Ando, H.; Mori, T.; Hirano, Y.; Handa, H.; Hakoshima, T.; Daniel, T. O.; Cathers, B. E.
Structure of the Human Cereblon-DDB1-Lenalidomide Complex Reveals Basis for Responsiveness to Thalidomide Analogs. Nat Struct Mol Biol 2014, 21 (9), 803-809. https://doi.org/10.1038/nsmb.2874.
(47) Schrödinger Release 2020 March: Protein Preparation Wizard; Epik, Schrödinger, LLC, New York, NY, 2020; Impact, Schrödinger, LLC, New York, NY; Prime, Schrödinger, LLC, New York, NY, 2020.
(48) Pettersen, E. F.; Goddard, T. D.; Huang, C. C.; Meng, E. C.; Couch, G. S.; Croll, T. I.; Morris, J. H.; Ferrin, T. E. UCSF ChimeraX: Structure Visualization for Researchers, Educators, and Developers. Protein Sci 2021, 30 (1), 70-82. https://doi.org/10.1002/pro.3943.
(49) R Core Team (2021). R: A Language and Environment for Statistical Computing. (Version 4.1) [Computer Software]. Retrieved from Https://Cran.r-Project.Org. (R Packages Retrieved from MRAN Snapshot 2022-01-01).
(50) The Jamovi Project (2022). Jamovi. (Version 2.3) [Computer Software]. Retrieved from Https://Www.Jamovi.Org.
(51) van de Wetering, M.; Francies, H. E.; Francis, J. M.; Bounova, G.; Iorio, F.; Pronk, A.; van Houdt, W.; van Gorp, J.; Taylor-Weiner, A.; Kester, L.; McLaren-Douglas, A.; Blokker, J.; Jaksani, S.; Bartfeld, S.; Volckman, R.; van Sluis, P.; Li, V. S. W.; Seepo, S.; Sekhar Pedamallu, C.; Cibulskis, K.; Carter, S. L.; McKenna, A.; Lawrence, M. S.; Lichtenstein, L.; Stewart, C.; Koster, J.; Versteeg, R.; van Oudenaarden, A.; Saez-Rodriguez, J.; Vries, R. G. J.; Getz, G.; Wessels, L.; Stratton, M. R.; McDermott, U.; Meyerson, M.; Garnett, M. J.; Clevers, H. Prospective Derivation of a Living Organoid Biobank of Colorectal Cancer Patients. Cell 2015, 161 (4), 933-945. https://doi.org/10.1016/j.cell.2015.03.053.
(52) Vlachogiannis, G.; Hedayat, S.; Vatsiou, A.; Jamin, Y.; Fernández-Mateos, J.; Khan, K.; Lampis, A.; Eason, K.; Huntingford, I.; Burke, R.; Rata, M.; Koh, D.-M.; Tunariu, N.; Collins, D.; Hulkki-Wilson, S.; Ragulan, C.; Spiteri, I.; Moorcraft, S. Y.; Chau, I.; Rao, S.; Watkins, D.; Fotiadis, N.; Bali, M.; Darvish-Damavandi, M.; Lote, H.; Eltahir, Z.; Smyth, E. C.; Begum, R.; Clarke, P. A.; Hahne, J. C.; Dowsett, M.; Bono, J. de; Workman, P.; Sadanandam, A.; Fassan, M.; Sansom, O. J.; Eccles, S.; Starling, N.; Braconi, C.; Sottoriva, A.; Robinson, S. P.; Cunningham, D.; Valeri, N. Patient-Derived Organoids Model Treatment Response of Metastatic Gastrointestinal Cancers. Science 2018, 359 (6378), 920-926. https://doi.org/10.1126/science.aao2774.
(53) Miyoshi, H.; Stappenbeck, T. S. In Vitro Expansion and Genetic Modification of Gastrointestinal Stem Cells in Spheroid Culture. Nat Protoc 2013, 8 (12), 2471-2482. https://doi.org/10.1038/nprot.2013.153.
(54) Cox, J.; Mann, M. MaxQuant Enables High Peptide Identification Rates, Individualized p.p.b.-Range Mass Accuracies and Proteome-Wide Protein Quantification. Nat Biotechnol 2008, 26 (12), 1367-1372. https://doi.org/10.1038/nbt.1511.
(55) Welsh, E. A.; Eschrich, S. A.; Berglund, A. E.; Fenstermacher, D. A. Iterative Rank-Order Normalization of Gene Expression Microarray Data. Bmc Bioinformatics 2013, 14 (1), 153. https://doi.org/10.1186/1471-2105-14-153.
(56) Xie, Z.; Bailey, A.; Kuleshov, M. V.; Clarke, D. J. B.; Evangelista, J. E.; Jenkins, S. L.; Lachmann, A.; Wojciechowicz, M. L.; Kropiwnicki, E.; Jagodnik, K. M.; Jeon, M.; Ma'ayan, A. Gene Set Knowledge Discovery with Enrichr. Curr Protoc 2021, 1 (3), e90. https://doi.org/10.1002/cpz1.90.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “Including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A compound of Formula I or Formula II 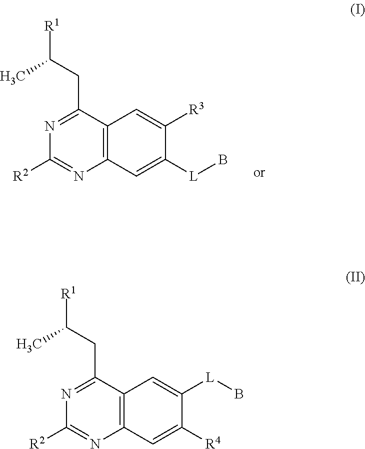
or a pharmaceutically acceptable salt or derivative thereof,
wherein:
R¹ is aryl optionally substituted with one or more groups independently selected from halo, amino, C₁-C₆ alkyl, and C₁-C₆ haloalkyl;
R² is selected from H or C₁-C₆ alkyl;
R³ and R⁴ are each C₁-C₆ alkoxy;
L is a linker; and
B is an E3 ubiquitin ligase-recruiting moiety.

2. The compound of claim 1, wherein R¹ is unsubstituted phenyl.

3. The compound of claim 1, wherein R¹ is phenyl substituted with C₁-C₆ haloalkyl.

4. The compound of claim 3, wherein R¹ is phenyl substituted with trifluoromethyl.

5. The compound of claim 1, wherein R¹ is phenyl substituted with amino and C₁-C₆ haloalkyl.

6. The compound of claim 5, wherein R¹ is phenyl substituted with amino and trifluoromethyl.

7. The compound of claim 1, wherein R¹ is selected from:

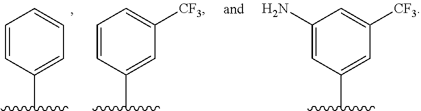

8. The compound of claim 1, wherein R² is H.

9. The compound of claim 1, wherein R² is C₁-C₆ alkyl.

10. The compound of claim 9, wherein R² is methyl.

11. The compound of claim 1, R³ or R⁴ is methoxy.

12. (canceled)

13. The compound of claim 1, wherein the linker is selected from the group consisting of a moiety of Formula L1, Formula L2, Formula L3, Formula L4, Formula L5, Formula L6, Formula L7, Formula L8, Formula L9, or Formula L10: 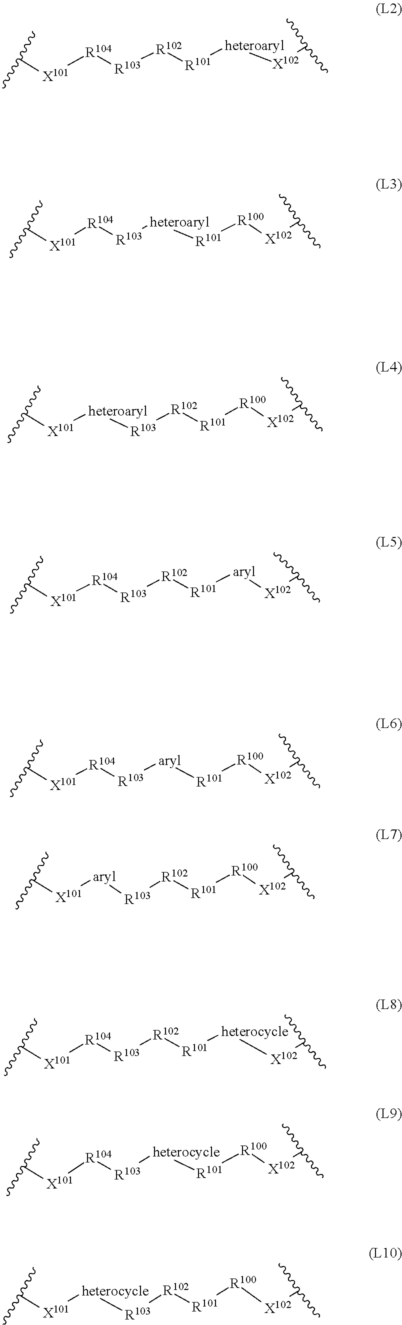
wherein:
X¹⁰¹ and X¹⁰² are independently at each occurrence selected from a bond, aryl, heteroaryl, cycloalkyl, heterocycle, NR¹³⁰, C(R¹³⁰)₂, O, C(O), and S;
R¹⁰⁰, R¹⁰¹, R¹⁰², R¹⁰³, and R¹⁰⁴ are independently at each occurrence selected from the group consisting of a bond, alkyl, —C(O)—, —C(O)O—, —OC(O)—, —SO₂—, —S(O)—, C(S)—, —C(O)NR¹³⁰—, —NR¹³⁰C(O)—, —O—, —S—, —NR¹³⁰—, —C(R¹³⁰R¹³⁰)—, —P(O)(OR¹⁰⁶))—, —R(O)(OR¹⁰⁶)—, alkenyl, alkynyl, haloalkyl, alkoxy, aryl, heterocycloalkyl, cycloalkyl, heteroaryl, lactic acid, or glycolic acid, each of which may be optionally substituted with one or more (for example, 1, 2, 3, or 4) substituents independently selected from R¹⁴⁰;
R¹⁰⁶ is independently at each occurrence selected from the group consisting of hydrogen, alkyl, arylalkyl, heteroarylalkyl, alkenyl, alkynyl, aryl, heteroaryl, cycloalkyl, or heterocycloalkyl;
R¹³⁰ is independently as each occurrence selected from the group consisting of hydrogen, alkyl, cycloalkyl, heterocycloalkyl, aryl, heteroaryl, —C(O)H, —C(O)OH, —C(O)alkyl, —C(O)Oalkyl, —C(O)(cycloalkyl, heterocycloalkyl, aryl, or heteroaryl), —C(O)O(cycloalkyl, heterocycloalkyl, aryl, or heteroaryl), alkenyl, or alkynyl; and
R¹⁴⁰ is independently at each occurrence selected from the group consisting of hydrogen, alkyl, alkenyl, alkynyl, fluoro, bromo, chloro, hydroxyl, alkoxy, azide, amino cyano, —NH(alkyl, cycloalkyl, heterocyloalkyl, aryl, or heteroaryl), —N(independently alkyl, cycloalkyl, heterocycloalkyl, aryl, or heteroaryl), —NHSO₂(alkyl, cycloalkyl, heterocycloalkyl, aryl, or heteroaryl), —N(alkyl, cycloalkyl, heterocycloalkyl, aryl, or heteroaryl)SO₂alkyl, —NHSO₂alkenyl, —N(alkyl)SO₂alkenyl, —NHSO₂alkynyl, —N(alkyl)SO₂alkynyl, haloalkyl, cycloalkyl, heterocycloalkyl, aryl, or heteroaryl.

14. The compound of claim 1, wherein B is selected from 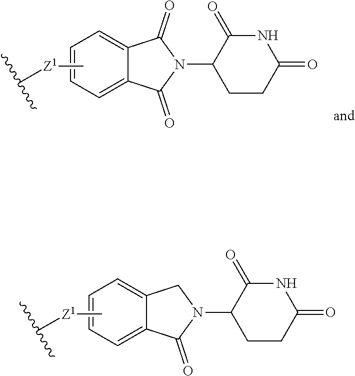
wherein Z⁵ is selected from O, N(R^{a}), and CH₂.

15. The compound of claim 1, wherein B is selected from

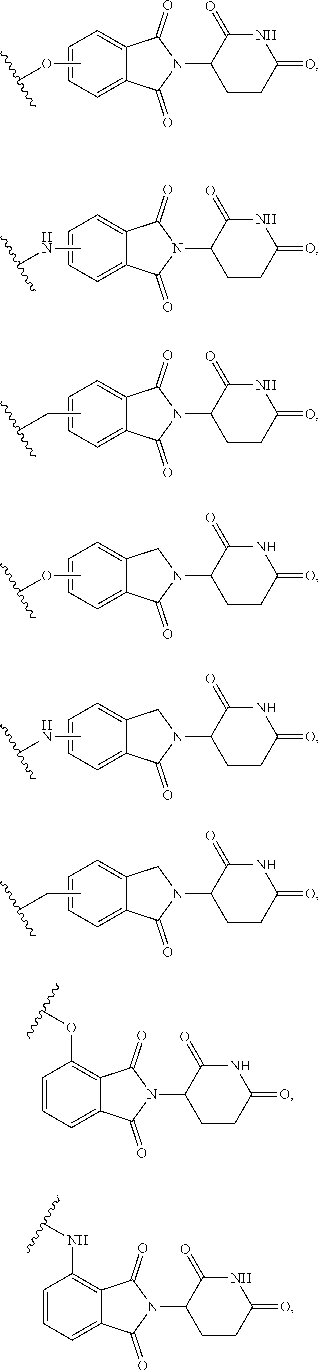

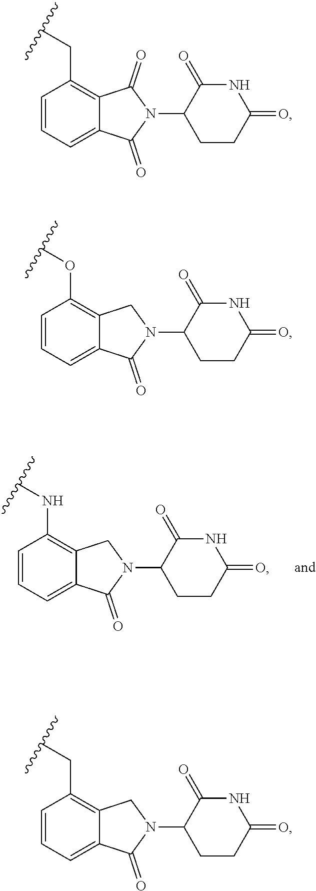 [SMILES: *Cc1cccc2c1C(=O)N(C1CCC(=O)NC1=O)C2=O ; *Oc1cccc2c1CN(C1CCC(=O)NC1=O)C2=O ; *Nc1cccc2c1CN(C1CCC(=O)NC1=O)C2=O ; *Cc1cccc2c1CN(C1CCC(=O)NC1=O)C2=O]

16. (canceled)

17. The compound of claim 1, wherein B is selected from

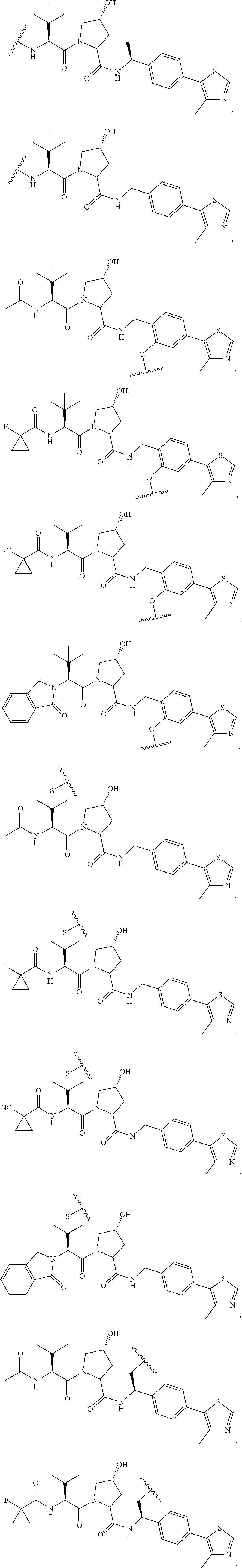

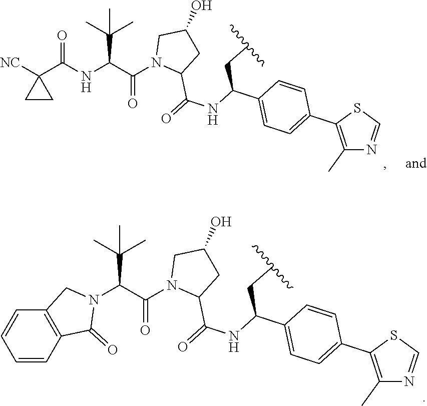 [SMILES: *C[C@H](NC(=O)C1C[C@@H](O)CN1C(=O)[C@@H](NC(=O)C1(C#N)CC1)C(C)(C)C)c1ccc(-c2scnc2C)cc1 ; *C[C@H](NC(=O)C1C[C@@H](O)CN1C(=O)[C@@H](N1Cc2ccccc2C1=O)C(C)(C)C)c1ccc(-c2scnc2C)cc1]

18. A pharmaceutical composition comprising a compound of claim 1, or a pharmaceutically acceptable salt or derivative thereof, and a pharmaceutically acceptable carrier or excipient.

19. A method of treating cancer in a subject in need thereof comprising administering to the subject a therapeutically effective amount of a compound of claim 1, or a pharmaceutically acceptable salt or derivative thereof.

20. The method of claim 19, wherein the cancer is a KRAS-mutant cancer.

21. The method of claim 19, wherein the cancer is KRAS-mutant colorectal cancer.

22. The method of claim 19, wherein the subject is a human.
